(19) United States
(12) Patent Application Publication
(10) Pub. No.: US 2022/0361758 A1
(43) Pub. Date: Nov. 17, 2022

(54) DRUG ADMINISTRATION DEVICES THAT COMMUNICATE WITH EXTERNAL SYSTEMS AND/OR OTHER DEVICES

(71) Applicant: JANSSEN PHARMACEUTICALS, INC., Titusville, NJ (US)

(72) Inventors: Francesco N. ALBERTINI, Pleasanton, CA (US); Gregory J. BAKOS, Mason, OH (US); Jeffrey DANIEL, Cincinnati, OH (US); Anthony R. DIUBALDI, Jackson, NJ (US); James A. FLEMING, Bethlehem, PA (US); Jason L. HARRIS, Lebanon, OH (US); Kevin L. HOUSER, Navarre, FL (US); Emma Louise HUBERT, San Jose, CA (US); Michael HUTCHINSON, King of Prussia, PA (US); David KALIKHMAN, Huntingdon Valley, PA (US); Monica A. KAPIL, San Jose, CA (US); Wouter Jacques Noel LEQUIEU, Lievegem (BE); David RAMOS, Orange Park, FL (US); Ian SCRIMGEOUR, East Lothian (GB); Frederick E. SHELTON, IV, Cincinnati, OH (US); Jaskaran SINGH, San Diego, CA (US); Steven M. VESOLE, San Bruno, CA (US); Jingli WANG, San Jose, CA (US); Hong YAN, Livermore, CA (US)

(21) Appl. No.: 17/763,033

(22) PCT Filed: Sep. 24, 2020
(86) PCT No.: PCT/IB2020/058959

§ 371 Date: Mar. 23, 2022

Related U.S. Application Data
(60) Provisional application No. 62/905,465, filed on Sep. 25, 2019.
(60) Provisional application No. 62/905,468, filed on Sep. 25, 2019.
(60) Provisional application No. 62/905,471, filed on Sep. 25, 2019.
(60) Provisional application No. 62/905,473, filed on Sep. 25, 2019.
(60) Provisional application No. 63/020,940, filed on May 6, 2020.

(51) Int. Cl. A61B 5/024; A61B 5/00

(52) U.S. Cl. CPC A61B 5/024; A61B 5/0022; A61B 5/1118

(57) ABSTRACT

In general, drug administration devices configured to communicate with networks and external devices are provided. In an exemplary embodiment, a drug administration device is configured to adjust an operational parameter of the drug administration device based on a data packet formed from drug administration data ancillary data received from networks and/or external devices. In another example embodiment, a drug administration device is configured to scan for and establish communications with at least one external device. In another example embodiment, a method includes assessing whether to update a control program on a drug administration device from a network or external device. In another example embodiment, a system includes a first drug administration device configured to communicate with a second drug administration device to optimize a drug treatment.

### FIELD

The embodiments described herein relate to a device for administering and/or provision of a drug. The present disclosure further relates to a system in which the device can be used, and a method of administration, and a further method associated with the system.

### BACKGROUND

Pharmaceutical products (including large and small molecule pharmaceuticals, hereinafter “drugs”) are administered to patients in a variety of different ways for the treatment of specific medical indications. Regardless of the manner of the administration, care must be taken when administering drugs to avoid adverse effects on the patient. For example, care must be taken not to administer more than a safe amount of the drug to the patient. This requires consideration of the amount of dose given and the time frame over which the dose is delivered, sometimes in relation to previous doses, or doses of other drugs. Moreover, care must be taken not to inadvertently administer an incorrect drug to the patient, or drugs that have degraded due to their age or storage conditions. All of these considerations can be conveyed in guidance associated with the specific drugs or drug combinations. However, this guidance is not always followed correctly, for example due to mistakes, such as human error. This can lead to adverse effects on the patient or result in inappropriate drug administration, for example insufficient or excessive volume of drug being administered for the specific medical indication.

Patients rarely share the same medical characteristics. For example, patients generally have different ages, weights, general states of health, and medical histories. Therefore the same illness tends to affect patients differently. Thus, while guidance supplied with specific drugs may aid a medical practitioner or patient in determining a suitable dosage amount, dosage frequency, and dosage time (dosage regimen) it will not necessarily inform the medical practitioner or patient of the optimum dosage for a particular patient. In order to determine the optimum dosage, the medical practitioner or patient would have to measure some or all possible factors affecting a patient and consider how the different factors interact. This is often impossible, and so medical practitioners or patients have to make a best guess as to the optimum dosage based on information that they have observed about the patient. These best guesses will rarely result in timely administration of an optimum dosage. Moreover, because the best guess is based on data observed by the medical practitioner or patient, there is an undesirable element of subjectivity and possibility of user error when determining or attempting to administer the best guess dosage.

In relation to how a drug is administered to the patient, there are various dosage forms that can be used. For example, these dosage forms may include parenteral, inhalational, oral, ophthalmic, topical, nasal, and suppository forms of one or more drugs.

The dosage forms can be administered directly to the patient via a drug administration device. There are a number of different types of drug administration devices commonly available for delivery of the various dosage forms including: syringes, injection devices (e.g., autoinjectors, jet injectors, and infusion pumps), nasal spray devices, and inhalers.

### SUMMARY

In a first aspect, a computer-implemented method of generating a data packet comprising drug administration data and ancillary data is provided. In one embodiment, the drug administration data has a first data format and relates to parameters of a drug administration device. The method includes establishing communication between the drug administration device and at least one of a network and an external device, receiving the ancillary data from the at least one of the network and the external device at the drug administration device, and generating the data packet at the drug administration device. The data packet is generated at the drug administration device by filtering the ancillary data such that ancillary data relating to a user parameter remains, converting at least the ancillary data relating to the user parameter into the first data format, and combining the drug administration data and the ancillary data relating to the user parameter.

The method can vary in any number of ways. For example, the method can include the drug administration device adjusting at least one operational parameter of the drug administration device based on the data packet. In at least some embodiments, adjusting the at least one operational parameter can include comparing a data packet value to a reference data packet value, and adjusting the at least one operational parameter of the drug administration device based on the comparison. In at least some embodiments, adjusting the at least one operational parameter can include comparing a plurality of data packet values to a plurality of reference data packet values, and adjusting the at least one operational parameter of the drug administration device based on the comparisons. The reference data packet value can relate to at least one of a physiological condition of a user, a psychological condition of a user, and an environmental conditional of a user. The at least one operational parameter can relate to at least one of a dosage amount, a dosage frequency, a maximum number of doses deliverable from the drug administration device, and a dosage time.

For another example, the method can include communicating the data packet to a central computer system connected to the network. In at least some embodiments, the method can include communicating an administration signal from the central computer system to the drug administration device upon receipt of the data packet, and the drug administration device can be configured to permit administration of a drug only upon receipt of the administration signal. The method can also include the central computer system determining, based on the data packet, that at least one of a drug refill, emergency services, a user reminder prompt, and a variation in at least one of the parameters of the drug administration device is required.

For yet another example, the method can include communicating the data packet to a display device connected to the network. For still another example, the method can include the drug administration device determining, based on the data packet, that a drug refill or emergency services are required and communicating a status signal of the drug administration device to the central computer system. For another example, establishing communication can include the drug administration device scanning for available networks and external devices and establishing communication with the available networks and/or external devices. For yet another example, establishing communication can include establishing wireless communication between the drug administration device and the at least one of the network and the external device.

For another example, the ancillary data relating to the user parameter can include at least one of a location of a user, an environmental condition of a user, a psychological condition of a user, and a physiological condition of a user. In at least some embodiments, the location of the user, the physiological condition of the user, and/or the environmental condition of the user can be measured using at least one sensor of the external device. The location of the user, the physiological condition of the user, the psychological condition of the user, and/or the environmental condition of the user are measured continuously, at regular intervals, when requested by a user, or when requested by the drug administration device.

For still another example, the parameters of the drug administration device can include at least one of drug administration history, an amount of drug remaining in the drug administration device, drug composition data, and power consumption data. For another example, the drug administration data and the ancillary data can each include associated time values. For yet another example, the external device can be configured to record and/or influence a physiological condition of a user, and the external device can one or more of a user stress testing device; a user exercise device; a fitness wearable; a CPAP machine; a blood glucose monitor; and a heart rate monitor. For still another example,

For another example, the drug administration device can be configured to deliver a drug, and the drug can include at least one of infliximab, golimumab, ustekinumab, daratumumab, guselkumab, epoetin alfa, risperidone, esketamine, ketamine, and paliperidone palmitate.

In a second aspect, a drug administration system is provided that in one embodiment includes a drug administration device and at least one of a network and an external device. The drug administration device includes a housing that accommodates a drug holder and dispensing mechanism. The drug administration device further includes a communications interface configured to establish communication between the drug administration device and the at least one of the network and the external device and to receive ancillary data from the at least one of the network and the external device. The drug administration device further includes a processor configured to receive drug administration data having a first data format and relating to parameters of the drug administration device, filter the ancillary data such that ancillary data relating to a user parameter remains, convert at least the ancillary data relating to the user parameter into the first data format, and combine the drug administration data and the ancillary data relating to the user parameter to thereby generate a data packet.

The system can have any number of variations. For example, the processor can be further configured to adjust at least one operational parameter of the drug administration device based on the data packet. In at least some embodiments, the processor can be further configured to compare a data packet value to a reference data packet value and adjust the at least one operational parameter of the drug administration device based on the comparison to thereby adjust the at least one operation parameter, or the processor can be further configured to compare a plurality of data packet values to a plurality of reference data packet values and adjust the at least one operational parameter of the drug administration device based on the comparisons to thereby adjust the at least one operation parameter.

For another example, the communications interface can be further configured to communicate the data packet to a central computer system connected to the network. In at least some embodiments, the central computer system can be configured to communicate an administration signal to the drug administration device upon receipt of the data packet, and the processor can be configured to permit administration of a drug only upon receipt of the administration signal at the communications interface. The central computer system can be configured to determine, based on the data packet, that at least one of a drug refill, emergency services, a user reminder prompt, and a variation in at least one of the parameters of the drug administration device is required.

For still another example, the communications interface can be further configured to communicate the data packet to a display device connected to the network. For yet another example, the processor can be configured to determine, based on the data packet, that a drug refill or emergency services are required and to communicate a status signal of the drug administration device to a central computer system connected to the network. For another example, the communications interface can be configured to scan for available networks and external devices and to establish communication with the available networks and/or external devices. For yet another example, the communications interface can be configured to establish wireless communication between the drug administration device and the at least one of a network and the external device.

For still another example, the external device can include a sensor configured to measure a location of the user, a physiological condition of the user, and/or an environmental condition of the user. In at least some embodiments, the sensor can be configured to measure the location of the user, the physiological condition of the user, and/or the environmental condition of the user continuously, at regular intervals, when requested by a user, or when requested by the drug administration device.

For another example, the drug administration data and the ancillary data can each include associated time values. For yet another example, the external device can be configured to record and/or influence a physiological condition of a user, and the external device can include one or more of a user stress testing device, a user exercise device, a fitness wearable, a CPAP machine, a blood glucose monitor, and a heart rate monitor.

For still another example, the drug holder can be configured to hold a drug, the dispensing mechanism can be configured to dispense the drug, and the drug can include at least one of infliximab, golimumab, ustekinumab, daratumumab, guselkumab, epoetin alfa, risperidone, esketamine, ketamine, and paliperidone palmitate.

In another embodiment, a drug administration system includes a drug administration device and at least one external device including a sensor configured to measure user physiological data. The drug administration device includes a housing that accommodates a drug holder and dispensing mechanism. The drug administration device further includes a communications interface configured to scan for the at least one external device with which to establish communication, establish communication between the drug administration device and the at least one external device, and receive the user physiological data from the at least one external device. The drug administration further includes a processor in communication with the communications interface. The processor is configured to adjust at least one operational parameter of the drug administration device based on the user physiological data.

The system can vary in any number of ways. For example, the user physiological data can include a first user physiological parameter and a second user physiological parameter different from the first user physiological parameter, and the processor can be further configured to adjust the at least one operational parameter based on the first user physiological parameter and adjust the at least one operational parameter based on the second user physiological parameter. In at least some embodiments, the processor can be further configured to adjust the at least one operational parameter based on the first user physiological parameter if the second user physiological parameter is within a first range, and to adjust the at least one operational parameter based on the second user physiological parameter if the second user physiological parameter is within a second range. A limit of the first range can be equal to a threshold value, and the threshold value can be a boundary of the second range. When the second user physiological parameter is within the second range the processor can be further configured to determine an acceptable range of adjustment values of the at least one operational parameter based on the first user physiological parameter, determine a first adjustment value of the at least one operational parameter based on the second user physiological parameter, adjust the at least one operational parameter to the first adjustment value if the first adjustment value is within the acceptable range of adjustment values, and adjust the at least one operational parameter based on the first user physiological parameter if the first adjustment value is outside the acceptable range of adjustment values. The processor can be further configured to adjust the at least one operational parameter based on the first user physiological parameter and the second user physiological parameter when the first adjustment value is outside the acceptable range of adjustment values.

For another example, the processor can be further configured to adjust the at least one operational parameter based on the first user physiological parameter and the second user physiological parameter.

For yet another example, a first sensor can be configured to measure the first user physiological parameter, a second sensor can be configured to measure the second user physiological parameter, and either (1) the at least one external device can include a first external device including the first sensor and the second sensor, or (2) the at least one external device can include a first external device including the first sensor, and a second external device can include the second sensor.

For still another example, the processor can be further configured to identify a user physiological parameter that the user physiological data relates to. In at least some embodiments, the processor can be further configured to send a prompt to confirm that the at least one operation parameter should be adjusted based on the identified user physiological parameter to a display.

For another example, the communications interface can be configured to establish communication manually or automatically. For yet another example, the at least one external device can include a plurality of external devices. For still another example, the at least one operational parameter relates to at least one of a dosage amount, a dosage frequency, and a dosage time. For another example, the user physiological data can relate to at least one of blood concentration of dispensed drug, blood glucose level, and heart rate. For still another example, the at least one external device can be an implanted device. For yet another example, the external device can be an implantable device.

For another example, the drug holder can be configured to hold a drug, the dispensing mechanism can be configured to dispense the drug, and the drug can include at least one of infliximab, golimumab, ustekinumab, daratumumab, guselkumab, epoetin alfa, risperidone, esketamine, ketamine, and paliperidone palmitate.

In another embodiment, a drug delivery system includes a first drug administration device configured to administer a first drug and configured to operate in accordance with a first control program, and a second drug administration device configured to administer a second drug. The first drug administration device is configured to communicate with the second drug administration device. The first drug administration device includes a processor and a memory configured to communicate with the processor. The memory is configured to store at least one dosing parameter of the first control program, and the processor is configured to adjust the at least one dosing parameter in response to a communication received from the second drug administration device.

The system can have any number of variations. For example, the second drug administration device can be configured to operate in accordance with a second control program. For another example, the first drug administration device can be configured to receive the communication from the second drug administration device directly from the second drug administration device. For yet another example, the system can include a set of interaction parameters stored in the memory, and the interaction parameters can include dosing requirements based on the combination of the first drug and the second drug. For still another example, the processor can be configured to delay operation of the first drug administration device in accordance with the first control program based on the operation of the second drug administration device in accordance with the second control program.

For another example, the processor can be configured to avoid simultaneous operation of the first drug administration device in accordance with the first control program and operation of the second drug administration device in accordance with the second control program. In at least some embodiments, the processor can be configured to measure an elapsed time after an event associated with a control program, compare the elapsed time against an interaction parameter, and delay operation of the first drug administration device in accordance with the first control program if sufficient time has not passed.

For yet another example, the first drug administration device and the second drug administration device can share a housing. For still another example, the first drug administration device and the second drug administration device can share a component for drug delivery or communication. For yet another example, the first drug administration device and the second drug administration device can be configured to communicate wirelessly.

For another example, the system can include a physiological sensor configured to sense a body parameter. In at least some embodiments, the physiological sensor can be configured to communicate the sensed body parameter to the first drug administration device or the second drug administration device. The at least one dosing parameter of the first control program can be adjusted in response to the sensed body parameter. The processor can be configured to delay operation of the first drug administration device in accordance with the first control program in response to the sensed body parameter.

For yet another example, the at least one dosing parameter of the first control program can be configured to be updated in response to a manual input. For another example, the first drug administration device can be configured to notify a user when the first drug administration device fails to receive a communication from the second drug administration device. For yet another example, the dosing parameters of the first control program can be configured to be reset to default dosing parameters when the first drug administration device fails to receive a communication from the second drug administration device.

For still another example, the at least one dosing parameter of the first control program can include any of duration of drug administration, dosage of medication, frequency of drug administration, and time of administration.

For another example, the first drug and the second drug can each include at least one of infliximab, golimumab, ustekinumab, daratumumab, guselkumab, epoetin alfa, risperidone, esketamine, ketamine, and paliperidone palmitate.

In another embodiment, a drug delivery system includes a first drug administration device configured to administer a first drug and configured to operate in accordance with a first control program, a second drug administration device configured to administer a second drug, and an external device. The first drug administration device is configured to communicate with the second drug administration device. The external device includes a processor and a memory configured to communicate with the processor and configured to store at least one dosing parameter of the first control program. The processor is configured to adjust the at least one dosing parameter in response to a communication received from the second drug administration device.

The system can have any number of variations. For example, the second drug administration device can be configured to operate in accordance with a second control program. In at least some embodiments, the external device can be configured to communicate with the first drug administration device and the second drug administration device. One of the first drug administration device or the external device can be a primary controller, and the primary controller can be configured to operate the first drug administration device in accordance with the first control program and operate the second drug administration device in accordance with the second control program.

For another example, the first drug administration device can be configured to receive the communication from the second drug administration device indirectly from the second drug administration device via the external device. For yet another example, the system can include a set of interaction parameters stored in the memory, and the interaction parameters can include dosing requirements based on the combination of the first drug and the second drug. For still another example, the processor can be configured to delay operation of the first drug administration device in accordance with the first control program based on the operation of the second drug administration device in accordance with the second control program.

For another example, the processor can be configured to avoid simultaneous operation of the first drug administration device in accordance with the first control program and operation of the second drug administration device in accordance with the second control program. In at least some embodiments, the processor can be configured to measure an elapsed time after an event associated with a control program, compare the elapsed time against an interaction parameter, and delay operation of the first drug administration device in accordance with the first control program if sufficient time has not passed.

For yet another example, the first drug administration device and the second drug administration device can share a housing. For still another example, the first drug administration device and the second drug administration device can share a component for drug delivery or communication. For yet another example, the first drug administration device and the second drug administration device can be configured to communicate wirelessly.

For another example, the system can include a physiological sensor configured to sense a body parameter. In at least some embodiments, the physiological sensor can be configured to communicate the sensed body parameter to the first drug administration device, the second drug administration device, or the external device. The at least one dosing parameter of the first control program can be adjusted in response to the sensed body parameter. The processor can be configured to delay operation of the first drug administration device in accordance with the first control program in response to the sensed body parameter.

For yet another example, the at least one dosing parameter of the first control program can be configured to be updated in response to a manual input. For another example, the first drug administration device can be configured to notify a user when the first drug administration device fails to receive a communication from the second drug administration device or the external device. For yet another example, the dosing parameters of the first control program can be configured to be reset to default dosing parameters when the first drug administration device fails to receive a communication from the second drug administration device or the external device.

For still another example, the at least one dosing parameter of the first control program can include any of duration of drug administration, dosage of medication, frequency of drug administration, and time of administration.

For another example, the first drug and the second drug can each include at least one of infliximab, golimumab, ustekinumab, daratumumab, guselkumab, epoetin alfa, risperidone, esketamine, ketamine, and paliperidone palmitate.

In a third aspect, a drug administration device is provided that in one embodiment includes a housing that accommodates a drug holder and dispensing mechanism. The drug administration device further includes a communications interface configured to establish communication between the drug administration device and at least one of a network and an external device and to receive ancillary data from the at least one of the network and the external device. The drug administration device further includes a processor configured to receive drug administration data having a first data format and relating to parameters of the drug administration device, filter the ancillary data such that ancillary data relating to a user parameter remains, convert at least the ancillary data relating to the user parameter into the first data format, and combine the drug administration data and the ancillary data relating to the user parameter to thereby generate a data packet.

The device can vary in any number of ways. For example, the processor can be further configured to adjust at least one operational parameter of the drug administration device based on the data packet. In at least some embodiments, the processor can be further configured to compare a data packet value to a reference data packet value and adjust the at least one operational parameter of the drug administration device based on the comparison to thereby adjust the at least one operation parameter, or the processor can be further configured to compare a plurality of data packet values to a plurality of reference data packet values and adjust the at least one operational parameter of the drug administration device based on the comparisons to thereby adjust the at least one operation parameter.

For another example, the communications interface can be further configured to communicate the data packet to a central computer system connected to the network. In at least some embodiments, the processor can be configured to permit administration of a drug only upon receipt of an administration signal at the communications interface from the central computer system.

For yet another example, the communications interface can be further configured to communicate the data packet to a display device connected to the network. For still another example, the processor can be configured to determine, based on the data packet, that a drug refill or emergency services are required and to communicate a status signal of the drug administration device to a central computer system connected to the network. For another example, the communications interface can be configured to establish wireless communication between the drug administration device and the at least one of a network and the external device. For still another example, the drug administration data and the ancillary data can each include associated time values.

For another example, the drug holder can be configured to hold a drug, the dispensing mechanism can be configured to dispense the drug, and the drug can include at least one of infliximab, golimumab, ustekinumab, daratumumab, guselkumab, epoetin alfa, risperidone, esketamine, ketamine, and paliperidone palmitate.

In another embodiment, a drug administration device includes a housing that accommodates a drug holder and a dispensing mechanism. The device also includes a communications interface configured to scan for at least one external device with which to establish communication, establish communication between the drug administration device and the at least one external device, and receive user physiological data from the at least one external device. The device also includes a processor in communication with the communications interface, and the processor is configured to adjust at least one operational parameter of the drug administration device based on the user physiological data.

The device can have any number of variations. For example, the user physiological data can include a first user physiological parameter and a second user physiological parameter different from the first user physiological parameter, and the processor can be further configured to adjust the at least one operational parameter based on the first user physiological parameter and to adjust the at least one operational parameter based on the second user physiological parameter. In at least some embodiments, the processor can be further configured to adjust the at least one operational parameter based on the first user physiological parameter if the second user physiological parameter is within a first range, and to adjust the at least one operational parameter based on the second user physiological parameter if the second user physiological parameter is within a second range. A limit of the first range can be equal to a threshold value, and the threshold value can be a boundary of the second range. When the second user physiological parameter is within the second range the processor can be further configured to determine an acceptable range of adjustment values of the at least one operational parameter based on the first user physiological parameter, determine a first adjustment value of the at least one operational parameter based on the second user physiological parameter, adjust the at least one operational parameter to the first adjustment value if the first adjustment value is within the acceptable range of adjustment values, and adjust the at least one operational parameter based on the first user physiological parameter, and optionally the second user physiological parameter, if the first adjustment value is outside the acceptable range of adjustment values. The processor can be further configured to adjust the at least one operational parameter based on the first user physiological parameter and the second user physiological parameter when the first adjustment value is outside the acceptable range of adjustment values.

For yet another example, the processor can be further configured to adjust the at least one operational parameter based on the first user physiological parameter and the second user physiological parameter.

For another example, a first sensor can be configured to measure the first user physiological parameter, a second sensor can be configured to measure the second user physiological parameter, and either (1) the at least one external device can include a first external device comprising the first sensor and the second sensor, or (2) the at least one external device can include a first external device including the first sensor, and a second external device including the second sensor.

For yet another example, the processor can be further configured to identify a user physiological parameter that the user physiological data relates to. In at least some embodiments, the processor can be further configured to send a prompt to confirm that the at least one operation parameter should be adjusted based on the identified user physiological parameter to a display.

For still another example, the communications interface can be configured to establish communication manually or automatically. For another example, the at least one external device can include a plurality of external devices. For yet another example, the at least one operational parameter can relate to at least one of a dosage amount, a dosage frequency, and a dosage time. For still another example, the user physiological data can relate to at least one of blood concentration of dispensed drug, blood glucose level, and heart rate.

For another example, the drug holder can be configured to hold a drug, the dispensing mechanism can be configured to dispense the drug, and the drug can include at least one of infliximab, golimumab, ustekinumab, daratumumab, guselkumab, epoetin alfa, risperidone, esketamine, ketamine, and paliperidone palmitate.

In another embodiment, a drug administration device includes a control program for administering a drug, the drug administration device is configured to update the control program in response to an update request sent from a network or an external device, the drug administration device includes a processor and a memory in communication with the processor, the drug administration device is configured to require a local authorization to authorize the update request, the processor is configured to update the control program based on data from the network or the external device if the update request is authorized on the drug administration device, and the processor is configured to prevent the control program from updating if the update request is not authorized on the drug administration device.

The device can vary in any number of ways. For example, the drug administration device can include a user interface device. In at least some embodiments, the local authorization can be received via the user interface device.

For another example, the drug administration device can be configured to confirm the identity of a digital signature received from the network or the external device. For yet another example, the processor of the drug administration device can be configured to store a digital characteristic of the communication link or the update request in the memory of the drug administration device if the update request is not authorized within the predetermined time frame. For still another example, the processor can be configured to place the drug administration device into a locked state in response to a security protocol, and the drug administration device can be prevented from any wireless communication in the locked state. For another example, in response to a security protocol, the drug administration device can be configured to communicate an error message to an indicator or to the user interface device, to notify a user.

For yet another example, the drug administration device can be configured to deliver a drug, and the drug can include at least one of infliximab, golimumab, ustekinumab, daratumumab, guselkumab, epoetin alfa, risperidone, esketamine, ketamine, and paliperidone palmitate.

In another embodiment, a drug administration device is configured to operate in accordance with a control program, and the drug administration device includes a processor and a memory configured to communicate with the processor. The memory is configured to store at least one dosing parameter of the control program, the drug administration device is configured to communicate with a second drug administration device, and the drug administration device is configured to adjust at least one dosing parameter of the control program in response to a communication received from the second drug administration device.

The drug administration device can have any number of variations. For example, the drug administration device can be configured to communicate with an external device. For another example, the drug administration device can be configured to receive the communication from the second drug administration device indirectly via an external device. For yet another example, the processor can be configured to delay operation of the drug administration device in accordance with the control program. For still another example, the processor can be configured to avoid simultaneous operation of the drug administration device in accordance with the control program and operation of the second drug administration device in accordance with a second control program. For another example, the drug administration device can be configured to communicate wirelessly to the second drug administration device.

For yet another example, the drug administration device can be configured to deliver a drug, and the drug can include at least one of infliximab, golimumab, ustekinumab, daratumumab, guselkumab, epoetin alfa, risperidone, esketamine, ketamine, and paliperidone palmitate.

In a fourth aspect, a method of adjusting at least one operational parameter of a drug administration device is provided that in one embodiment includes scanning for at least one external device with the drug administration device, establishing communication between the drug administration device and the at least one external device, Receiving user physiological data from the at least one external device at the drug administration device, and adjusting at least one operational parameter of the drug administration device based on the user physiological data.

The method can have any number of variations. For example, the user physiological data can include a first user physiological parameter and a second user physiological parameter different from the first user physiological parameter, and adjusting the at least one operational parameter can include adjusting the at least one operational parameter based on the first user physiological parameter or adjusting the at least one operational parameter based on the second user physiological parameter. In at least some embodiments, the method can further include adjusting the at least one operational parameter based on the first user physiological parameter if the second user physiological parameter is within a first range, and adjusting the at least one operational parameter based on the second user physiological parameter if the second user physiological parameter is within a second range. A limit of the first range can be equal to a threshold value, and the threshold value can be a boundary of the second range. When the second user physiological parameter is within the second range, the method can further include determining an acceptable range of adjustment values of the at least one operational parameter based on the first user physiological parameter, determining a first adjustment value of the at least one operational parameter based on the second user physiological parameter, adjusting the at least one operational parameter to the first adjustment value if the first adjustment value is within the acceptable range of adjustment values, and adjusting the at least one operational parameter based on the first user physiological parameter if the first adjustment value is outside the acceptable range of adjustment values. Adjusting the at least one operational parameter when the first adjustment value is outside the acceptable range of adjustment values can further include adjusting the at least one operational parameter based on the first user physiological parameter and the second user physiological parameter.

For another example, adjusting the at least one operational parameter can further include adjusting the at least one operational parameter based on the first user physiological parameter and the second user physiological parameter. For yet another example, the first user physiological parameter and the second user physiological parameters can be received from the same external device or from separate external devices.

For still another example, the method can also include identifying a user physiological parameter that the user physiological data relates to. In at least some embodiments, the method can also include prompting a user to confirm that the at least one operation parameter should be adjusted based on the identified user physiological parameter.

For yet another example, communication can be established manually or automatically. For another example, the at least one external device can include a plurality of external devices. For still another example, the at least one operational parameter can relate to at least one of a dosage amount, a dosage frequency, and a dosage time. For another example, the user physiological data can relate to at least one of blood concentration of dispensed drug, blood glucose level, and heart rate. For yet another example, the at least one external device can be an implanted device.

For another example, the drug administration device can be configured to deliver a drug, and the drug can include at least one of infliximab, golimumab, ustekinumab, daratumumab, guselkumab, epoetin alfa, risperidone, esketamine, ketamine, and paliperidone palmitate.

In a fifth aspect, a method of assessing whether to update a control program of a drug administration device that in one embodiment includes establishing a communication link between the drug administration device and a network or an external device; sending an update request from the network or the external device to the drug administration device; confirming, on the drug administration device, whether the update request is authorized; and, if the update request is authorized within a predetermined time frame, updating one or more parameters of the control program of the drug administration device; or, if the update request is not authorized within the predetermined time frame, preventing the drug administration device from updating any parameters of the control program.

The method can vary in any number of ways. For example, confirming whether an update request is authorized can include confirming whether an input from a user has occurred on the drug administration device. In at least some embodiments, the method can further include prompting the user to provide an input prior to confirming whether the input from the user has occurred on the drug administration device.

For another example, confirming whether an update request is authorized can include confirming whether the external device or network is an acceptable external device or network. For yet another example, establishing the communication link can include communicating a digital signature from the network or the external device to the drug administration device, and confirming an identity of the digital signature is valid on the drug administration device. For still another example, the method can include storing a digital characteristic of the communication link or the update request if the update request is not authorized within the predetermined time frame. The method can also include comparing the stored digital characteristic against a pre-defined list of known digital characteristics stored in the drug administration device.

For yet another example, the method can include implementing a first security protocol if the update request is not authorized within the predetermined time frame. In at least some embodiments, the method can also include comparing the stored digital characteristic against a pre-defined list of known digital characteristics stored in the drug administration device, and the first security protocol can include implementing a counter-measure if the stored digital characteristic of the communication link or the update request matches a known digital characteristic in the pre-defined list. The first security protocol can include notifying an external system. The first security protocol can include, after a predetermined number of failed update authorizations, locking the drug administration device into a locked state, and the drug administration device in the locked state can be prevented from communicating remotely. The method can also include requiring an input from a user to be received on the drug administration device to resume remote communication when the drug administration device is in the locked state.

For another example, the method can include requiring a second update authorization to be confirmed on either the external device or a control device connected to the network within a second predetermined time frame. For yet another example, the one or more parameters of the control program of the drug administration device can be stored in an electronic memory. For still another example, the one or more parameters of the control program can include one of drug dosage, frequency of drug administration, and duration of drug administration. For another example, the external device can be an internet-connected device. For still another example, the external device can be a smart phone.

For yet another example, the drug administration device can be configured to deliver a drug, and the drug can include at least one of infliximab, golimumab, ustekinumab, daratumumab, guselkumab, epoetin alfa, risperidone, esketamine, ketamine, and paliperidone palmitate.

In a sixth aspect, a method of administering a drug with a drug administration device is provided that in one embodiment include receiving a communication on the drug administration device from a second drug administration device, determining whether to adjust a dosing parameter of a first control program on the drug administration device as a result of the communication, adjusting the dosing parameter of the first control program if it is determined to adjust the dosing parameter, and administering the drug by the drug administration device in accordance with the first control program.

The method can vary in any number of ways. For example, the drug administration device can receive the communication from the second drug administration device via an external device. For another example, the determination of whether to adjust the dosing parameter can be based on an interaction parameter, stored in the dug administration device, and an elapsed time. For yet another example, the drug administration device can receive the communication from the second drug administration device wirelessly.

For still another example, the method can include adjusting one or more dosing parameters of the first control program, and the one or more dosing parameters can be adjusted based on a sensed body parameter sensed by a physiological sensor. In at least some embodiments, the dosing parameters can be adjusted when the sensed body parameter exceeds a predetermined threshold.

For another example, the drug administration device can be configured to deliver a drug, and the drug can include at least one of infliximab, golimumab, ustekinumab, daratumumab, guselkumab, epoetin alfa, risperidone, esketamine, ketamine, and paliperidone palmitate.

### BRIEF DESCRIPTION OF DRAWINGS

The present invention is described by way of reference to the accompanying figures which are as follows:

FIG. 1 is a schematic view of a first type of drug administration device, namely an autoinjector;

FIG. 2 is a schematic view of a second type of drug administration device, namely an infusion pump;

FIG. 3 is a schematic view of a third type of drug administration device, namely an inhaler;

FIG. 4 is a schematic view of a fourth type of drug administration device, namely a nasal spray device;

FIG. 5A is a schematic view of a general drug administration device;

FIG. 5B is a schematic view of a universal drug administration device;

FIG. 6 is a schematic view of a housing for a dosage form;

FIG. 7 is a schematic view of one embodiment of a communication network system with which the drug administration devices and housing can operate;

FIG. 8 is a schematic view of one embodiment of a computer system with which the drug administration devices and housing can operate;

FIG. 9 is a schematic view of one embodiment of a drug administration system

FIG. 10 is a flow diagram of operation of one embodiment of the system of FIG. 9;

FIG. 10A is a perspective view of one embodiment of a smartphone;

FIG. 10B is a perspective view of one embodiment of a wearable injection device worn on an arm of a patient and configured to communicate wirelessly;

FIG. 10C is a perspective view of one embodiment of an autoinjector configured to communicate wirelessly;

FIG. 11 is a schematic view of one embodiment of a drug administration system;

FIG. 12 is a flow diagram of one embodiment of operation of the system of FIG. 11;

FIG. 13 is a flow diagram of a further aspect of an embodiment of operation of a drug administration system;

FIG. 14 is a flow diagram of a yet further aspect of an embodiment of operation of a drug administration system;

FIG. 15 is a schematic view of one embodiment of a drug administration system for remote communication to a drug administration device;

FIG. 16 is a flowchart of one embodiment for transferring data to a drug administration device;

FIG. 17 is a flowchart of one embodiment for establishing a communication link between a network or an external device and a drug administration device;

FIG. 18 is a flowchart of one embodiment for updating a control program of a drug administration device with an update authorization;

FIG. 19 is a schematic view of one embodiment of a drug administration device configured for wireless communication;

FIG. 20 is a schematic view of one embodiment of two drug administration devices in wireless communication;

FIG. 21 is a schematic view of one embodiment of two drug administration devices in a wired communication;

FIG. 22 is a schematic view of one embodiment of two drug administration devices in a single housing, sharing a power supply and a processor;

FIG. 23 is a timing diagram of one embodiment of first and second control programs, wherein one of the control programs is configured to be delayed in response to the other control program;

FIG. 24 is a schematic view of one embodiment of two drug administration devices and an external device;

FIG. 25 is a schematic view of one embodiment of two drug administration devices, an external device, and a physiological sensor; and

FIG. 26 is a timing diagram showing one embodiment of delay of a control program until a measured parameter falls below a threshold.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Examples of various types of drug administration devices, namely: an autoinjector 100, an infusion pump 200, an inhaler 300, and a nasal spray device 400, are described below with reference to the hereinbefore referenced figures.

### Autoinjector

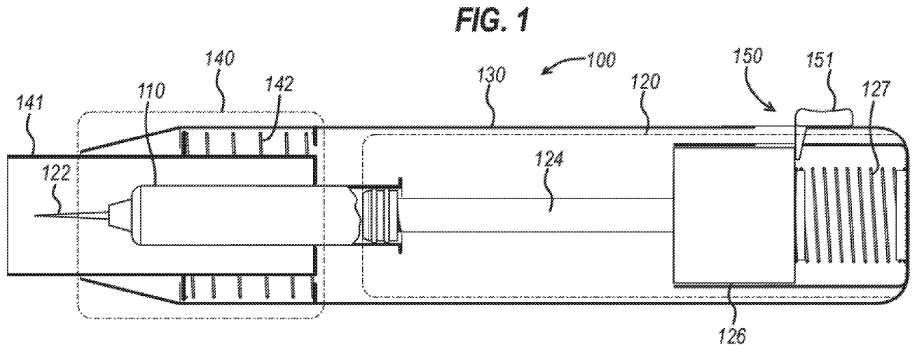

FIG. 1 is a schematic exemplary view of a first type of drug delivery device, namely an injection device, in this example an autoinjector 100, useable with embodiments described herein. The autoinjector 100 comprises a drug holder 110 which retains a drug to be dispensed and a dispensing mechanism 120 which is configured to dispense a drug from the drug holder 110 so that it can be administered to a patient. The drug holder 110 is typically in the form of a container which contains the drug, for example it may be provided in the form of a syringe or a vial, or be any other suitable container which can hold the drug. The autoinjector 100 comprises a discharge nozzle 122, for example a needle of a syringe, which is provided at a distal end of the drug holder 110. The dispensing mechanism 120 comprises a drive element 124, which itself may also comprise a piston and/or a piston rod, and drive mechanism 126. The dispensing mechanism 120 is located proximal to the end of the drug holder 110 and towards the proximal end of the autoinjector 100.

The autoinjector 100 comprises a housing 130 which contains the drug holder 110, drive element 124 and drive mechanism 126 within the body of the housing 130, as well as containing the discharge nozzle 122, which, prior to injection, would typically be contained fully within the housing, but which would extend out of the housing 130 during an injection sequence to deliver the drug. The dispensing mechanism 120 is arranged so that the drive element 124 is advanced through the drug holder 110 in order to dispense the drug through the discharge nozzle 122, thereby allowing the autoinjector to administer a drug retained in drug holder 110 to a patient. In some instances, a user may advance the drive element 124 through the drug holder 110 manually. In other instances, the drive mechanism 126 may include a stored energy source 127 which advances the drive element 124 without user assistance. The stored energy source 127 may include a resilient biasing member such as a spring, or a pressurized gas, or electronically powered motor and/or gearbox.

The autoinjector 100 includes a dispensing mechanism protection mechanism 140. The dispensing mechanism protection mechanism 140 typically has two functions. Firstly, the dispensing mechanism protection mechanism 140 can function to prevent access to the discharge nozzle 122 prior to and after injection. Secondly, the autoinjector 100 can function, such that when put into an activated state, e.g., the dispensing mechanism protection mechanism 140 is moved to an unlocked position, the dispensing mechanism 120 can be activated.

The protection mechanism 140 covers at least a part of the discharge nozzle 122 when the drug holder 110 is in its retracted position proximally within the housing 130. This is to impede contact between the discharge nozzle 122 and a user. Alternatively, or in addition, the protection mechanism 140 is itself configured to retract proximally to expose the discharge nozzle 122 so that it can be brought into contact with a patient. The protection mechanism 140 comprises a shield member 141 and return spring 142. Return spring 142 acts to extend the shield member 141 from the housing 130, thereby covering the discharge nozzle 122 when no force is applied to the distal end of the protection mechanism 140. If a user applies a force to the shield member 141 against the action of the return spring 142 to overcome the bias of the return spring 142, the shield member 141 retracts within the housing 130, thereby exposing the discharge nozzle 122. The protection mechanism 140 may alternatively, or in addition, comprise an extension mechanism (not shown) for extending the discharge nozzle 122 beyond the housing 130, and may further comprise a retracting mechanism (not shown) for retracting the discharge nozzle 122 within the housing 130. The protection mechanism 140 may alternatively, or in addition, comprise a housing cap and/or discharge nozzle boot, which can be attached to the autoinjector 100. Removal of the housing cap would typically also remove the discharge nozzle boot from the discharge nozzle 122.

The autoinjector 100 also includes a trigger 150. The trigger 150 comprises a trigger button 151 which is located on an external surface of the housing 130 so that it is accessible by a user of the autoinjector 100. When the trigger 150 is pressed by a user, it acts to release the drive mechanism 126 so that, via the drive element 124, the drug is then driven out of the drug holder 110 via the discharge nozzle 122.

The trigger 150 may also cooperate with the shield member 141 in such a way that the trigger 150 is prevented from being activated until the shield member 141 has been retracted proximally sufficiently into the housing 130 into an unlocked position, for example by pushing a distal end of the shield member 141 against the skin of a patient. When this has been done, the trigger 150 becomes unlocked, and the autoinjector 100 is activated such that the trigger 150 can be depressed and the injection and/or drug delivery sequence is then initiated. Alternatively, retraction of the shield member 141 alone in a proximal direction into the housing 130 can act to activate the drive mechanism 126 and initiate the injection and/or drug delivery sequence. In this way, the autoinjector 100 has device operation prevention mechanism which prevents dispensing of the drug by, for example, preventing accidental release of the dispensing mechanism 120 and/or accidental actuation of the trigger 150.

While the foregoing description relates to one example of an autoinjector, this example is presented purely for illustration, the present invention is not limited solely to such an autoinjector. A person skilled in the art understands that various modifications to the described autoinjector may be implemented within the scope of the present disclosure.

Autoinjectors of the present disclosure can be used to administer any of a variety of drugs, such as any of epinephrine, Rebif, Enbrel, Aranesp, atropine, pralidoxime chloride, and diazepam.

### Infusion Pump

In other circumstances, patients can require precise, continuous delivery of medication or medication delivery on a regular or frequent basis at set periodic intervals. Infusion pumps can provide such controlled drug infusion, by facilitating the administering of the drug at a precise rate that keeps the drug concentration within a therapeutic margin, without requiring frequent attention by a healthcare professional or the patient.

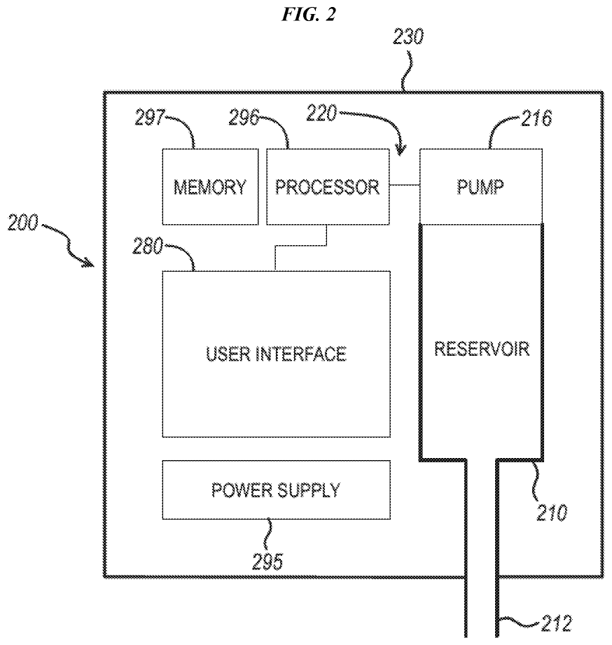

FIG. 2 is a schematic exemplary view of a second type of drug delivery device, namely an infusion pump 200, useable with the embodiments described herein. The infusion pump 200 comprises a drug holder 210 in the form of a reservoir for containing a drug to be delivered, and a dispensing mechanism 220 comprising a pump 216 adapted to dispense a drug contained in the reservoir, so that the drug can be delivered to a patient. These components of the infusion pump are located within housing 230. The dispensing mechanism 220 further comprises an infusion line 212. The drug is delivered from the reservoir upon actuation of the pump 216 via the infusion line 212, which may take the form of a cannula. The pump 216 may take the form of an elastomeric pump, a peristaltic pump, an osmotic pump, or a motor-controlled piston in a syringe. Typically, the drug is delivered intravenously, although subcutaneous, arterial and epidural infusions may also be used.

Infusion pumps of the present disclosure can be used to administer any of a variety of drugs, such as any of insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, and zoledronic acid.

The infusion pump 200 further comprises control circuitry, for example a processor 296 in addition to a memory 297 and a user interface 280, which together provide a triggering mechanism and/or dosage selector for the pump 200. The user interface 280 may be implemented by a display screen located on the housing 230 of the infusion pump 200. The control circuitry and user interface 280 can be located within the housing 230, or external thereto and communicate via a wired or wireless interface with the pump 216 to control its operation.

Actuation of the pump 216 is controlled by the processor 296 which is in communication with the pump 216 for controlling the pump's operation. The processor 296 may be programmed by a user (e.g., patient or healthcare professional), via a user interface 280. This enables the infusion pump 200 to deliver the drug to a patient in a controlled manner. The user can enter parameters, such as infusion duration and delivery rate. The delivery rate may be set by the user to a constant infusion rate or as set intervals for periodic delivery, typically within pre-programmed limits. The programmed parameters for controlling the pump 216 are stored in and retrieved from the memory 297 which is in communication with the processor 296. The user interface 280 may take the form of a touch screen or a keypad.

A power supply 295 provides power to the pump 216, and may take the form of an energy source which is integral to the pump 216 and/or a mechanism for connecting the pump 216 to an external source of power.

The infusion pump 200 may take on a variety of different physical forms depending on its designated use. It may be a stationary, non-portable device, e.g., for use at a patient's bedside, or it may be an ambulatory infusion pump which is designed to be portable or wearable. An integral power supply 295 is particularly beneficial for ambulatory infusion pumps.

While the foregoing description relates to one example of an infusion pump, this example is provided purely for illustration. The present disclosure is not limited to such an infusion pump. A person skilled in the art understands that various modifications to the described infusion pump may be implemented within the scope of the present disclosure. For example, the processor may be pre-programmed, such that it is not necessary for the infusion pump to include a user interface.

### Inhaler

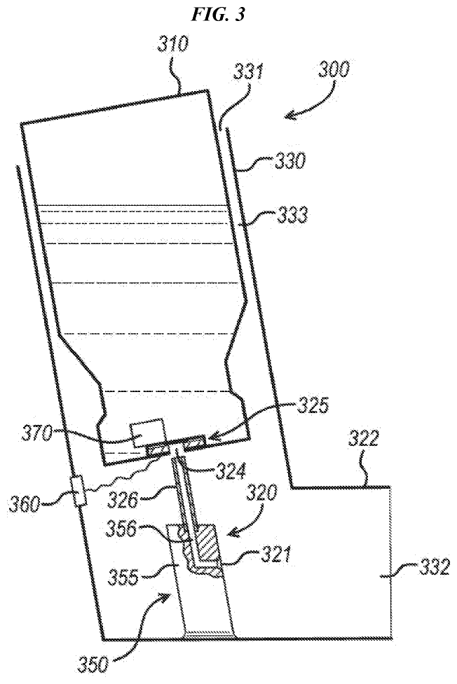

FIG. 3 is a schematic view of a third type of drug administration device, namely an inhaler 300. Inhaler 300 includes a drug holder 310 in the form of a canister. The drug holder 310 contains a drug that would typically be in solution or suspension with a suitable carrier liquid. The inhaler 300 further comprises a dispensing mechanism 320, which includes a pressurized gas for pressurizing the drug holder 310, a valve 325 and nozzle 321. The valve 325 forms an outlet of the drug holder 310. The valve 325 comprises a narrow opening 324 formed in the drug holder 310 and a movable element 326 that controls the opening 324. When the movable element 326 is in a resting position, the valve 325 is in a closed or unactuated state in which the opening 324 is closed and the drug holder 310 is sealed. When the movable element 326 is actuated from the resting position to an actuated position, the valve 325 is actuated into an open state in which the opening 324 is open. Actuation of the movable element 326 from the resting position to the actuated position comprises moving the movable element 326 into the drug holder 310. The movable element 326 is resiliently biased into the resting position. In the open state of the valve 325, the pressurized gas propels the drug in solution or suspension with the suitable liquid out of the drug holder 310 through the opening 324 at high speed. The high speed passage of the liquid through the narrow opening 324 causes the liquid to be atomized, that is, to transform from a bulk liquid into a mist of fine droplets of liquid and/or into a gas cloud. A patient may inhale the mist of fine droplets and/or the gas cloud into a respiratory passage. Hence, the inhaler 300 is capable of delivering a drug retained within the drug holder 310 into a respiratory passage of a patient.

The drug holder 310 is removably held within a housing 330 of the inhaler 300. A passage 333 formed in the housing 330 connects a first opening 331 in the housing 330 and a second opening 332 in the housing 330. The drug holder 310 is received within the passage 333. The drug holder 310 is slidably insertable through the first opening 331 of the housing 330 into the passage 333. The second opening 332 of the housing 330 forms a mouthpiece 322 configured to be placed in a patient's mouth or a nosepiece configured to be placed in a patient's nostril, or a mask configured to be placed over the patient's mouth and nose. The drug holder 310, the first opening 331 and the passage 333 are sized such that air can flow through the passage 333, around the drug holder 310, between the first opening 331 and the second opening 332. The inhaler 300 may be provided with a dispensing mechanism protection mechanism 140 in the form of a cap (not shown) which can be fitted to the mouthpiece 322.

Inhaler 300 further comprises a trigger 350 including a valve actuation feature 355 configured to actuate the valve 325 when the trigger 350 is activated. The valve actuation feature 355 is a projection of the housing 330 into the passage 333. The drug holder 310 is slidably movable within the passage 333 from a first position into a second position. In the first position, an end of the movable element 326 in the resting position abuts the valve actuation feature 355. In the second position, the drug holder 310 can be displaced towards the valve actuation feature 355 such that the valve actuation feature 355 moves the movable element 326 into the drug holder 310 to actuate the valve 325 into the open state. The user's hand provides the necessary force to move the drug holder 310 from the first position to the second position against the resiliently biased movable element 326. The valve actuation feature 355 includes an inlet 356, which is connected to the nozzle 321. The inlet 356 of the valve actuation feature 355 is sized and positioned to couple to the opening 324 of the valve 325 such that the ejected mist of droplets and/or gas cloud can enter the inlet 356 and exit from the nozzle 321 into the passage 333. The nozzle 321 assists in the atomization of the bulk liquid into the mist of droplets and/or gas cloud.

The valve 325 provides a metering mechanism 370. The metering mechanism 370 is configured to close the valve after a measured amount of liquid, and therefore, drug, has passed through the opening 324. This allows a controlled dose to be administered to the patient. Typically, the measured amount of liquid is pre-set, however, the inhaler 300 may be equipped with a dosage selector 360 that is user operable to change the defined amount of liquid.

While the foregoing description relates to one particular example of an inhaler, this example is purely illustrative. The description should not be seen as limited only to such an inhaler. A person skilled in the art understands that numerous other types of inhaler and nebulizers may be used with the present disclosure. For example, the drug may be in a powdered form, the drug may be in liquid form, or the drug may be atomized by other forms of dispensing mechanism 320 including ultrasonic vibration, compressed gas, a vibrating mesh, or a heat source.

The inhalers of the present disclosure can be used to administer any of a variety of drugs, such as any of mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

### Nasal Spray Device

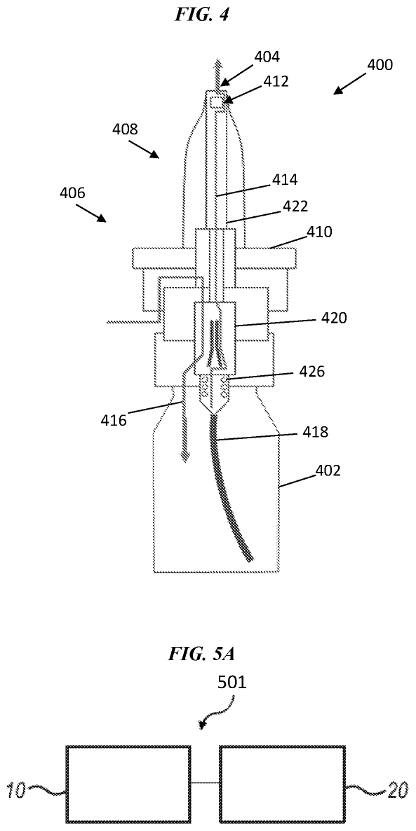

FIG. 4 is a schematic view of a fourth type of drug administration device, namely a nasal spray device 400. The nasal spray device 400 is configured to expel a drug into a nose of a patient. The nasal spray device 400 includes a drug holder 402 configured to contain a drug therein for delivery from the device 400 to a patient. The drug holder 102 can have a variety of configurations, such as a bottle reservoir, a cartridge, a vial (as in this illustrated embodiment), a blow-fill-seal (BFS) capsule, a blister pack, etc. In an exemplary embodiment, the drug holder 402 is a vial. An exemplary vial is formed of one or more materials, e.g., glass, polymer(s), etc. In some embodiments, a vial can be formed of glass. In other embodiments, a vial can be formed of one or more polymers. In yet other embodiments, different portions of a vial can be formed of different materials. An exemplary vial can include a variety of features to facilitate sealing and storing a drug therein, as described herein and illustrated in the drawings. However, a person skilled in the art will appreciate that the vials can include only some of these features and/or can include a variety of other features known in the art. The vials described herein are merely intended to represent certain exemplary embodiments.

An opening 404 of the nasal spray device 400 through which the drug exits the nasal spray device 400 is formed in in a dispensing head 406 of the nasal spray device 400 in a tip 408 of the dispensing head 406. The tip 408 is configured to be inserted into a nostril of a patient. In an exemplary embodiment, the tip 408 is configured to be inserted into a first nostril of the patient during a first stage of operation of the nasal spray device 400 and into a second nostril of the patient during a second stage of operation of the nasal spray device 400. The first and second stages of operation involve two separate actuations of the nasal spray device 400, a first actuation corresponding to a first dose of the drug being delivered and a second actuation corresponding to a second dose of the drug being delivered. In some embodiments, the nasal spray device 400 is configured to be actuated only once to deliver one nasal spray. In some embodiments, the nasal spray device 400 is configured to be actuated three or more times to deliver three or more nasal sprays, e.g., four, five, six, seven, eight, nine, ten, etc.

The dispensing head 406 includes a depth guide 410 configured to contact skin of the patient between the patient's first and second nostrils, such that a longitudinal axis of the dispensing head 406 is substantially aligned with a longitudinal axis of the nostril in which the tip 408 is inserted. A person skilled in the art will appreciate that the longitudinal axes may not be precisely aligned but nevertheless be considered to be substantially aligned due to any number of factors, such as manufacturing tolerances and sensitivity of measurement equipment.

In an exemplary embodiment, as in FIG. 4, the dispensing head 406 has a tapered shape in which the dispensing head 406 has a smaller diameter at its distal end than at its proximal end where the opening 404 is located. The opening 404 having a relatively small diameter facilitates spray of the drug out of the opening 404, as will be appreciated by a person skilled in the art. A spray chamber 412 through which the drug is configured to pass before exiting the opening 404 is located within a proximal portion of the tapered dispensing head 406, distal to the opening 404. When the drug passes through the spray chamber 412 at speed, the spray chamber 412 facilitates production of a fine mist that exits through the opening 404 with a consistent spray pattern. Arrow 414 in FIG. 4 illustrates a path of travel of the drug from the drug holder 402 and out of the opening 404.

In some embodiments, the dispensing head 406 can include two tips 408 each having an opening 404 therein such that the nasal spray device 400 is configured to simultaneously deliver doses of drug into two nostrils in response to a single actuation.

The dispensing head 406 is configured to be pushed toward the drug holder 402, e.g., depressed by a user pushing down on the depth guide 410, to actuate the nasal spray device 400. In other words, the dispensing head 406 is configured as an actuator to be actuated to drive the drug from the drug holder 402 and out of the nasal spray device 400. In an exemplary embodiment, the nasal spray device 400 is configured to be self-administered such that the user who actuates the nasal spray device 400 is the patient receiving the drug from the nasal spray device 400, although another person can actuate the nasal spray device 400 for delivery into another person.

The actuation, e.g., depressing, of the dispensing head 406 is configured to cause venting air to enter the drug holder 402, as shown by arrow 416 in FIG. 4. The air entering the drug holder 402 displaces drug in the drug holder through a tube 418 and then into a metering chamber 420, which displaces drug proximally through a cannula 422, through the spray chamber 412, and then out of the opening 404. In response to release of the dispensing head 406, e.g., a user stops pushing downward on the dispensing head 406, a bias spring 426 causes the dispensing head 406 to return to its default, resting position to position the dispensing head 406 relative to the drug holder 402 for a subsequent actuation and drug delivery.

While the foregoing description relates to one particular example of a nasal spray device, this example is purely illustrative. The description should not be seen as limited only to such a nasal spray device. A person skilled in the art understands that the nasal spray device 400 can include different features in different embodiments depending upon various requirements. For example, the nasal spray device 400 can lack the depth guide 410 and/or may include any one or more of a device indicator, a sensor, a communications interface, a processor, a memory, and a power supply.

The nasal spray devices of the present disclosure can be used to administer any of a variety of drugs, such as any of ketamine (e.g., Ketalar®), esketamine (e.g., Spravato®, Ketanest®, and Ketanest-S®), naloxone (e.g., Narcan®), and sumatriptan (e.g., Imitrex®).

### Drug Administration Device

As will be appreciated from the foregoing, various components of drug delivery devices are common to all such devices. These components form the essential components of a universal drug administration device. A drug administration device delivers a drug to a patient, where the drug is provided in a defined dosage form within the drug administration device.

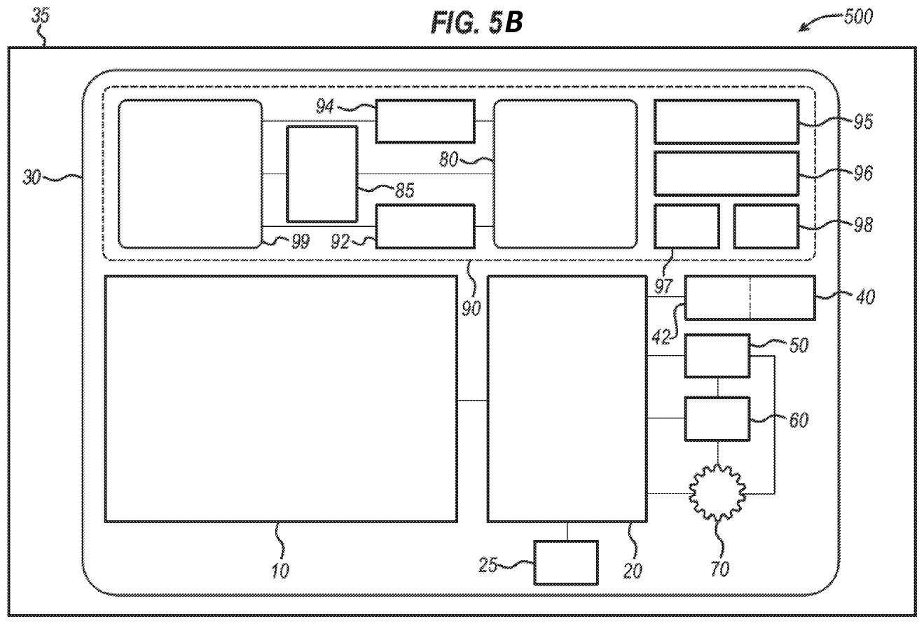

FIG. 5 is a generalized schematic view of such a universal drug administration device 501, and FIG. 5B is an exemplary embodiment of such a universal drug administration device 500. Examples of the universal drug administration device 500 include injection devices (e.g., autoinjectors, jet injectors, and infusion pumps), nasal spray devices, and inhalers.

As shown in FIG. 5A, drug administration device 501 includes in general form the features of a drug holder 10 and a dispensing mechanism 20. The drug holder 10 holds a drug in a dosage form to be administered. The dispensing mechanism 20 is configured to release the dosage form from the drug holder 10 so that the drug can be administered to a patient.

FIG. 5B shows a further universal drug administration device 500 which includes a number of additional features. A person skilled in the art understands that these additional features are optional for different embodiments, and can be utilized in a variety of different combinations such that the additional features may be present or may be omitted from a given embodiment of a particular drug administration device, depending upon requirements, such as the type of drug, dosage form of the drug, medical indication being treated with the drug, safety requirements, whether the device is powered, whether the device is portable, whether the device is used for self-administration, and many other requirements which will be appreciated by a person skilled in the art. Similar to the universal device of FIG. 5A, the drug administration device 500 comprises a housing 30 which accommodates the drug holder 10 and dispensing mechanism 20.

The device 500 is provided with a triggering mechanism 50 for initiating the release of the drug from the drug holder 10 by the dispensing mechanism 20. The device 500 includes the feature of a metering/dosing mechanism 70 which measures out a set dose to be released from the drug holder 10 via the dispensing mechanism 20. In this manner, the drug administration device 500 can provide a known dose of determined size. The device 500 comprises a dosage selector 60 which enables a user to set the dose volume of drug to be measured out by the metering mechanism 50. The dose volume can be set to one specific value of a plurality of predefined discrete dose volumes, or any value of predefined dose volume within a range of dose volumes.

The device 500 can comprise a device operation prevention mechanism 40 or 25 which when in a locked state will prevent and/or stop the dispensing mechanism 20 from releasing the drug out of the drug holder 10, and when in an unlocked state will permit the dispensing mechanism 20 to release the drug dosage from out of the drug holder 10. This can prevent accidental administration of the drug, for example to prevent dosing at an incorrect time, or for preventing inadvertent actuation. The device 500 also includes a dispensing mechanism protection mechanism 42 which prevents access to at least a part of the dispensing mechanism 20, for example for safety reasons. Device operation prevention mechanism 40 and dispensing mechanism protection mechanism 42 may be the same component.

The device 500 can include a device indicator 85 which is configured to present information about the status of the drug administration device and/or the drug contained therein. The device indicator 85 may be a visual indicator, such as a display screen, or an audio indicator. The device 500 includes a user interface 80 which can be configured to present a user of the device 500 with information about the device 500 and/or to enable the user to control the device 500. The device 500 includes a device sensor 92 which is configured to sense information relating to the drug administration device and/or the drug contained therein, for example dosage form and device parameters. As an example, in embodiments which include a metering mechanism 70 and a dosage selector 60, the embodiment may further include one or more device sensors 92 configured to sense one or more of: the dose selected by a user using dosage selector 60, the dose metered by the metering mechanism 70 and the dose dispensed by the dispensing mechanism 20. Similarly, an environment sensor 94 is provided which is configured to sense information relating to the environment in which the device 500 is present, such as the temperature of the environment, the humidity of the environment, location, and time. There may be a dedicated location sensor 98 which is configured to determine the geographical location of the device 500, e.g., via satellite position determination, such as GPS. The device 500 also includes a communications interface 99 which can communicate externally data which has been acquired from the various sensors about the device and/or drug.

If required, the device 500 comprises a power supply 95 for delivering electrical power to one or more electrical components of the device 500. The power supply 95 can be a source of power which is integral to device 500 and/or a mechanism for connecting device 500 to an external source of power. The drug administration device 500 also includes a device computer system 90 including processor 96 and memory 97 powered by the power supply 95 and in communication with each other, and optionally with other electrical and control components of the device 500, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

In some embodiments, the drug administration device 500 is enclosed in packaging 35. The packaging 35 may further include a combination of a processor 96, memory 97, user interface 80, device indicator 85, device sensor 92, location sensor 98 and/or environment sensors 94 as described herein, and these may be located externally on the housing of the device 500.

A person skilled in the art will appreciate that the universal drug administration device 500 comprising the drug holder 10 and dispensing mechanism 20 can be provided with a variety of the optional features described above, in a number of different combinations. Moreover, the drug administration device 500 can include more than one drug holder 10, optionally with more than one dispensing mechanism 20, such that each drug holder has its own associated dispensing mechanism 20.

### Drug Dosage Forms

Conventionally, drug administration devices utilize a liquid dosage form. It will be appreciated, however that other dosage forms are available.

One such common dosage form is a tablet. The tablet may be formed from a combination of the drug and an excipient that are compressed together. Other dosage forms are pastes, creams, powders, ear drops, and eye drops.

Further examples of drug dosage forms include dermal patches, drug eluting stents and intrauterine devices. In these examples, the body of the device comprises the drug and may be configured to allow the release of the drug under certain circumstances. For example, a dermal patch may comprise a polymeric composition containing the drug. The polymeric composition allows the drug to diffuse out of the polymeric composition and into the skin of the patient. Drug eluting stents and intrauterine devices can operate in an analogous manner. In this way, the patches, stents and intrauterine devices may themselves be considered drug holders with an associated dispensing mechanism.

Any of these dosage forms can be configured to have the drug release initiated by certain conditions. This can allow the drug to be released at a desired time or location after the dosage form has been introduced into the patient. In particular, the drug release may be initiated by an external stimulus. Moreover, these dosage forms can be contained prior to administration in a housing, which may be in the form of packaging. This housing may contain some of the optional features described above which are utilized with the universal drug administration device 500.

The drug administered by the drug administration devices of the present disclosure can be any substance that causes a change in an organism's physiology or psychology when consumed. Examples of drugs that the drug administration devices of the present disclosure can administer include 5-alpha-reductase inhibitors, 5-aminosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, aminoglycosides, aminopenicillins, aminosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, decongestants, dermatological agents, diagnostic radiopharmaceuticals, diarylquinolines, dibenzazepine anticonvulsants, digestive enzymes, dipeptidyl peptidase 4 inhibitors, diuretics, dopaminergic antiparkinsonism agents, drugs used in alcohol dependence, echinocandins, EGFR inhibitors, estrogen receptor antagonists, estrogens, expectorants, factor Xa inhibitors, fatty acid derivative anticonvulsants, fibric acid derivatives, first generation cephalosporins, fourth generation cephalosporins, functional bowel disorder agents, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, ketolides, laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, macrolide derivatives, macrolides, magnetic resonance imaging contrast media, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous antihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, parathyroid hormone and analogs, PARP inhibitors, PCSK9 inhibitors, penicillinase resistant penicillins, penicillins, peripheral opioid receptor antagonists, peripheral opioid receptor mixed agonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, quinolones, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, Streptomyces derivatives, succinimide anticonvulsants, sulfonamides, sulfonylureas, synthetic ovulation stimulants, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacient, topical steroids, topical steroids with anti-infectives, transthyretin stabilizers, triazine anticonvulsants, tricyclic antidepressants, trifunctional monoclonal antibodies, ultrasound contrast media, upper respiratory combinations, urea anticonvulsants, urea cycle disorder agents, urinary anti-infectives, urinary antispasmodics, urinary pH modifiers, uterotonic agents, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, or VMAT2 inhibitors. The drug administration devices of the present disclosure may administer a drug selected from epinephrine, Rebif, Enbrel, Aranesp, atropine, pralidoxime chloride, diazepam, insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, zoledronic acid, mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), Spravato® (esketamine), ketamine, and Invega Trinza® (paliperidone palmitate).

### Drug Housing

As described above, a dosage form can be provided in a holder that is appropriate for the particular dosage form being utilized. For example, a drug in a liquid dosage form can be held prior to administration within a holder in the form of a vial with a stopper, or a syringe with a plunger. A drug in solid or powder dosage form, e.g., as tablets, may be contained in a housing which is arranged to hold the tablets securely prior to administration.

The housing may comprise one or a plurality of drug holders, where each holder contains a dosage form, e.g., the drug can be in a tablet dosage form and the housing can be in the form of a blister pack, where a tablet is held within each of a plurality of holders. The holders being in the form of recesses in the blister pack.

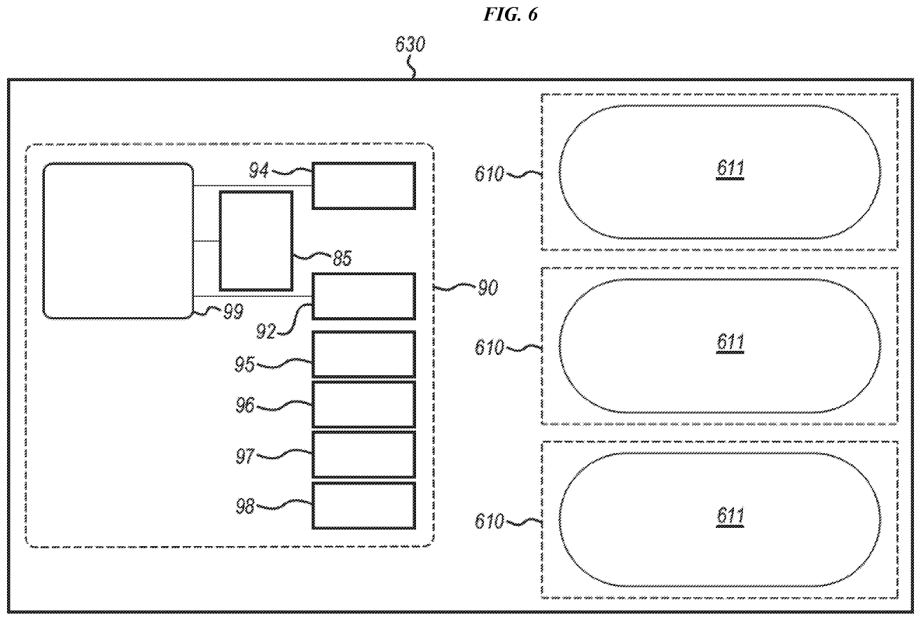

FIG. 6 depicts a housing 630 that comprises a plurality of drug holders 610 that each contain a dosage form 611. The housing 630 may have at least one environment sensor 94, which is configured to sense information relating to the environment in which the housing 630 is present, such as the temperature of the environment, time or location. The housing 630 may include at least one device sensor 92, which is configured to sense information relating to the drug of the dosage form 611 contained within the holder 610. There may be a dedicated location sensor 98 which is configured to determine the geographical location of the housing 630, e.g., via satellite position determination, such as GPS.

The housing 630 may include an indicator 85 which is configured to present information about the status of the drug of the dosage form 611 contained within the holder 610 to a user of the drug housing. The housing 630 may also include a communications interface 99 which can communicate information externally via a wired or wireless transfer of data pertaining to the drug housing 630, environment, time or location and/or the drug itself.

If required, the housing 630 may comprise a power supply 95 for delivering electrical power to one or more electrical components of the housing 630. The power supply 95 can be a source of power which is integral to housing 630 and/or a mechanism for connecting the housing 630 to an external source of power. The housing 630 may also include a device computer system 90 including processor 96 and memory 97 powered by the power supply 95 and in communication with each other, and optionally with other electrical and control components of the housing 630, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

The housing 630 can be in the form of packaging. Alternatively, additional packaging may be present to contain and surround the housing 630.

The holder 610 or the additional packaging may themselves comprise one or more of the device sensor 92, the environment sensor 94, the indicator 85, the communications interface 99, the power supply 95, location sensor 98, and device computer system including the processor 96 and the memory 85, as described above.

### Electronic Communication

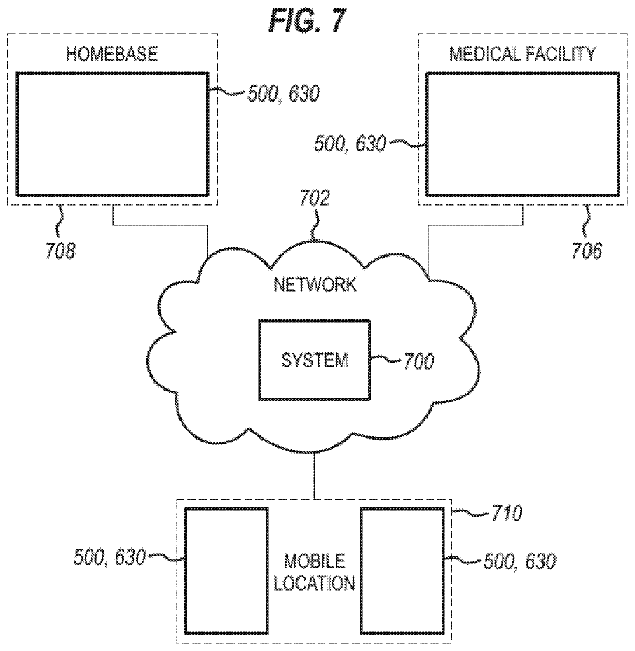

As mentioned above, communications interface 99 may be associated with the drug administration device 500 or drug housing 630, by being included within or on the housing 30, 630, or alternatively within or on the packaging 35. Such a communications interface 99 can be configured to communicate with a remote computer system, such as central computer system 700 shown in FIG. 7. As shown in FIG. 7, the communications interface 99 associated with drug administration device 500 or housing 630 is configured to communicate with a central computer system 700 through a communications network 702 from any number of locations such as a medical facility 706, e.g., a hospital or other medical care center, a home base 708 (e.g., a patient's home or office or a care taker's home or office), or a mobile location 710. The communications interface 99 can be configured to access the system 700 through a wired and/or wireless connection to the network 702. In an exemplary embodiment, the communications interface 99 of FIG. 6 is configured to access the system 700 wirelessly, e.g., through Wi-Fi connection(s), which can facilitate accessibility of the system 700 from almost any location in the world.

A person skilled in the art will appreciate that the system 700 can include security features such that the aspects of the system 700 available to any particular user can be determined based on, e.g., the identity of the user and/or the location from which the user is accessing the system. To that end, each user can have a unique username, password, biometric data, and/or other security credentials to facilitate access to the system 700. The received security parameter information can be checked against a database of authorized users to determine whether the user is authorized and to what extent the user is permitted to interact with the system, view information stored in the system, and so forth.

### Computer System

As discussed herein, one or more aspects or features of the subject matter described herein, for example components of the central computer system 700, processor 96, power supply 95, memory 97, communications interface 99, user interface 80, device indicators 85, device sensors 92, environment sensors 94 and location sensors 98, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computer system may include clients and servers. A client and server are generally remote from each other and typically interact through a communications network, e.g., the Internet, a wireless wide area network, a local area network, a wide area network, or a wired network. The relationship of client and server arises by virtue of computer programs running on the respective computers and having a client-server relationship to each other.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein, for example user interface 80 (which can be integrated or separate to the administration device 500 or housing 630), can be implemented on a computer having a display screen, such as for example a cathode ray tube (CRT) or a liquid crystal display (LCD) or a light emitting diode (LED) monitor for displaying information to the user. The display screen can allow input thereto directly (e.g., as a touch screen) or indirectly (e.g., via an input device such as a keypad or voice recognition hardware and software). Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. As described above, this feedback may be provided via one or more device indicators 85 in addition to the user interface 80. The device indicators 85 can interact with one or more of device sensor(s) 92, environment sensor(s) 94 and/or location sensor(s) 98 in order to provide this feedback, or to receive input from the user.

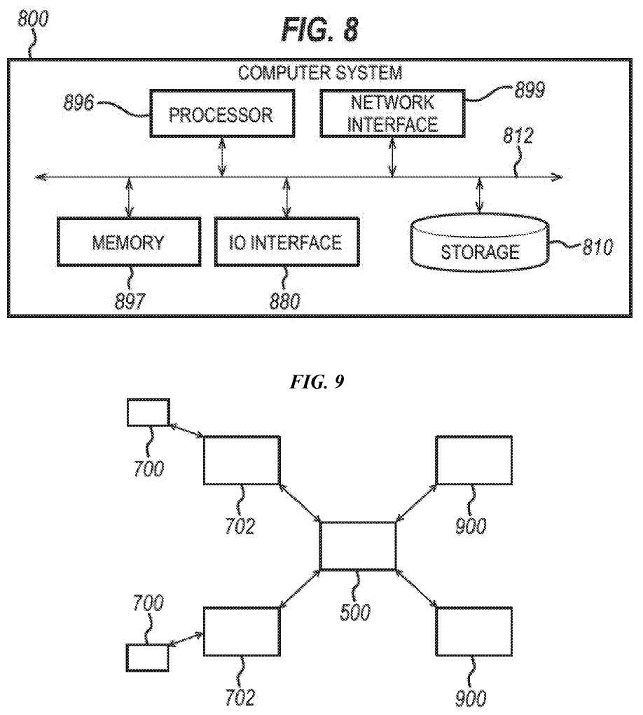

FIG. 8 illustrates one exemplary embodiment of the computer system 700, depicted as computer system 800. The computer system includes one or more processors 896 configured to control the operation of the computer system 800. The processor(s) 896 can include any type of microprocessor or central processing unit (CPU), including programmable general-purpose or special-purpose microprocessors and/or any one of a variety of proprietary or commercially available single or multi-processor systems. The computer system 800 also includes one or more memories 897 configured to provide temporary storage for code to be executed by the processor(s) 896 or for data acquired from one or more users, storage devices, and/or databases. The memory 897 can include read-only memory (ROM), flash memory, one or more varieties of random access memory (RAM) (e.g., static RAM (SRAM), dynamic RAM (DRAM), or synchronous DRAM (SDRAM)), and/or a combination of memory technologies.

The various elements of the computer system are coupled to a bus system 812. The illustrated bus system 812 is an abstraction that represents any one or more separate physical busses, communication lines/interfaces, and/or multi-drop or point-to-point connections, connected by appropriate bridges, adapters, and/or controllers. The computer system 800 also includes one or more network interface(s) 899 (also referred to herein as a communications interface), one or more input/output (TO) interface(s) 880, and one or more storage device(s) 810.

The communications interface(s) 899 are configured to enable the computer system to communicate with remote devices, e.g., other computer systems and/or devices 500 or housings 630, over a network, and can be, for example, remote desktop connection interfaces, Ethernet adapters, and/or other local area network (LAN) adapters. The IO interface(s) 880 include one or more interface components to connect the computer system 800 with other electronic equipment. For example, the IO interface(s) 880 can include high speed data ports, such as universal serial bus (USB) ports, 1394 ports, Wi-Fi, Bluetooth, etc. Additionally, the computer system can be accessible to a human user, and thus the IO interface(s) 880 can include displays, speakers, keyboards, pointing devices, and/or various other video, audio, or alphanumeric interfaces. The storage device(s) 810 include any conventional medium for storing data in a non-volatile and/or non-transient manner. The storage device(s) 810 are thus configured to hold data and/or instructions in a persistent state in which the value(s) are retained despite interruption of power to the computer system. The storage device(s) 810 can include one or more hard disk drives, flash drives, USB drives, optical drives, various media cards, diskettes, compact discs, and/or any combination thereof and can be directly connected to the computer system or remotely connected thereto, such as over a network. In an exemplary embodiment, the storage device(s) 810 include a tangible or non-transitory computer readable medium configured to store data, e.g., a hard disk drive, a flash drive, a USB drive, an optical drive, a media card, a diskette, or a compact disc.

The elements illustrated in FIG. 8 can be some or all of the elements of a single physical machine. In addition, not all of the illustrated elements need to be located on or in the same physical machine.

The computer system 800 can include a web browser for retrieving web pages or other markup language streams, presenting those pages and/or streams (visually, aurally, or otherwise), executing scripts, controls and other code on those pages/streams, accepting user input with respect to those pages/streams (e.g., for purposes of completing input fields), issuing HyperText Transfer Protocol (HTTP) requests with respect to those pages/streams or otherwise (e.g., for submitting to a server information from the completed input fields), and so forth. The web pages or other markup language can be in HyperText Markup Language (HTML) or other conventional forms, including embedded Extensible Markup Language (XML), scripts, controls, and so forth. The computer system 800 can also include a web server for generating and/or delivering the web pages to client computer systems.

As shown in FIG. 7, the computer system 800 of FIG. 8 as described above may form the components of the central computer system 700 which is in communication with one or more of the device computer systems 90 of the one or more individual drug administration devices 500 or housings 630. Data, such as operational data of the devices 500 or housings 630, medical data acquired of patients by such devices 500 or housings 630 can be exchanged between the central and device computer systems 700, 90.

As mentioned the computer system 800 as described above may also form the components of a device computer system 90 which is integrated into or in close proximity to the drug administration device 500 or housing 630. In this regard, the one or more processors 896 correspond to the processor 96, the network interface 799 corresponds to the communications interface 99, the IO interface 880 corresponds to the user interface 80, and the memory 897 corresponds to the memory 97. Moreover, the additional storage 810 may also be present in device computer system 90.

In an exemplary embodiment, the computer system 800 can form the device computer system 90 as a single unit, e.g., contained within a single drug administration device housing 30, contained within a single package 35 for one or more drug administration devices 500, or a housing 630 that comprises a plurality of drug holders 610. The computer system 800 can form the central computer system 700 as a single unit, as a single server, or as a single tower.

The single unit can be modular such that various aspects thereof can be swapped in and out as needed for, e.g., upgrade, replacement, maintenance, etc., without interrupting functionality of any other aspects of the system. The single unit can thus also be scalable with the ability to be added to as additional modules and/or additional functionality of existing modules are desired and/or improved upon.

The computer system can also include any of a variety of other software and/or hardware components, including by way of example, operating systems and database management systems. Although an exemplary computer system is depicted and described herein, it will be appreciated that this is for sake of generality and convenience. In other embodiments, the computer system may differ in architecture and operation from that shown and described here. For example, the memory 897 and storage device 810 can be integrated together or the communications interface 899 can be omitted if communication with another computer system is not necessary.

### Implementations

Communicating with External Systems

FIG. 9 illustrates an embodiment of a drug administration system including a drug administration device 500 (FIG. 5B), a plurality of central computer systems 700 (FIG. 7), and a plurality of external devices 900. The communications interface 99 of the drug administration device 500 is configured to communicate with the computer systems 700 through communications networks 702 (FIG. 7) and with the external devices 900. The communication with the external devices 900 can be through the illustrated communications networks 702 and/or other communications network(s) 702. Although two communications networks 702 are shown in FIG. 9, the two communications networks 702 can instead be a single communications network 702 or be more than two communications networks 702, as will be appreciated by a person skilled in the art and as discussed above. Additionally, although two external devices 900 and two computer systems 700 are shown in FIG. 9, another number of each of the external devices 900 and the computer systems 700 can be present in the drug administration system, e.g., no external devices 900 and at least one computer system 700, no computer systems 700 and at least one external device 900, one external device 900 and one or more computer systems 700, one computer system 700 and one or more external devices, or a plurality of computer systems 700 and a plurality of external devices 900.

The drug administration device 500 is characterized by a number of parameters of the drug administration device 500. The parameters of the drug administration device 500 (also referred to herein as “device parameters”) can relate to any characteristics of the drug administration device 500. In general, the device parameters describe an operational state of the drug administration device 500 and a drug held within the drug administration device 500 (e.g., within the drug holder 10) for delivery from the drug administration device 500 to a patient. Examples of device parameters include an administration history of the drug, times of previous doses of the drug delivered from the device 500, amounts of previous doses of the drug delivered from the device 500, an amount of the drug remaining in the drug administration device 500 (e.g., remaining within the drug holder 10) such as volume, mass, and/or molar amount of the drug remaining in the drug administration device 500, a chemical/pharmaceutical composition of the drug, and a power consumption of the drug administration device 500 (e.g., a duration of life remaining in the device's power supply 95) at a present rate of power consumption. In other words, the device parameters are variables each describing a feature of the drug administration device 500. The device parameters can help in assessing performance of the drug administration device 500 and thus may assist in optimizing operation of the drug administration device 500.

As discussed above, the device sensor 92 of the drug administration device 500 is configured to obtain sensor data relating to the drug administration device 500 and/or the drug contained therein. For example, the device sensor 92 of the drug administration device 500 can be configured to sense device parameters including one or more of orientation of the drug administration device 500 (e.g., with the device sensor 92 being a gyro, an accelerometer, a tilt/angle switch (mercury free), a position sensor, etc.) and a temperature of the drug in the drug administration device 500 (e.g., with the device sensor 92 being a temperature sensor such as a thermistor, a thermocoupler, a thermistor, etc.).

The parameters of the drug administration device 500 can be recorded as drug administration data within the drug administration device 500, e.g., recorded in the memory 97 of the drug administration device 500. The drug administration data is compiled in a first data format. That is, the drug administration data is stored according to a predefined organizational regime. In other words, the drug administration data is stored in a particular software file type.

The device sensor 92 of the drug administration device 500 is configured to sense information relating to the drug administration device 500 and/or the drug contained therein, as discussed above, and the sensed data can be included as one or more of the device parameters of the drug administration device 500. The computer systems 700 are each configured as discussed above and each include a system with which the drug administration device 500 can establish communications through the respective networks 702. Some computer systems 700 are specifically designed for communication with the drug administration device 500, whereas other computer systems 700 merely allow other devices, such as the drug administration device 500, to communicate with them. The networks 702 are configured as discussed above and are each be any network compatible for communication with its respective computer system 700 and with the drug administration device 500.

The external devices 900 each include any device external to and with which the drug administration device 500 can establish communications. Some external devices 900 are specifically designed for communication with the drug administration device 500, whereas other external devices 900 merely allow other devices, such as the drug administration device 500, to communicate with them.

There are numerous examples of external devices 900. In general, each of the external devices 900 is a device separate from the drug administration device 500. The external devices 900 can each be configured to record or influence a physiological parameter of a user (also referred to herein as a “user parameter” or a “user physiological parameter”), such as by using a sensor. A user parameter relates to a condition (characteristic) of a user of the drug administration device 500, e.g., the patient. The user parameter can relate to a physiological condition of the user, such as the user's heart rate, blood glucose level, blood pressure, body temperature, heart rhythm, heart rate variability, fatigue level, respiration level, concentration of a drug in the blood, etc. The user parameter can relate to an environmental condition of the user, e.g., a condition of a local environment of the user, such as location, temperature, humidity, pollution level, pollen level, UV level, etc. A user stress testing device (e.g., a standalone stress testing device or a device that includes stress testing capabilities as a feature and/or app thereof) and a user exercise device (with electronic functionality) are each examples of external devices 900 and are each configured to record a physiological parameter, such as heart rate, and influence that parameter by facilitating physical exertion of the user. A fitness wearable is another example of an external device 900 and is configured to record a user parameter, such as the user's location and/or heart rate. A heart rate monitor is another example of an external device 900 and is configured to monitor a user's heart rate. A blood glucose monitor is another example of an external device 900 and is configured to monitor a user's blood glucose level. A continuous positive airway pressure (CPAP) machine is another example of an external device 900 and is configured to control a user's breathing and measure a resistance in a user's breathing. A respiration meter is another example of an external device 900 and is configured to measure a resistance in a user's breathing. A fatigue meter is another example of an external device 900 and is configured to measure exertion level. A motion sensor is another example of an external device and is configured to measure motion. A mass meter is another example of an external device and is configured to measure mass. Some external devices are configured to put a user into a certain physiological condition when the external device 900 is in use, which enables baseline/reference measurements of various user and drug administration parameters to be determined. Examples of such an external device 900 includes a running machine and a stress testing device. A pollen sensor is another example of an external device 900 and is configured to measure pollen level. A temperature sensor (a temperature sensor such as a thermistor, a thermocoupler, a thermistor, etc.) is another example of an external device 900 and is configured to measure temperature. A humidity sensor (e.g., a thermistor, a humistor, a hygrometer, etc.) is another example of an external device 900 and is configured to measure humidity. A UV sensor is another example of an external device 900 and is configured to measure air quality (pollution level). Measures of air quality include measures of nitrogen dioxide, ground-level ozone, carbon monoxide, and particulates. Other examples of user parameters include location (e.g., with the external device including a location sensor similar to the location sensor 98 discussed above), blood oxygen level (e.g., with the external device 900 being a blood oxygen sensor), blood pressure (e.g., with the external device 900 being a blood pressure monitor, etc.), tissue thickness (e.g., with the external device 900 being an impedance sensor, etc.), tissue density (e.g., with the external device 900 being an impedance sensor, etc.), speed or velocity (e.g., with the external device 900 being a speedometer, etc.), travel distance (e.g., with the external device 900 being a odometer, pedometer, etc.).

The computer systems 700 and the external devices 900 are each configured to store or measure ancillary data. Ancillary data refers to any data that is stored or measured by the computer systems 700 or the external devices 900, e.g., ancillary data is data that originates from a source external to the drug administration device 500. The ancillary data includes one or more data streams. Each data stream relates to a different parameter. For example, there can be a data stream relating to a user parameter such as heart rate, another data stream relating to an external device parameter such as a battery life of the external device 900, and another data stream relating to a computer system parameter such as a battery life of the computer system 700. Thus, some ancillary data can relate to one or more user parameters and some ancillary data can relate to parameters that are not user parameters, such as external device parameters (e.g., parameters related to characteristics of the external devices 900 such as an operational status of the external devices 900, battery life of the external devices 900, etc.) and computer system parameters (e.g., parameters related to characteristics of the computer systems 700 such as an operational status of the computer systems 700, battery life of the computer systems 700, etc.). The ancillary data can include a combination of different types of parameters, e.g., different types of user parameters, combinations of different physiological conditions of the user and/or different environmental conditions of the user, different types of external device parameters, and different types of computer system parameters.

Some ancillary data is relevant to drug administration or the user, whereas other ancillary data is not relevant to drug administration or the user. Relevant information is any information that may have an effect on determining an optimal future dosage to be administered to the patient from the drug administration device 500. Thus, some data streams of ancillary data can relate to parameters that are relevant, whereas other data streams of ancillary data can relate to irrelevant parameters. Accordingly, user parameters are relevant, since these have a relationship with drug administration from the drug administration device 500 to the user, whereas data relating to color of a housing or a battery life of the external device 900 or computer system 700 is not relevant since these do not have a relationship with drug administration from the drug administration device 500 to the user. However, depending on what drug is being administered, only some user parameters may be relevant. For example, temperature may not be relevant if the drug in the drug administration device 500 is not affected (e.g., remains potent, remains at a deliverable viscosity, can be comfortably delivered to the patient, etc.) by swings in temperature. For another example, blood glucose level may not be relevant if the drug in the drug administration device 500 is not related to treatment of a blood glucose disorder. For another example, pollution level and a user's breathing may not be relevant if the drug administration device 500 is not an inhaler (e.g., the inhaler 300 of FIG. 3).

An example of relevant ancillary data includes the location of the user. The location of the user can be used, e.g., by a processor, to determine (for example, by matching a GPS location to a weather map) an air temperature and/or a pollen count at the user's location (or the user's approximate location depending on the sensitivity of the location sensor being used and/or on available weather data, e.g., data being specific to a city in which the user is located but not being to a specific region within the city at which the user is located, data being used from a weather station not precisely at but near the user's current location, etc.). Parameters of air temperature and pollen count may impact on health of the user. For example, an asthmatic user may be at increased risk of an asthma attack in locations with a high pollen count. Hence, a data stream containing pollen level information can be used, e.g., by a processor, to determine that an increased dose of an asthma attack prevention drug should be administered from the drug administration device 500 in the case of the drug administration device 500 being an inhaler (e.g., the inhaler 300 of FIG. 3). The increased dose can be determined, for example, using a lookup table stored in a memory (e.g., the memory 97 of the drug administration device 500) that a processor (e.g., the processor 96 of the drug administration device 500) is configured to access to look up a drug dose corresponding to the measured pollen level.

Another example of relevant ancillary data includes the environmental condition of the user. Environmental conditions can relate to an amount of exercise a user is currently undertaking. For example, if a diabetic user is using a running machine, a speed at which the user is running, as measured by the running machine, can be used, e.g., by a processor, to predict a rate of glucose consumption by the user and, hence, be used to predict when and how much insulin should be delivered in the case of the drug administration device 500 containing insulin to be delivered to the user. Similarly, a measurement of pollen count (pollen level) can be used, e.g., by a processor, to adjust a drug dosage, as described above.

A further example of relevant ancillary data includes the physiological condition of the user. Physiological condition data can be used, e.g., by a processor, to assess when the drug should be delivered to the user from the drug administration device 500 and to determine how the user is reacting to the current treatment. For example, a drop in blood glucose level may require an adjustment of the drug dosage. In another example, physiological condition(s) may indicate that the user is becoming immune to the drug or is allergic to the drug and that, therefore, corresponding adjustments should be made to the drug dosage or that use of the drug should be ceased with another drug being prescribed to the user that the user is not immune or allergic to.

Another example of relevant ancillary data includes the psychological condition of the user. In general, psychological conditions relate to a mental state of a user. Psychological condition data can be gathered in a variety of ways, such as via patient answers to questions in a questionnaire that are stored at the external device 900, through user input to one or more questions presented via a drug administration device's user interface such as answers to a psychological stress test such as the Kessler Psychological Distress Scale (K10) or any of a variety of other indices and scales, health care provider assessment notes regarding the patient that are stored at the external device, etc. Psychological condition data can be used, e.g., by a processor, to assess when the drug should be delivered to the user from the drug administration device 500 and to determine how the user is reacting to the current treatment. For example, a trend in improving mental state may be indicative of effective drug treatment for depression such that drug dosage and/or drug dosing frequency may be reduced. For another example, a trend in declining mental state or a static trend in mental state may be indicative that drug dosage and/or drug dosing frequency should be increased for a drug treating depression.

The external devices 900 are each a computer system similar to that discussed above regarding the computer system 800 of FIG. 9. The external devices 900 each include at least a communications interface configured to communicate with the drug administration device 500, a sensor configured to measure data, and a processor configured to control the sensor and the communications interface. The communications interface of the external device 900 is configured to communicate ancillary data (e.g., data measured by the sensor) to the drug administration device 500, e.g., to the communication interface 99 of the drug administration device 500. The external device 900 can include other components as discussed above, such as a memory configured to store data measured by the sensor permanently or temporarily.

The processor of the external device 900 is configured to control the sensor of the external device 900 to measure a user parameter, such as a physiological or environmental condition, continuously, at regular intervals, or when requested by the user or the drug administration device 500. Continuous measurement means that the sensor records a constant stream of samples of the user parameter. Measurement at regular intervals means that the sensor records one or more samples of the user parameter at predefined temporal separations. Requests by the user of drug administration device 500 can be requests to measure the user parameter continuously, or at regular intervals, or may be a one-off request to measure a single sample. The requests can be made, for example, by the user providing an input to the user interface 80 of the drug administration device 500 and the drug administration device's communications interface 99 communicating the input to the communications interface of the external device 900. For another example, the request can be made by the user providing an input to a user interface of the external device 900.

The computer systems 700 can each be configured to store relevant ancillary data in a memory thereof, for example, the computer system 700 can be configured to store weather data thereby enabling environmental parameters related to weather, such as temperature and pollen count, at or approximately at the patient's location to be determined. The ancillary data can be stored at the central computer systems 700 in communication with their respective networks 702. The networks 702 are configured to facilitate transmission of the ancillary data to the drug administration device 500, as discussed above.

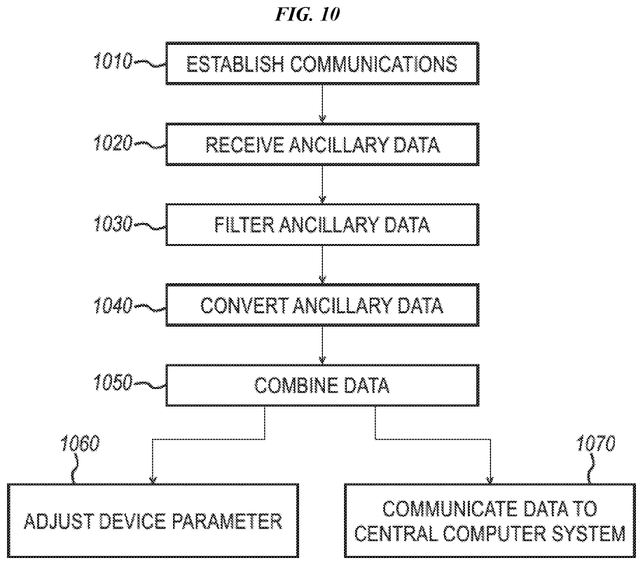

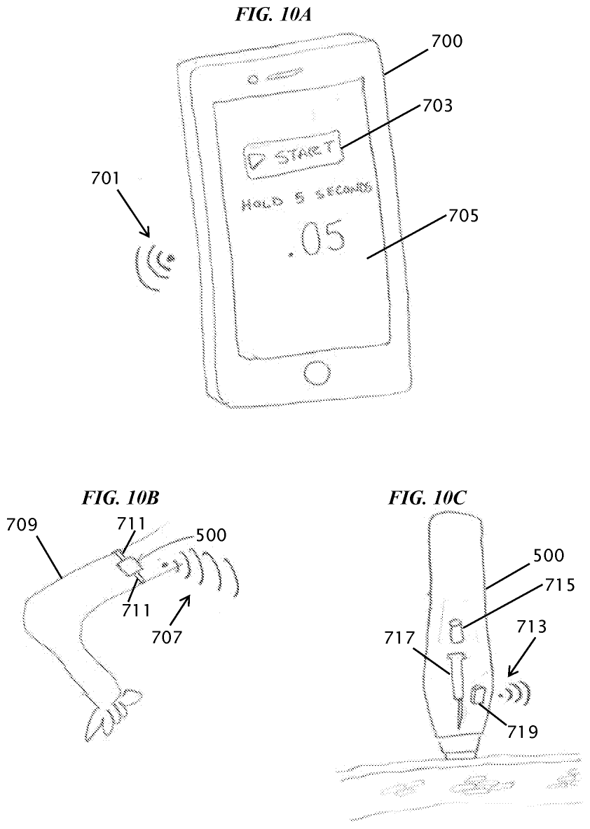

FIG. 10 illustrates an embodiment of using the drug administration system of FIG. 9. The drug administration device 500 establishes communications 1010 with the computer systems 700 and the external devices 900. The general principle is for the drug administration device 500 to gather data relevant to the user from the computer systems 700 and the external devices 900 and, hence, relevant to the drug administration. Therefore, the drug administration device 500 is designed to communicate with any surrounding computer systems 700 and external devices 900 within communication range of the drug administration device 500 in order to obtain this information.

In an exemplary embodiment, the communications interface 99 of the drug administration device 500 scans for computer systems 700 and external devices 900. The scanning allows the drug administration device 500 to discover or identify computer systems 700 and external devices 900 within communication range of the drug administration device 500 and external devices 900 that are available for the establishing of communications. For example, the communications interface 99 can search for computer systems 700 and external devices 900 using a wireless communications protocol. Examples of wireless communication protocols include WiFi, Ethernet, Bluetooth, Zigbee, radio frequency identification (RFID), near field communication (NFC), Li-Fi, Infrared, Bluetooth Low Energy (BLE), Z-Wave, cellular, optical, mobile phone networks, and other internet connections. Wireless communication removes the need for the drug administration device 500 to be physically connected to the networks 702 or external devices 900 and may thereby increase flexibility of utilizing the device 500. The communications interface 99 can search automatically (without user input) for all available computer systems 700 and external devices 900. Having identified available computer systems 700 and external devices 900, the communications interface 99 attempts to connect to the available computer systems 700 and external devices 900, that is, the communications interface 99 attempts to establish a communications link. Establishment of a communications link is either automatic or requires a user input to authorize connection.

Establishing communications 1010 can include using a key-based security system, such as a public key/private key cryptographic system, to allow for data encryption and decryption. Public and private keys can be stored in a memory and can be generated using cryptographic algorithms. Keys can be used to encrypt data for transmission and to decrypt encrypted data received from a different computing device. In such systems, a public key associated with the intended receiver of the data can be utilized to encrypt data, however, only the recipient's private key can be used to decrypt the encrypted data. In at least some embodiments, a cryptographic system such as a public key infrastructure (PM), in which one or more third parties, known as “certificate authorities,” can be used to certify ownership of the public and private key pairs. Examples of key-based security systems include the Diffie-Hellman key exchange protocol, the Digital Signature Standard (DSS) protocol, password-authenticated key agreement protocols, the Rivest-Shamir-Adelman (RSA) encryption algorithm, the Cramer-Shoup cryptosystem, and the YAK authenticated key agreement protocol. Any type of encryption (including WEP, WPA, and WPA2 encryption methods) can be used to encrypt transmitted data. Various digital certificate validation schemes and cryptographic protocols, including the Secure Sockets Layer protocol (SSL), the Transport Layer Security protocol (TLS), RSA, or any other public/private key protocols can be utilized in establishing the communication.

In an exemplary embodiment, the drug administration device 500 seeks to establish communications 1010 with as many computer systems 700 and external devices 900 as possible in order to gather the maximum amount of relevant data. In other words, the drug administration device 500 can discover and connect to all available computer systems 700 and external devices 900 that may be measuring data relevant to drug administration, such as a user parameter relevant to drug administration, which may enable accuracy of a determination of an optimum dose to be improved. The communications interface 99 either communicates with many computer systems 700 and external devices 900 simultaneously or continuously loops through a list of individual, consecutive connections with the available computer systems 700 and external devices 900, as will be appreciated by a person skilled in the art. Alternatively, the drug administration device 500 can be configured to seek to establish communications with a predetermined list of computer systems 700, networks 702, and external devices 900, which may security of the device 500 by only allowing the device 500 to communicate with trusted networks, systems, and devices.

Processes of establishing communication links between a drug administration device and a computer system or an external device are discussed further below.

The drug administration device 500 receives 1020 the ancillary data from the computer systems 700 and the external devices 900 using the communications interface 99. The ancillary data can be sent automatically by the computer systems 700 and the external devices 900, or the ancillary data can be sent upon request by the drug administration device 500 (e.g., by the drug administration device 500 sending an instruction to send ancillary data to the computer systems 700 and the external devices 900 with which communications have been established 1010). Different ones of the computer systems 700 and the external devices 900 can have automatic and requested communication.

As discussed above, the ancillary data contains relevant and irrelevant information. Filtering 1030 the ancillary data, e.g., using the processor 96 of the device 500, removes the irrelevant information leaving only the relevant information. Filtering thus allows retention of only information relevant to drug administration as discussed above, for example data relating to user parameters. Depending on the circumstances, for example, a type of drug being administered, only some of the user parameters may be relevant as discussed above. Any user parameters that are retained after the filtering 1030 are the user parameters that are considered to be of interest for subsequent processing in determining whether to adjust drug delivery from the device 500. For example, when only a heart rate of the user is of interest for the subsequent processing, then any other user parameters that are present in the ancillary data can be filtered out. As part of the filtering 1030, any other data that is considered irrelevant for the subsequent processing can also be removed, such as data that is associated with the original data format of the ancillary data or data relating to the operational status of the computer system 700 or external device 900 that transmitted the ancillary data to the device 500.

The filtering 1030 can be performed by the processor 96 in a variety of ways. In an exemplary embodiment, the processor 96 identifies the parameter to which each data stream of the ancillary data relates. For example, the processor 96 can determine that one data stream relates to heart rate whereas another data stream relates to battery life of one of the external devices 900. This determination can be achieved by checking a marker signal associated with each data stream that indicates that the data stream relates to a particular parameter. Having identified the parameter of each data stream, the processor 96 can determine which data streams are relevant. The memory 97 stores a list of relevant parameter types. The processor 96 can compare the parameters of the data streams to the list of relevant parameters stored in the memory 97 to determine which data streams are relevant. The irrelevant data can be deleted from the memory 97, e.g., the irrelevant data streams can be deleted, in order to save storage space in the memory 97 of the drug administration device 500. The relevant data streams are thereby extracted and are available for subsequent processing. Filtering 1030 the ancillary data may reduce an amount of data that must be subsequently processed by the processor, which reduces the processing time of the data and thereby optimizes operation of the drug administration device 500.

The processor 96 can also similarly filter the drug administration data. This filtering of the drug administration data removes any parameters of the drug administration device 500 that are irrelevant to the subsequent processing.

The ancillary data received at the drug administration device 500 from any one or more of the computer systems 700 and external devices 900 can be in a different data format to that of the drug administration data. The processor 96 determines whether received ancillary data is in a different data format or not. If the data format of the ancillary data is different, the processor 96 converts 1040 the data format of the ancillary data into that of the drug administration data, e.g., by executing an algorithm stored in the memory 97. As a result of the conversion 1040, both the ancillary data and the drug administration data are in the same format. This matching of formats may reduce complexity and increase speed of the further processing of the data, thereby optimizing operation of the drug administration device 500.

The conversion 1040 is shown in FIG. 10 as occurring after the filtering 1030, but the filtering 1030 can occur after the conversion 1040.

Following the filtering 1030 and conversion 1040, the drug administration data and the relevant ancillary data (e.g., that relating to user parameters) are combined 1050, e.g., combined into a single data packet. The combined data contains all available information relevant to drug administration as gathered from both the drug administration device 500 itself and the computer systems 700 and the external devices 900. The drug administration device 500 can thus act as a central information gathering point within easy access of the user and be able to combine data from a plurality of separate sources, including device parameters and user parameters, into a data packet with a uniform data format where the irrelevant data has been filtered out. Such a data packet may increase ease of handling the data due to smaller size of the data packet (as compared with a data packet including all of the relevant and irrelevant data) and/or only data that will be subsequently used by the processor 96 being present in the data packet since the data packet does not necessarily include all data received by the drug administration device 500 but rather only a selected part of the data received by the drug administration device 500. This collection of relevant data can be used to calculate an optimum dosage and improve the operation of the drug administration device 500, as discussed further below.

Optionally, the drug administration data and the ancillary data are combined in a synchronized manner. The drug administration data and the ancillary data can each include associated date/time values. This date/time stamping enables the processor 96 to determine that a data value in the drug administration data has the same associated time value as a data value in the ancillary data. These data values with matching date/time values can be combined such that they form a pair. This date/time value comparison and combination of matching date/time data values can be repeated for all data values so that the drug administration data and the ancillary data are combined in a synchronized manner. Synchronization enables events in one data type to be matched to events occurring at the same time in the other data types.

The processor 96 of the drug administration device 500 adjusts 1060 operational parameters based on analysis of the combined data, e.g., of the data packet. In other words, the processor 96 determines whether and by how much the operational parameters should be changed so that the drug administration device 500 operates in an optimal way based on the information in the combined data. The operational parameters are thus adjustable parameters. In general, an operational parameters is a parameter that relates to the administration of drug from the drug administration device 500. Examples of operational parameters include dosage amount (e.g., mass and/or volume of drug to be delivered in a subsequent dose), dosage frequency, and dosage time, e.g., the time at which the next dose should be administered. Examples of parameters of the drug administration device 500 that cannot be adjusted by the processor 96 include an amount of drug remaining in the drug administration device's drug holder 10 and a remaining amount of power in the power supply 95. These non-adjustable parameters can change over time and can be indirectly affected by the operational parameters.

The drug administration device 500 is configured to utilize a drug dosing scheme when the drug is delivered to the patient from the drug administration device 500. The combined data, e.g., the relevant drug administration data and the relevant ancillary data, indicates the operational parameters to be utilized by the drug administration device 500 when delivering the drug therefrom to the patient. In this way, the relevant drug administration data and the relevant ancillary data can be used as a basis for adjusting the drug dosing scheme. The drug dosing scheme is a collection of one or more operational parameters associated with the delivery of drug. Examples of operational parameters include at least one of drug delivery rate, drug delivery volume, drug delivery duration, drug delivery frequency, and timing of drug delivery. The drug dosing scheme is made up of one or more of such operational parameters.

In general, the dosing scheme is an algorithm stored in the memory 97 of the drug administration device 500 that is executable on board the drug administration device 500 by the processor 96. The algorithm is stored in the form of one or more sets of pluralities of data points defining and/or representing instructions, notifications, signals, etc. to control functions of the device 500 and administration of the drug from the device 500. As discussed herein, the drug administration data and the ancillary data received by the drug administration device 500, e.g., as pluralities of data points via the communications interface 99 thereof, can be used, e.g., by the processor 96, to adjust 1060 at least one operational parameter of the algorithm. The at least one operational parameter is among the algorithm's data points and are thus each able to be adjusted by changing one or more of the stored pluralities of data points of the algorithm.

The data gathered by the drug administration device 500, e.g., locally on board the device 500 and from the computer systems 700 and the external devices 900, represents input parameters that the drug administration device 500 is configured to use in executing a calculation (e.g., in execution of a calculation algorithm stored in the memory 97) to determine the correct value for each of the operational parameters in the algorithm for the drug dosing scheme. The calculation algorithm is stored in the memory 97 in the form of one or more sets of pluralities of data points. The calculation algorithm utilized by the drug administration device 500 can be already present in the memory 97 prior to combining 1050 the data.

After adjustment 1060 of the operational parameters has occurred (which may include no changes to any of the operational parameters or changes or changes to one or more of the operational parameters), subsequent execution of the algorithm administers another dose of the drug according to the algorithm, which reflects the current drug dosing scheme. Alternatively, each changed algorithm, e.g., each different drug dosing scheme, can be stored in the memory 97 with the processor 96 executing the most recent of the stored drug dosing schemes to cause drug delivery from the drug administration device 500. Storing each of the drug dosing schemes may require a larger memory 97 than if only one drug dosing scheme is stored at a time, but storing each of the drug dosing schemes simultaneously in the memory 97 facilitate analysis of drug delivery, patient compliance, and/or of the patient's treatment.

By allowing for changes in the drug dosing scheme over time, drug delivery over time can be managed for the patient to increase the beneficial results of the drug. Changing the operational parameters is automated to improve patient outcomes. Thus, the drug administration system can be configured to facilitate personalized medicine based on the patient to provide a smart system for drug delivery.

As mentioned above, the processor 96 of the drug administration device 500 adjusts 1060 the operational parameters based on analysis of the combined data, e.g., the data packet. The data packet, which contains relevant information relating to the current situation of the drug administration device 500 and the user, is used by the processor 96 to improve the operation of the drug administration device 500 by adjusting the drug dosing scheme. Utilizing the data packet, rather than unfiltered, separate data streams, may increase the efficiency associated with calculating changes to the operational parameters of the drug administration device 500 because the data packet contains only relevant information for the calculation.

The processor 96 calculates the changes to the operational parameters according to an adjustment algorithm stored in the memory 97 or according to a look-up table stored in the memory 97. The adjustment algorithm is one or more sets of pluralities of data points defining and/or representing instructions, notifications, signals, etc. similar to that discussed above with respect to the algorithm of the drug dosing scheme. For example, the adjustment algorithm may implement a mathematical model in which there are a number of inputs and a number of outputs. The inputs correspond to one or more of the parameters contained within the data packet, such as blood glucose level, respiration level, and amount of drug remaining. The outputs correspond to optimized settings for the operational parameters.

In an example of parameter adjustment, a data packet value is compared by the processor 96 to a reference data packet value stored in the memory 97, and the at least one operational parameter is adjusted based on the comparison. For example, the processor 96 compares a specific data packet value corresponding to a value of a parameter contained within the data packet and compares that value to a reference value stored in the memory 97. The reference value can be an average value of a user parameter measured over a period of time, for example, the reference value could be a user's average or baseline blood glucose level. Alternatively, the reference value can be a predetermined average or desired value obtained from a population of users, obtained from one of the central computer systems 700 connected to the network 702, or from the memory 97 of the drug administration device 500. In general, the reference value relates to a physiological, psychological, or environmental condition of a user. The difference between the data packet value and the reference value is inputted into the adjustment algorithm or looked up in a look-up table to determine how one or more operational parameters should be changed.

In another example of parameter adjustment, a plurality of data packet values are compared to a plurality of reference data packet values, and the at least one operational parameter is adjusted based on the plurality of comparisons. For example, a user's heart rate can be compared to an average heart rate, and a user's blood glucose level can be compared to an average blood glucose level. The adjustments of the operational parameters are based on the combination of comparisons. In general, considering more parameters (e.g., more comparisons) improves the accuracy and reliability of the adjustments to the operational parameters. This improvement is because results from different parameters can be corroborated against each other to check their reliability.

The reference data packet values relate to at least one of a physiological condition of a user, a psychological condition of a user, and an environmental condition of a user. Considering any one or any combination of physiological, psychological, or environmental conditions of a user may increase the accuracy of the adjustment of the operational parameter because these conditions are user specific and therefore are highly relevant to the user's health condition and how the operation of the drug administration device 500 should be effected to achieve optimal health benefits.

The drug administration device 500 being configured to adjust at least one operational parameter based on the combined data allows the patient to be provided with a drug administration device that is able to calculate an optimum dosage to be administered to the patient. Accurately determining the optimum dosage can improve general patient wellbeing, prevent the patient entering a medical crisis, can prevent the drug from running out entirely before replacement drug(s) can be delivered to the patient, and/or can prevent the patient from having to ration the remaining drug and thus not receive as much drug as needed.

The communications interface 99 can send the data packet to one or more of the computer systems 700 and external devices 900 for display on a display device thereof. The display device can then display information related to the data packet, which may enable the user, the user's car provided, and/or a medical practitioner to view information relevant to the user's health and status of the drug administration device 500 to improve knowledge and analysis of the user's care.

After the data has been combined 1050, and either before or after the parameter adjustment 1060, the drug administration device 500 communicates 1070, e.g., using the communications interface 99 thereof, the combined data, e.g., the single data packet, to at least one of the central computer systems 700 in the drug administration system. The central computer system(s) 700 that receive the combined data can implement a compliance monitoring system or Electronic Health Record (EHR) to enable a user's compliance with, and response to, drug administration to be monitored remotely and automatically. The central computer system(s) 700 that receive the combined data may possess more computing power than the processor 96 of the drug administration device 500, so the computer system(s) 700 receiving the combined data can allow faster processing or more advanced models for operational parameter adjustment to be considered. In general, monitoring compliance with the guidance that is associated with the drugs that are administered to a patient in various dosage forms may provide assurance that correct procedures are being followed and avoids the adoption of incorrect and potentially dangerous approaches. Compliance monitoring also enables optimization of the administration of the drug to the patient.

Communicating 1070 the data packet to the at least one central computer system 700 enables the implementation of a safety feature in the drug administration system. The at least one central computer system 700 can analyze the data packet, for example to check that the user has performed a blood glucose test, and only then send an administration signal to the drug administration device 500 that triggers delivery of a dose of the drug from the drug administration device 500. The processor 96 of the drug administration device 96 can be configured only to allow administration of the drug upon receipt of the administration signal. The drug administration being triggered only by receipt of the administration signal may ensure that the user cannot administer the drug from the drug administration device 500 without first performing a test and may prevent the administration of the drug from the drug administration device 500 where the condition of the user renders it unsafe to do so. Preventing administration of a drug from the drug administration device until an administration signal is received may reduce the possibility of a drug overdose occurring and may ensure that the drug is only administered from the drug administration device 500 at suitable times and/or at suitable places (e.g., geographic locations). The prevention of the administration of drug can be effected using the device operation prevention mechanism 40 or 25 that can be selectively activated, e.g., enabled in a default state and disabled by processor 96 instruction in response to receipt of the administration signal. The drug administration device 500 can be equipped with an override feature, for example a button, switch, etc., to enable the user to administer the drug when connection to the at least one central computer system 700 is not possible, e.g., by actuating the override feature to allow for drug delivery from the drug administration device 500.

The at least one central computer system 700 can be configured to transmit the administration signal to the drug administration device 500 automatically in response to analysis of the data packet indicating that administration is proper, e.g., because the user had performed a required blood glucose test. Alternatively, the at least one central computer system 700 can be configured to transmit the administration signal to the drug administration device 500 in response to analysis of the data packet indicating that administration is proper only after manual confirmation that the administration is proper.

FIG. 10A illustrates one embodiment of the central computer system 700 as a smartphone configured to communicate the administration signal wirelessly 701 in response to manual input provided to the central computer system, e.g., by user touch of a start button 703 on a touch screen 705 of the central computer system 700. The start button 703 can be provided via an app installed on the smartphone, as will be appreciated by a person skilled in the art. The manual input in this illustrated embodiment requires touch of the start button 703 for a minimum of five seconds, which helps ensure that the start button 703 is not accidentally touched to cause the start of drug delivery via transmission of the administration signal. The central computer system 700 can have other configurations, and discussed herein, and the manual input can be provided in other ways, such as via voice command to the central computer system 700, keypad input, button depression, etc. The smartphone of FIG. 10A can be used in other ways in connection with embodiments of drug administration devices 500, as described herein.

FIG. 10B illustrates one embodiment of the drug administration device 500 configured to receive the administration signal wirelessly 707 from the central computer system 700 of FIG. 10A or other embodiment of the central computer system 700. The drug administration device 500 in this illustrated embodiment is an injection device configured to be attached to the patient's arm 709 using straps 711 and/or adhesive. Other types of drug administration devices 500, e.g., inhaler, nasal spray device, etc., can be similarly configured to receive the administration signal wirelessly from the central computer system 700 of FIG. 10A or other embodiment of the central computer system 700.

FIG. 10C illustrates another embodiment of the drug administration device 500 configured to receive the administration signal wirelessly 713 from the central computer system 700 of FIG. 10A or other embodiment of the central computer system 700. The drug administration device 500 in this illustrated embodiment is an autoinjector. Other types of drug administration devices 500, e.g., inhaler, nasal spray device, etc., can be similarly configured to receive the administration signal wirelessly from the central computer system 700 of FIG. 10A or other embodiment of the central computer system 700. FIG. 10C also illustrates the dispensing mechanism 20 as including a motor 715; the drug holder 10 in the form of a syringe 717; and the communications interface 99 in the form of a communications chip 719.

The at least one central computer system 700 can determine that one or more of the operation parameters of the drug delivery device should be adjusted based on the data packet in the same way as discussed above with respect to the processor 96 of the drug delivery device 500.

Each of the at least one central computer system 700 and the processor 96 can determine, based on the data packet, that at a drug refill, emergency services, a user reminder prompt, and/or a variation in at least one of the operational parameters of the drug administration device is required. For example, the parameters of the data packet may indicate that an amount of remaining drug is running low in the drug administration device 500 (e.g., by the measured amount of drug being below a predetermined threshold amount) and/or that the user requires an increased dosage (e.g., by the user's blood glucose level being outside an expected range after drug delivery, by the user's blood pressure being outside an expected range after drug delivery, etc.), and therefore a drug refill is necessary and/or that a user reminder prompt to order a prescription refill is needed. Monitoring drug usage based on the data packet enables the central computer system to ensure that the user does not run out of drugs by ensuring timely delivery to the user of replacement drugs and enables adjustment of the parameters of the drug administration device to ration the remaining drug to prevent the drug running out entirely before replacement drugs can be delivered to the user.

For another example, parameters of the data packet may indicate that a maximum number of doses have been delivered from the drug administration device but that one or more additional dose deliveries are needed. The operational parameters of the drug administration device can include a maximum number of doses that can be delivered from the drug administration device before the drug administration device 500 is put into a locked state in which drug delivery from the drug administration device 500 is prevented. The maximum number of doses may be for patient safety (e.g., to help prevent overdosing) and/or to reflect the particular size drug holder 10 of the drug administration device 500 since drug holders can hold different amounts of drug with larger drug holders generally being able to provide more drug doses than smaller drug holders.

In some instances, a drug administration device may malfunction and not properly deliver a dose despite the device being actuated to deliver the dose. In some instances, a drug administration device may be accidentally actuated, such as by an inhaler being caused to deliver drug before the inhaler is inserted into a patient's mouth or a nasal spray device being caused to deliver drug through its nozzle before the nozzle is inserted into a nostril of a patient. In such instances of device malfunction and/or accidental actuation, the patient may not have received the prescribed amount of drug even though the drug administration device has been actuated at least once to deliver the prescribed amount of drug. It may therefore be beneficial to increase the maximum number of allowable doses, e.g., from one to two, from two to three, from two to four, from three to four, from four to five, from four to six, etc., to allow for the prescribed amount of drug to actually be delivered to the patient. An input can be provided to the drug administration device 500, e.g., via the user interface 80, that one mis-actuation occurred, which can trigger the parameters of the data packet to indicate that the maximum number of doses have been delivered from the drug administration device and that one additional dose delivery is needed. In other embodiments, the input can indicate a number of mis-actuations occurred that is greater than one and that therefore a corresponding number of additional dose deliveries are needed. However, limiting requests for additional dose deliveries to one may help prevent drug abuse and/or may allow for more health care professional intervention with the user, e.g., to assess whether additional training is needed if more than one accidental actuation is input as having occurred before the additional dose(s) are authorized, to assess whether device malfunction requires a new device instead of additional dose(s) from the device that malfunctioned, etc. Instead of the input being provided to the drug administration device 500 indicating that one mis-actuation (or more) occurred, the input can be otherwise provided, such as to a preauthorized external device associated with the user, such as the user's smartphone, smart watch, tablet, etc. or a smart device issued to the user by a health care facility where the user is administering the drug.

As discussed above, the data packet indicating that one or more additional doses are needed can be transmitted to the at least one computer system 700 from the drug administration device 500. In some embodiments, the data packet indicating that one or more additional doses are needed can instead or additionally be transmitted to the at least one computer system 700 from another source. For example, some drugs, such as esketamine, ketamine, and other controlled substances, require use of a form such as a patient monitoring form for the particular drug's Risk Evaluation and Mitigation Strategies (REMs). To help track accidental actuations, an entry can be added to the form for a user to indicate whether an accidental actuation of the drug administration device 500 occurred and, if so, how many accidental actuations occurred. Such forms are typically maintained electronically at a computer system, e.g., in a memory thereof. If the entry indicates that at least one accidental actuation occurred, the computer system storing the form can be configured to automatically transmit a notification to the at least one computer system 700 that is in electronic communication with the drug administration device 500. The notification can include the data packet indicating that one or more additional doses are needed, with the number of additional doses needed corresponding to the number entered into the form. Including accidental actuation information in the form may not only facilitate patients receiving full doses as prescribed by allowing for additional drug dose actuation(s) as discussed herein but also may facilitate tracking and analysis of accidental actuations for a particular type of drug administration device 500 and/or for a particular drug by allowing comparisons to be made among multiple patients' form entries indicating whether an accidental actuation of the patient's drug administration device 500 occurred.

In another example, parameters of the data packet may indicate that a user's blood glucose level has fallen low or dangerously low (e.g., by the user's blood glucose level being outside an expected range), and therefore that the user should be prompted, e.g., via the drug administration device's user interface 80, to administer a dose of insulin from the drug administration device 500 or that emergency services are required. If necessary, the processor 96 communicates these signals to the at least one central computer system 700. The at least one central computer system 700 is able to take appropriate action, for example, alerting a medical practitioner, notifying the emergency services, or ordering a drug refill to be sent to the user. Thus, monitoring the health of the user based on the data packet enables the emergency services to be sent to the user if the data packet indicates that the user is in a medical crisis even if the user is incapable of summoning them. In less serious situations, monitoring the health of the user enables a prompt to use the drug administration device to be sent to the user or an increased dosage to be delivered to the user to prevent a medical crisis occurring if the data packet indicates a deterioration of the user's condition.

Where the processor 96 of the drug administration device 500 determines, based on the data packet, that a drug refill or the emergency services are required, it communicates a status signal of the drug administration device to the at least one central computer system 700.

Networking with Other Implantable Systems

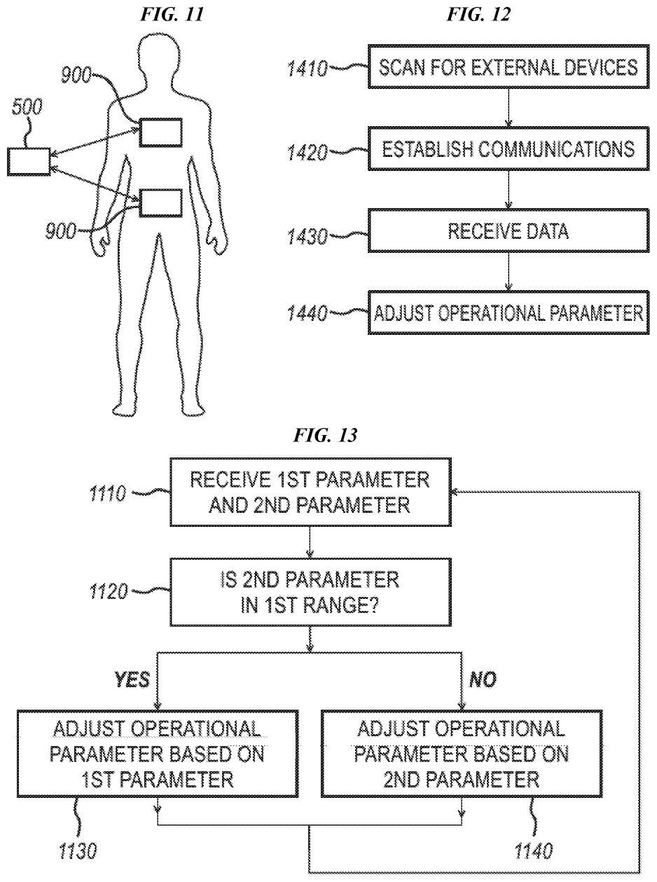

An exemplary embodiment of a drug administration system is shown in FIG. 11 that includes a drug administration device 500 (FIG. 5B) and a plurality of implantable external devices 900 that are shown in FIG. 11 implanted in a patient. The implantation can be fully within the patient or with only a partial portion of the external device 900 implanted within the patient. Although two external devices 900 are shown in FIG. 11, another number of external devices 900 can be present in the drug administration system, e.g., one, three, four, etc. The implanted locations of the external devices 900 in FIG. 11 are example locations, as the external devices 900 can each be implanted in a variety of locations depending on their particular use and configuration. The external devices 900 are considered to be “external” despite being internal to the patient because the external devices 900 are external to the drug administration device 500. The communications interface 99 of the drug administration device 500 is configured to scan for and establish communications with the external devices 900. As discussed above with respect to FIG. 9, each of the external devices 900 is generally configured as a computer system as discussed above with respect to FIG. 8 and, as also discussed above, can be specifically designed for communication with the drug administration device 500 or can be configured with any number of devices, including the drug administration device 500. As also discussed above, examples of the external devices 900 include fitness wearables, heart rate monitors, blood glucose monitors, and dialysis machines.

Each of the external devices 900 is equipped with one or more sensors that is configured to measure a user physiological parameter, which as discussed above relate to a condition of the user of the drug administration device 500 (e.g., the patient having the external devices 900 implanted therein). The one or more sensors of each external device 900 is each configured to record one or more user physiological parameters as user physiological data. Examples of user physiological parameters that the one or more sensors can be configured to measure include blood glucose level, heart rate, heart rhythm, heart rate variability, blood pressure, fatigue level, respiration level, and concentration of a drug in blood. These user physiological parameters can be used, e.g., by the drug administration device 500 and in particular the processor 96 thereof, to assess when more of the drug should be delivered from the drug administration device 500 and to determine how the user is responding to the current drug treatment. For example, a drop in blood glucose level may require an adjustment of the drug dosage. In another example, user physiological parameter(s) may indicate that the user is becoming immune to the drug or is allergic to the drug and that, therefore, corresponding adjustments should be made to the drug dosage or that use of the drug should be ceased with another drug being prescribed to the user that the user is not immune or allergic to.

A particular class of external devices 900 are implantable devices, that is, devices that are suitable for insertion into the human body. Examples of implantable external devices 900 include subcutaneous glucose sensors, subcutaneous heart rate monitors, esophageal LINX® devices, pyloric LINX® devices, brain activity sensors, and subcutaneous motion sensors. Using user physiological data measured from an implanted external device 900 enables determination of an optimum dosage based on data that is hard to measure with non-implanted external devices.

Esophageal and/or pyloric LINX® devices can be used to measure when and how much a user eats and drinks. A LINX® device includes a ring of magnets arranged around the esophagus or pylorus. A sensor placed on one of the magnets can detect a magnetic field produced by the magnets. Changes in the magnetic field caused by movements of the magnets as food and/or liquid passes through the esophagus are measured by the sensor and can be correlated with a type of meal consumed, as will be understood by a person skilled in the art. An amount of expansion and contraction can distinguish between solids and liquids being swallowed. This data (the type of meal) can be used as an indicator of blood glucose level. Hence, data from the LINX® device, which effectively functions as a meal detection sensor, can be used to control, for example, insulin doses administered by the drug administration device 500 as an insulin pump (e.g., the infusion pump 200 of FIG. 2).

As discussed above, the external devices 900 each include a communications interface configured to communicate with the drug administration device 500, e.g., the communications interface 99 thereof. The communications interfaces of the external devices are configured to communicate user physiological data gathered by the one or more sensors of the external devices 900 to the drug administration device 500. As also discussed above, the external device 900 each include a processor configured to control the sensor(s) thereof and the communications interface thereof, and can each include a memory configured to store the user physiological data measured by the sensor(s) thereof permanently or temporarily.

An exemplary embodiment of a method of operating the drug administration device 500 of FIG. 11 is shown in FIG. 12. In general, the method may allow for actively seeking to obtain information relevant to drug administration and thereby enable use of that information for a purely objective determination of an optimum setting of the operational parameter(s) of the drug administration device 500 and lead to appropriate adjustment of the operational parameter(s) of the drug administration device 500. The operational parameters and the adjustment thereof is achieved as discussed above.

As shown in FIG. 12, the communications interface 99 of the drug administration device 500 scans 1410 for external devices to discover or identify external devices 900 that are available for communications, similar to that discussed above with respect to FIGS. 9 and 10. Having identified available external devices 900, the drug administration device 500 establishes communications 1420 with the identified available external devices 900 which, as also discussed above with to FIGS. 9 and 10, includes the communications interface 99 attempting to connect to the identified available external devices 900. Establishment 1420 of a communications link with the identified available external devices 900 is either automatic or requires a user input (e.g., a user input to the user interface 80 of the drug administration device 500) to authorize connection. Manual establishment of communication may enable enhanced security and user control of the parameters used to control the dosage, e.g., control of the dosing scheme of the drug administration device 500. Automatic establishment of communication with an external device 900 on occasions subsequent to a manual establishment of communication with that same external device 900 may improve efficiency in establishing communication and may reflect that the external device 900 has already been established as a trusted external device. The identification of any trusted external devices can be stored in the memory 97 of the drug administration device 500. Accordingly, the first time the drug administration device 500 attempts to connect to a particular external device 900, user authorization can be required to establish a connection, and then subsequent connections with the particular external device 900 the communications link can be established automatically.

In an exemplary embodiment, the drug administration device 500 seeks to establish communications 1420 with as many external devices 900 as possible in order to gather the maximum amount of data relating to as many user physiological parameters as possible. Similar to that discussed above with respect to FIGS. 9 and 10, this maximizing of connections may enable the drug administration device 500 to discover and connect to all available external devices 900 that may be measuring data relevant to drug administration using the drug administration device 500, e.g., relevant user parameter data, which may enable accuracy of a determination of an optimum dose to be improved. The communications interface 99 either communicates with many external devices 900 simultaneously or continuously loops through a list of individual, consecutive connections with the available external devices 900, as will be appreciated by a person skilled in the art. The drug administration device 500 can connect to one or more arrays of external devices 900, for example arrays of motion sensors attached to the user.

Also similar to that discussed above with to FIGS. 9 and 10, the established 1420 communication is wireless.

After communication is established 1420, the drug administration device 500 receives 1430 user physiological data from the external devices 900, e.g., by the external devices' communications interfaces transmitting data received by the communications interface 99 of the drug administration devices. Similar to that discussed above with respect to FIGS. 9 and 10 and the sending of ancillary data, the user physiological data can be sent automatically by the external devices 900, or user physiological data can be sent upon request by the drug administration device 500. Different ones of the external devices 900 can have automatic and requested communication.

The drug administration device 500 can receive 1430 user physiological data from multiple ones of the external devices 900 with which the drug administration device 500 has established communications such that the drug administration device 500 receives different types of user parameter data from each of the external devices 900, and/or one or more of the external devices 900 can be configured to measure multiple user physiological parameters such that the drug administration device 500 can receive 1430 multiple types of user physiological data from a same external device 900. Therefore, the user parameter data received at the drug administration device 500 can relate to multiple user physiological parameters. The processor 96 of the drug administration device 500 identifies the one or more user parameters, e.g., blood glucose level, heart rate, etc., that the user physiological data relates/corresponds to. This identification can be achieved by checking a marker signal associated with each parameter in the user physiological data. The marker signal uniquely identifies a particular parameter as being a particular type of user physiological data, e.g., blood glucose level, heart rate, etc. Identifying what user parameter the user physiological data relates to enables improved control of the operational parameters by the drug administration device 500 due to better understanding of the input data.

After the user physiological parameter has been identified, the user can be prompted, for example via a notification on a display screen or other user interface 80 of the drug administration device 500, to confirm via user input that the identified user parameter should be used to adjust 1440 operational parameter(s) of the drug administration device 500. This confirmation enables the user to make an informed decision about which parameter the drug administration device uses to control the dosage. However, in at least some instances, user control of whether to adjust the dosing scheme by adjusting 1440 one or more of the operational parameters may not be desirable due to, e.g., uncertainty in the user's knowledge level regarding the drug administration device 500 and/or the drug therein, urgency in addressing certain sensed data by adjusting the dosing scheme in a timely fashion that is achievable by automatic adjustment but not necessarily when first requiring confirmation, etc.

After receiving 1430 the user physiological data, and after having received confirmation of parameter adjustment if required, the processor 96 of the drug administration device 500 adjusts 1440 the operational parameters of the dosing scheme based on the user physiological data. The adjustment of 1440 of the operational parameters is achieved similar to that discussed above regarding the adjustment 1060 of FIG. 10. In general, the processor 96 determines whether and by how much, and which one or more of, the operational parameters should be changed so that the drug administration device 500 operates in an optimal way based on the user physiological data, which may improve the operation of the drug administration device 500.

FIG. 13 illustrates an embodiment of a method of receiving parameter data and adjusting an operational parameter. The method of FIG. 13 is described with respect to the embodiment of FIGS. 11 and 12 but similarly applies to receiving parameter data and adjusting an operational parameter with respect to the embodiment of FIGS. 9 and 10.

User physiological data is received 1110 at the drug administration device 500 and includes a first user physiological parameter and a second user physiological parameter. The first user physiological parameter and the second user physiological parameters can be received from the same external device 900 or from separate external devices 900. The first and second user physiological parameters relate to different physiological parameters. For example, the first user physiological parameter can relate to a blood glucose level whereas the second user physiological parameter can relate to a heart rate.

The operational parameter is adjusted based on either the first user physiological parameter or the second user physiological parameter, which enables the drug administration device 500 to control the operational parameter, for example optimal dosage, based on the most suitable of two different user physiological parameters. It will be appreciated that there can be more than two user physiological parameters and that the at least one operational parameter can be adjusted based on the more suitable of these three of more user physiological parameters similar to that discussed herein with respect to the first and second user physiological parameters.

The processor 96 assesses 1120 whether the second user physiological parameter is in a first range of parameters value. For example, the processor 96 can assess whether the second user physiological parameter is above or below a threshold value. The threshold value can be a standard value for that user physiological parameter stored in the memory 97 of the drug administration device 500. The threshold value can be based on prior research to ensure that the most suitable of the two user physiological parameters is selected for controlling the operational parameter. For another example, the processor 96 can assess whether the second user physiological parameter is within a threshold range. The threshold range can be a range of standard values for that user physiological parameter stored in the memory 97 of the drug administration device 500. The threshold range can be based on prior research to ensure that the most suitable of the two user physiological parameters is selected for controlling the operational parameter.

If the second user physiological parameter is in the first range, for example below the threshold value, above the threshold value, or within the threshold range as appropriate for that particular user parameter, then, the operational parameter is adjusted 1130 by the processor 96 based on the first user physiological parameter. That is, the processor 96 controls the dosage using a first algorithm or look-up table with the first user physiological parameter as an input.

If the second user physiological parameter is not in the first range, e.g., the second user physiological user parameter is in a second range, then, the operational parameter is adjusted 1140 based on the second user physiological parameter. That is, the processor 96 controls the dosage using a second algorithm or look-up table with the second user physiological parameter as an input.

The first and second algorithms or look-up tables differ since they are functions of different variables. The first algorithm or look-up table has the first user physiological parameter as the input value, whereas the second algorithm or look-up table has the second user physiological parameter as the input value.

Depending on the second user physiological parameter, the first range and the second range can independently be an open-ended range or a closed range. The first range and the second range can independently be a continuous range or a plurality of discrete ranges. The first range and the second range can independently be a single or a plurality of point values. The threshold value may be equal to a limit of the first range, and the threshold value may be a boundary of the second range.

After the operational parameter is adjusted based on the first parameter 1130 or on the second parameter 1040, the method loops back to receiving 1110 more user physiological data.

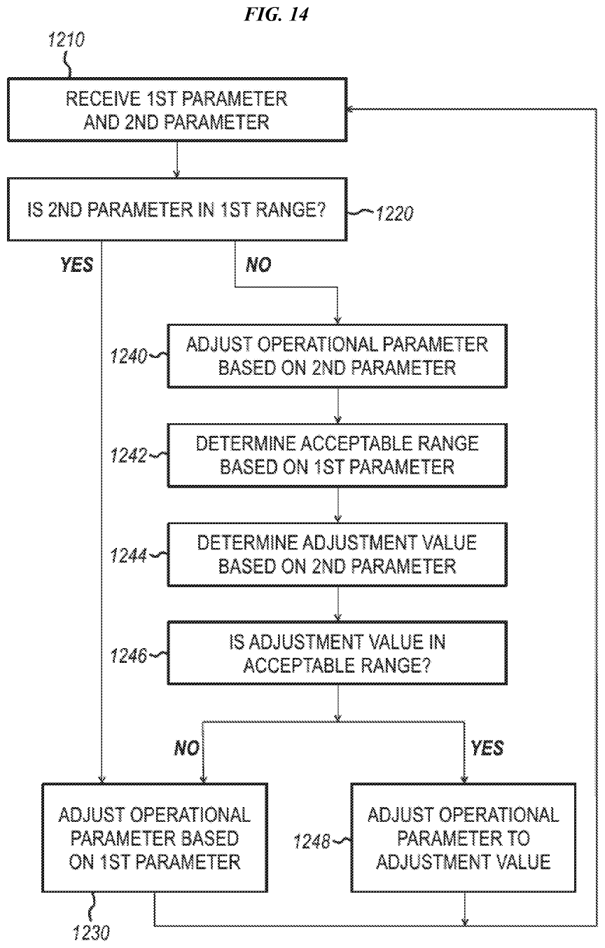

FIG. 14 illustrates another embodiment of receiving parameter data and adjusting an operational parameter. The method of FIG. 14 is described with respect to the embodiment of FIGS. 11 and 12 but similarly applies to receiving parameter data and adjusting an operational parameter with respect to the embodiment of FIGS. 9 and 10.

The method of FIG. 14 is the same as that described with respect to the method of FIG. 13 with the following exceptions. In the case that, after receiving 1210 the first and second user physiological data, it is determined 1220 that the second user physiological parameter is in the second range (e.g., is not in the first range), the operational parameter is adjusted 1240 based on the second user physiological parameter. In this case, adjusting 1240 the operational parameter based on the second user physiological parameter includes the processor 96 determining 1242 an acceptable range of adjustment values of the operational parameter based on the first user physiological parameter. That is, the processor 96 uses an algorithm to determine a range of values within which it would be safe and effective to set the operational parameter. The processor 96 also determines 1244 an adjustment value of the operational parameter. In other words, the processor 96 calculates the value to which the operational parameter would be set if nothing else were considered. The processor 96 assesses 1246 whether the adjustment value, based on the second user physiological parameter, is within the acceptable range of adjustment values. If the adjustment value is determined 1246 to be within the acceptable range of adjustment values, then the operational parameter is set 1248 to the adjustment value. On the other hand, if the adjustment value is determined 1246 to be outside the acceptable range of adjustment values, control of the operational parameter is adjusted 1230 based on the first similar to the adjustment 1130 discussed above. The method of FIG. 14 helps ensure that if one of the user physiological parameters is initially determined to be the most suitable for controlling the device 500, this determination is checked before the adjustment is implemented so that if the proposed adjustment is actually deemed an unsuitable operational parameter change, the control is switched to the other parameter, or a combination of the parameters, that is more suitable.

In an example of the method of FIG. 14, an amount insulin administered by the drug administration device 500 as an insulin pump (e.g., the infusion pump 200 of FIG. 2) is primarily controlled by a blood glucose level of the user. The heart rate can thus be used as the first user physiological parameter. However, when the user is undergoing periods of high physical activity, that level of activity can drive metabolic needs of the user rather than normal body processes. In this case, heart rate (e.g., the second user physiological parameter) as an indicator of activity level can be used as the primary control. The insulin pump can switch from blood glucose control to heart rate control when the heart rate is above a particular level (threshold value). When the heart rate is above the threshold value, e.g., is not in the first range, an acceptable range of adjustment values of the insulin dosage is determined based on the blood glucose level. The insulin dosage is then controlled within that range by the heart rate. Should the heart rate instruct an insulin dosage value outside the acceptable range of adjustment, the insulin pump restores control of the insulin dosage to the blood glucose level or a combination of blood glucose level and heart rate.

In another embodiment of a method of receiving parameter data and adjusting an operational parameter, both the first user physiological parameter and the second user physiological parameters are used to adjust the operational parameter, which enables control based on both of the first and second user physiological parameters if this is the most optimal. For example, an implanted brain activity sensor (e.g., a first one of the implanted external devices 900) can track brain activity for seizure detection and an implanted motion sensor (a second one of the implanted external devices 900) in a limb of the user can detect tremors. The brain activity sensor and the motion sensor both communicate with the drug administration device 500. The processor 96 of the drug administration device 500 can therefore assess whether brain activity is causing a tremor or seizure and adjust the operational parameter, and thus the dosing scheme, accordingly.

### Remotely Updating the Drug Administration Device

It may be desirable to provide remotely connectable, drug administration devices. Such drug administration devices allow for some degree of remote operation of the drug administration device. However, remotely updating a drug administration device poses a number of problems. Firstly, while remotely updating a device may be a more convenient way to update drug treatments, doing so may increase the risk of a security breach since data is being transmitted to the device from another device over a network where the data may be intercepted and/or altered by an unauthorized party. Avoiding interception and alteration of data may be especially important for drug administration devices because if data involving drug administration devices are hacked, a quantity or frequency of drug administration to a patient can be illicitly and/or dangerously varied from an intended quantity or frequency, having life-threatening consequences to the patient.

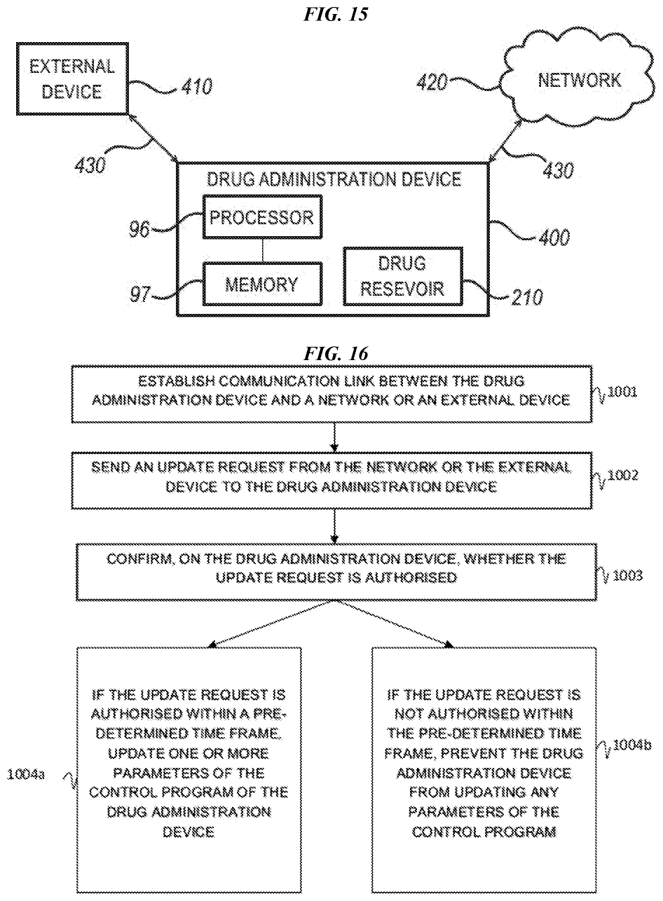

FIG. 15 illustrates an embodiment of a drug administration system including a drug administration device 400 (FIG. 5A) configured to be remotely updated. As mentioned above, FIG. 5B illustrates an exemplary embodiment of the device 501 as drug administration device 500. FIG. 15 thus shows the drug administration device 501 as including the processor 96 (FIG. 5B) and the memory 97 (FIG. 5B). The drug administration device 501 can have any of the forms of drug administration devices disclosed herein, including an autoinjector (e.g., the autoinjector 100 of FIG. 1), an infusion pump (e.g., the infusion pump 200 of FIG. 2), an inhaler (e.g., the inhaler 300 of FIG. 3), or a nasal spray device (e.g., the nasal spray device of FIG. 4). The drug administration device 501 in this illustrated embodiment is an infusion pump and thus is shown in FIG. 15 as including a drug holder in the form of a drug reservoir 210 (FIG. 2).

The drug administration system also includes a network 420 and an external device 410. The network 420 is configured to allow communication therethrough with one or more computer systems similar to the network 702 of FIG. 7 configured to allow communication therethrough with one or more computer systems 700. The drug administration device 400 is configured to communicate with the computer system(s) via the network 420 over a communication link 430. The communication link 430 between the drug administration device 400 and the computer system(s) via the network 420 is shown in FIG. 15 as being bi-directional but can instead be uni-directional to the drug administration device 400. The communication between the drug administration device 400 and the computer system(s) can be initiated by either the computer system(s) or the drug administration device 500. The communication can be established between the drug administration device 400 and the computer system(s) in any of a variety of ways, as discussed above, and can include establishing secure communication, as also discussed above. The drug administration device 500 can be configured to, in response to a security protocol indicative of failing to establish communication with the computer system(s) over the communication link 430, communicate an error message to a user, e.g., via an indicator of the device 400 or a user interface of the device 400.

The external device 410 is external to the drug administration device 400 and is similar to the external device 900 discussed above. The external device 410 is configured to communicate data over a wireless link 430 to the drug administration device 400, e.g., the wireless link 430 is uni-directional with the external device 410 being configured to broadcast data to the drug administration device 400 without expecting or being able to receive a response from the drug administration device 400 over the link 430. In such a case, the drug administration device 400 includes a communications interface that is not required to transmit data to the external device 410 (e.g., to a communications interface thereof) but only to receive data from the external device 410. Alternatively, the communication link 430 between the external device 410 and the drug administration device 400 can be bidirectional (as shown in FIG. 15), or, in other words, can be a two-way link, such that data can be sent across the link 430 in either direction. The communication between the drug administration device 400 and the external device 410 can be initiated by either the external device 410 or the drug administration device 400. The communication can be established between the drug administration device 400 and the external device 410 in any of a variety of ways, as discussed above, and can include establishing secure communication, as also discussed above. The drug administration device 500 can be configured to, in response to a security protocol indicative of failing to establish communication with the external device 410 over the communication link 430, communicate an error message to a user, e.g., via an indicator of the device 400 or a user interface of the device 400.

Depending on the particular scenario being implemented, communication can be attempted to be established between the drug administration device 400 and each of the network 420 and the external device 410, between the drug administration device 400 and the network 420 but not the external device 410, or between the drug administration device 400 and the external device 410 but not the network 420. For example, if no computer system(s) are being used, then communication need not be established between the drug administration device 400 and the network 420. Additionally, although only one network 420 and one external device 410 is shown in FIG. 15, the drug administration system can include a plural number of networks 420 and/or a plural number of external devices 410.

In an exemplary embodiment, the communication link 430 is established between the drug administration device 400 and either or both of the network 420 (for communication over the link 430 with one or more computer systems via the network 420) and the external device 410. Establishing the link 430 includes sending a digital certificate from the network 420 (e.g., from a computer system communicating using the network 420) or the external device 410 to the drug administration device 400. The digital certificate includes a digital signature, which can be used to verify whether the attempted communication is valid. The drug administration device 400 is configured to confirm the identity of the digital certificate for the network 420 in order to authorize the communication link 430 for the network 420 and to confirm the identity of the digital certificate for the external device 410. in order to authorize the communication link 430 for the external device 410. A communication including a digital signature can similarly be exchanged from the drug administration device 400 to the external device 410 and/or the network 420.

If the digital signature cannot be validated, the communication link 430 will not be established. In other words, any communication between the drug administration device 400 and the external device 410 will be entirely terminated if the external device's digital certificate cannot be validated. Until the communication link 430 can be established between the external device 410 and drug administration device 400, no further data can be sent to the drug administration device 400 from the external device 410. If the communication link 430 is established between the drug administration device 400 and the external device 410, the drug administration device 400 is configured to be responsive to update requests from the external device 410. In general, an update request requests a change of at least one parameter of the drug administration device 400 that is stored in the memory 97, with the update request including an instruction of the change to implement. After an update request is received on the drug administration device 400 from the external device 410 and appropriately verified, the processor 96 of the drug administration device 400 is configured to modify one or more of the parameters stored in the memory 97 in any of the ways discussed above with respect to adjusting a parameter or in another way, such as a direct request to change a parameter to a particular, specified value that the processor 96 can effect. For example, the drug administration device 400 can be instructed via an update request from the external device 410 to change a parameter representing a duration of drug administration from one value to another value, e.g., ten seconds to twenty seconds. One or more parameters can be updated in response to a single update request. The processor 96 of the drug administration device 400 can be configured to store in the memory 97 a digital characteristic of the communication link 430 associated with the external device 410 and/or the update request from the external device 410 if the update request is not verified within a predetermined time frame.

The update request may not be to update a parameter of the drug administration device's dosing scheme but be another type of parameter update request. For example, the update request can be a request relating to updating firmware and/or software of the drug administration device's control algorithm that controls general operation of the drug administration device 400, e.g., operation of the device 400 that is not part of the dosing scheme. The control algorithm is configured similar to other algorithms discussed herein and can have its parameters similarly changed by the processor 96. For example, the drug administration device 400 can be instructed via an update request from the external device 410 to change a parameter of the control algorithm representing a maximum number of allowable doses deliverable from the drug administration device 400 from one value to another value, e.g., two dose deliveries to three dose deliveries.

Similarly, any communication between the drug administration device 400 and the network 420 will be entirely terminated if the network's digital certificate (e.g., computer system's digital certificate) cannot be validated. Until the communication link 430 can be established between the network 420 and drug administration device 400, no further data can be sent to the drug administration device 400 from the network 420. If the communication link 430 is established between the drug administration device 400 and the network 420, the drug administration device 400 is configured to be responsive to update requests from the network 420 (e.g., from a computer system communicating with the drug administration device 400 through the network 420). After an update request is received on the drug administration device 400 from the network 420 and appropriately verified, the processor 96 of the drug administration device 400 is configured to modify one or more of the parameters stored in the memory 97 in any of the ways discussed above with respect to adjusting a parameter or in another way, such as a direct request to change a parameter to a particular, specified value that the processor 96 can effect. For example, the drug administration device 400 can be instructed via an update request from the network 420 to change a parameter representing a duration of drug administration from one value to another value, e.g., ten seconds to twenty seconds. For another example, the drug administration device 400 can be instructed via an update request from the network 420 to change a parameter of the control algorithm representing a maximum number of allowable drug administrations from one value to another value, e.g., two dose deliveries to three dose deliveries. One or more parameters can be updated in response to a single update request. The processor 96 of the drug administration device 400 can be configured to store in the memory 97 a digital characteristic of the communication link 430 associated with the external device 410 and/or the update request from the external device 410 if the update request is not verified within a predetermined time frame.

The processor 96 of the drug administration device 400 can be configured to place the drug administration device 400 into a locked state in response to a security protocol. The drug administration device is prevented from any wireless communication over the relevant communication link 430 in the locked state.

For example, the security protocol can be when the communication link 430 (whether for the external device 410 or the network 420) fails to be established, which may be indicative of a security breach or other problem.

For another example, the security protocol can be when the communication link 430 is established but a security code is not verified. The security code can include a numeric, alphabetic, or alphanumeric code provided to the user, or a password chosen by the user, and configured to be input by a user to the drug administration device 400 (e.g., via a user interface 80 of the device 400) or by a user to the external device 410. The processor 96 can be configured to compare the input code with a pre-stored code (e.g., pre-stored in the memory 97), with a match indicating verification of the input code and no match indicating non-verification of the input code. The security code can be configured to be input before any drug is delivered from the drug administration device 400. In some embodiments, a plurality of security codes can be provided to a user. The plurality of security codes can be configured to be sequentially used. A first security code can be provided to the user at prescription, such as by being printed on the drug administration device's prescription label or on a receipt or another paper provided upon prescription pickup. A second security code can be provided to the user at a later time after the first security code has been input and verified, e.g., verified before any drug is delivered from the device. The second security code can be required to be input and verified before a first dose of the drug can be delivered from the drug administration device. One or more additional security codes can be provided to the user after each prior security code is input and verified to allow for more than two doses of the drug to be delivered from the drug administration device 400. The second security code, and any additional security codes, can be provided to the user in a variety of ways, such as by being transmitted from the external device 410 to the drug administration device 400 for viewing on a user interface 80 thereof or by being transmitted from the external device 410 to a previously authorized external device associated with the user, such as the user's smartphone, smart watch, or tablet.

For yet another example, the security protocol can be when the communication link 430 is established but a verified input, such as a fingerprint or other biometric input, is not provided to a previously authorized external device associated with the user, such as the user's smartphone, smart watch, or tablet, which may be indicative of someone other than the prescription holder trying to use the drug administration device 400.

An embodiment of a method of transferring data to the drug administration device 400 for updating a parameter is shown in FIG. 16. The communication link 430 is established 1001 between the drug administration device 400 and either or both of the network 420 or the external device 410. An update request is sent 1002 from the network 420 (or the external device 410) to the drug administration device 400. It is confirmed 1003, on the drug administration device 400, whether the update request is authorized. If the update request is authorized within a predetermined time frame, one or more parameters are updated 1004a in accordance with the request. If the update request is not authorized within the predetermined time frame, the drug administration device 400 is prevented 1004b from updating any parameters. Preventing 1004b the drug administration device 400 from updating refers to stopping a parameter of the drug administration device from changing, even in response to receiving an update communication. The predetermined time frame is a length of a time interval (e.g., one minute, ninety seconds, etc.) stored in the memory 97 of the drug administration device 400, e.g., stored as a pre-programmed value. In other words, the drug administration device 400 can require a response within a specific time interval after receiving the update request and can query the predetermined time frame stored in the memory 97 to determine whether a response has been received within the appropriate time. Using the confirmation may ensure that any update request for the drug administration device 400 is properly authorized before updating or changing any parameters and thereby help prevent unauthorized manipulation of the drug administration device 400, which may have life-threatening consequences to the patient to which the drug administration device 400 delivers a drug. In other words, the confirmation 1003 may ensure that, regardless of what computer system or external device 410 initiated the update request, the patient can properly ensure that they authorize the parameter(s) to be updated and thereby help ensure that the patient is aware of any changes instead of the update automatically taking place unknown to the patient.

Confirming 1003 on the drug administration device 400 whether the update request is authorized can include confirming whether an input from a user has occurred on the drug administration device 400. The drug administration device 400 can provide at least one user notification of the received authorized update request, such as by displaying information relating to the authorized update request on a user interface of the drug administration device 400, provide a sound, etc. The notification can prompt the user to review the parameter(s) to be updated (e.g., by reading information on the display, etc.), and, if the update appears to be correct or otherwise acceptable, accept the update. The update can be accepted in a variety of ways, such as by providing an input to a user interface of the device 400 that acknowledges the update, such as by pressing a button, turning off the notification sound, providing a predetermined input on the user interface on a keypad, etc. The predetermined input can be, for example, a specific word such as “accept,” a security code previously provided to the user, etc. Upon acceptance of the update request, the processor 96 can confirm whether acceptance occurred within the required predetermined time frame and, if so, extract the relevant parameter(s) from the update request and modify the corresponding parameter(s) in the memory 97 to match the requested parameter(s). The processor 96 can query the memory 97 after writing the parameter(s) to the memory 97 to ensure that the write procedure was successful and that the parameter(s) were successfully changed in accordance with the update request.

The user may be prompted by the drug administration device 400 to provide an input prior to the drug administration device 400 confirming whether an input from a user has occurred on the drug administration device 400. This prompting may ensure that, when a remote update is not initiated by the user, the user is made aware of the update request so that they can accept or reject the update. Alternatively, the drug administration device 400 can only initiate an update once a user has provided an input on the drug administration device 400 (e.g., pressing a button and/or inputting a previously-provided security code to send an enquiry to the network 420 and/or external device 410 regarding any available updated parameters to download). The authorization of the update request can itself initiate the sending of the update request from the network 420 or the external device 410. Accordingly, the confirmation of whether the update is authorized within the predetermined time frame can include the time preceding the sending of the update request.

Certain security protocols can be initiated if a digital certificate cannot be verified, as this may be indicative of a hacking attempt or other security problem. That is, the drug administration device 400 can be configured to store a digital signature of an attempted communication in the event of a failure to establish the communication link 430 between the network 420 and the drug administration device 400 or between the external device 410 and the drug administration device 400. The drug administration device 400 can be configured to prevent further communication, notify an external system, and/or reset the drug administration device 400 to default parameters after a predetermined amount of failed communication attempts, which can be a single failed communication attempt or a plural number (two, three, four, five, or more than five) failed communication attempts. The default parameters of the device 400 can be manually loaded/configured by a healthcare professional. For improved security, the default parameters can only be updated locally on the device 400 and, once set, cannot be modified without the use of a password or other authenticating equipment locally (e.g., via RFID tag at a hospital or other healthcare facility).

The method can include implementing a security protocol if the update request is not authorized within the predetermined time frame, as this lack of timely authorization may be indicative of a hacking attempt or other security problem. In other words, if the user of the drug administration device 400 is unaware of the attempted update, this may be indicative that the update request is fraudulent. Implementing the security feature may improve security of the drug administration device 400, as well as improve an ability to react appropriately to communications which have previously not been authorized.

The security protocol can cause the drug administration device 400 to respond or function in a certain manner instantly or the security protocol can have levels of escalation so as to cause the device 400 to respond or function in a certain manner only after a certain plural number of failed attempts. In other words, a single failure to authenticate the update request can have no further consequence other than preventing the update, however, multiple failures to authenticate the update request can trigger a response from the drug administration device 400. For example, the security protocol can result in no changes to the drug administration device 400 after a single failed communication attempt but can result in limited functionality after a plurality of failed communication attempts, for example five failed communication attempts.

In general, a security protocol refers to a program that is implemented (e.g., an algorithm that is executed by the processor 96) to escalate the security of the drug administration device 400 when a threat, or a vulnerability, is detected. In other words, the security protocol can be the approach adopted by the drug administration device 400 for dealing with unauthorized update requests or any unauthorized communication received by the drug administration device 400.

The security protocol can include the drug administration device 400 storing in the memory 97 a digital characteristic of the communication link 430 and/or the update request. A digital characteristic generally refers to any feature of the communication, including a digital signature, contents of the message sent, and an original IP address of the sender of the message. Thus, for each unauthorized update request, a digital characteristic of the update request can be stored in the memory 97, e.g., contents of the update request message, an original address where the update request originated, the entire update request message, etc. Storing the digital characteristic may be beneficial to compare a characteristic (such as an attack signature of the communication) in an update request that is being checked for validation against known attacks, stored as digital characteristics in the device 400.

The security protocol can include implementing a counter-measure if the stored digital characteristic matches any of the known digital characteristics in the memory 97 of the device 400. That is, the method for assessing whether to update parameters of the drug administration device 400 in response to a received update request can include comparing the stored digital characteristic against a pre-defined list of known digital characteristics stored in the drug administration device 400, and if the communication attempt matches a known style of attack as indicated by the stored known digital characteristics, the drug administration device 400 can react according to the known characteristic of the attack. An attack signature generally refers to any arrangement of digital information that can be used to identify a particular form of attack attempt to exploit a security protocol. In general, a counter-measure is a specific response in hardware or software that can be implemented by the device 400 (e.g., by the processor 96 executing an algorithm stored in the memory 97) in response to a detected problem. For each type of attack, a specific counter-measure can be associated therewith. That is, if a certain type of attack (e.g., a Distributed Denial of Service, or DDoS) is detected, the drug administration device 400 can respond in a suitable way (e.g., defaulting the device 400 to preset parameters and preventing any further communication). The stored characteristics and corresponding counter measures can only be updated through a secure OEM server, or a secure, trusted network. For example, the processor 96 may query a look-up table of responses stored in the memory 97 that includes a set of instructions for the processor 96 to react in a specific way to a specific type of attack.

The counter-measure can include notifying an external device (but not the external device 420 if the external device 420 is the sender of the request that triggered the counter-measure). The notification may allow the user and/or a healthcare professional to be made aware of potential issues with the patient's drug administration device 400 and take any desired corrective action, e.g., replace the device 400 with a new drug administration device 400, upgrade software of the drug administration device 400 to help prevent future problems, etc. An external system generally refers to any system external to and in wireless communication with the drug administration device 400, including a network or a smart phone. In one example of notifying an external device, the drug administration device 400 is configured to send a wireless communication to a paired device and/or a computer system (accessible via the network 420) to alert a user of an attempted connection attempt. The notification can be communicated via visual, aural, and/or haptic feedback, including a text alert, a change in a display of a device, or a sound from a speaker. The drug administration device 400 itself can notify the user via a device indicator of the device 400 that an unauthorized update request has occurred.

In another example of a counter-measure, after a predetermined number (e.g., one, two, three, four, etc.) failed communication attempts, the drug administration device 400 can be configured to enter a locked state in which the processor 96 is unable to write anything to the memory 97 or receive any further wireless communications. The locked state can be accomplished either via a hardware solution (e.g., an internal switch is opened via a relay to disconnect the drug administration device's communications interface, etc.), or a software solution (e.g., not responding to incoming data detected by the drug administration device's communications interface, etc.). For example, the processor 96 of the drug administration device 400 can require an override from the user interface of the drug administration device 400 before any serial communication containing “write” data can be sent between the processor 96 and the memory 97. That is, after the predetermined number of failed communication attempts, the processor 96 will only be configured to “read” data from the memory 97 and would not be able to modify any of the parameters stored in the memory 97.

The locked state can be temporary and can be reverted once a manual input on the drug administration device 400 is received. That is, the user can manually cause a restart of wireless communication once it has been assured that it is safe to resume remote communication. For example, the manual input can be received via a user input to the drug administration device's user interface, such as by pressing a button, etc. For another example, the manual input can be a fingerprint or other biometric input provided to a previously authorized external device associated with the user, such as the user's smartphone, smart watch, or tablet.

When the drug administration device 400 is placed into a locked state, the user of the drug administration device 400 can be notified, such as via either the user interface of the drug administration device 400 or a display of an external device (e.g., the external device 420 or other external device). The user be given the option via the notification to reinitiate the full operation of the device 400, which would re-enable the wireless transmission of data and re-enable the writing of data to the memory 97 by the processor 96. The drug administration device 400 can be configured to transmit an instruction to provide the notification to the external device prior to entering the locked state to allow a record of the sent instruction to be stored in the memory 97 prior to “write” access to the memory 97 being denied.

Establishing 1001 the communication link 430 can include exchanging a decryption key along with the digital signature. Thus, if the digital certificate can be validated, the drug administration device 400 can have access to the decryption key to decrypt any encrypted data. Unsecured networks may be vulnerable to hacking, which would put the safety of the patient at risk, so providing for encryption/decryption may improve electronic security of the device 400 and physical security of the patient. Any type of encryption (including WEP, WPA, and WPA2 encryption methods) can be used to encrypt the transmitted data, as discussed herein. As also discussed herein, various digital certificate validation schemes and cryptographic protocols, including the Secure Sockets Layer protocol (SSL), the Transport Layer Security protocol (TLS), RSA, or any other public/private key protocols can be utilized to establish the communication.

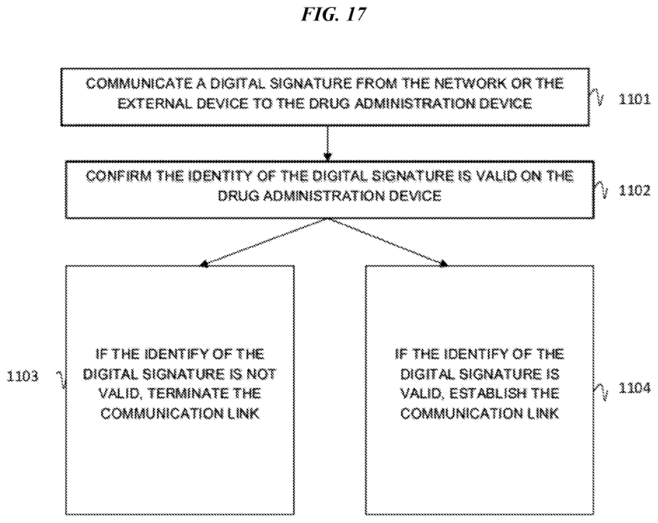

An embodiment of a method for establishing 1001 the communication link 430 between the drug administration device 400 and the network 420 or the external device 410 and a is shown in FIG. 17. A digital signature from the network 420 or the external device 410 is communicated 1101 to the drug administration device 400. In general, a digital signature refers to a mathematical technique to validate the authenticity and integrity of a message based on cryptography. The digital signature can be an electronic sound, symbol, or process attached to or logically associated with a record. An example of a digital signature is a public/private key arrangement, as discussed above, under which communication to the drug administration device 400 is encrypted with a public key but is only decryptable with a private key stored at the drug administration device 400.

An identity of the digital signature is confirmed 1102 as valid or invalid on the drug administration device 400. If the identity of the digital signature is not valid, the communication link 430 is terminated 1103. If the identity of the digital signature is valid, the communication link 430 is established 1104.

Once the communication link 430 is established 1104, data can be transmitted from the associated network 420 or external device 410 to the drug administration device 400 using the established communication link 430. The network 420 or external device 410 can send an update request to update one or more parameters of the drug administration device 400 as discussed above. In other words, the network 420 or external device 410 can send a command to initiate the remote update of the drug administration device 400. This command can include the update request along with the specified update data, or alternatively, may exclusively include the update request. In the former case, the update data can be stored in the memory 97 of the drug administration device 400 until the update request is authorized. If not authorized, this stored update data can be removed. In the latter case, the update data can be sent from the network 420 or external device 410 over the communication link 430 once the update request is confirmed.

Various methods to validate 1002 the update request are possible. This authorization can include a manual authorization on the drug administration device 400. This manual authorization can include a manual user input such as the user providing an input to the user interface of the device 400, pressing a button on the device 400, flipping a switch on the device 400, etc. The manual input can be used to put the drug administration device 400 into a mode in which the device 400 can communicate wirelessly. The user's manual input for the manual authorization can be used to put the drug administration device 400 into a mode to allow remote updating for only a certain amount of time, e.g., three minutes, five minutes, etc. The drug administration device 400 can be configured to prompt the user for the manual input. This prompting can use a device indicator of the drug administration device 400, for example the prompt can be visually indicated on a display aurally indicated with a speaker, etc. The user can have a certain predetermined amount of time (e.g., ten seconds, fifteen seconds, etc.), to accept/authorize the request. The drug administration device 400 can include a timer or a clock electronically connected to the processor 96 to ensure that the manual input is received within the predetermined amount of time. Alternatively to the manual authorization being input to the drug administration device 400, the manual authorization can be input, e.g., via a fingerprint or other biometric input, to a previously authorized external device associated with the user, such as the user's smartphone, smart watch, or tablet.

The authorization of the update request can happen automatically. Automatic authorization can be appropriate, for example, when the drug administration device 400 is being updated through a hospital network in which case a healthcare professional may be presumed to be causing the update of the control parameter(s) such that the drug administration device 400 need not require a manual authorization before the updating. For another example, automatic authorization can be appropriate when updating using the patient's own external device 410, e.g., the patient's own smart phone, the patient's own tablet, etc., after an initial pairing of the drug administration device 400 and the patient's own external device 410. In the case of automatic authorization, the drug administration device 400 can automatically verify and authorize the update request without need for any feedback, manual or otherwise, from the user. The drug administration device 400 can have identities of trusted network(s) and/or trusted device(s) stored in the memory 97, e.g., in a look-up table. If one of the values in the look-up table matches a signature of the requesting network 420 or external device 410, then the update request can be confirmed as authorized and no manual authorization will be required. The method can thus include confirming whether the update request is authorized by confirming whether the external device 410 or the network 410 that sent the update request to the device 400 is an acceptable external device or network as previously determined as stored in the memory 97. The drug administration device 400 can remove one or more of the trusted network(s) and/or device(s) from the memory 97 either as a result of a manual input or based on inactivity from the trusted network(s) and/or device(s) within a certain predetermined amount of time (e.g., one day, two days, one week, etc.).

The look-up table including a list of the acceptable, or trusted, devices can be stored in the memory 97 of the drug administration device 400. The list of acceptable devices can be updated either manually on the drug administration device 400 (e.g., using the user interface thereof, etc.), automatically after a successful update request (e.g., because the requesting device has been determined to be trusted), remotely after a successful update request (e.g., because the requesting device has been determined to be trusted), or any combination of the above.

The drug administration device 400 can be configured to only update the parameter(s) according to the update request after both a first and a second authorization. The second update authorization can be confirmed on either the external device 410 or a computer system connected to the network 420. The second update authorization can be required to be acknowledged within a second predetermined time frame. This double authorization provides a higher level of security than a single authorization and allows a healthcare professional, for example, to confirm transfer of a new drug treatment both on the device 400 and on the network 420. That is, the method for assessing whether to update the parameter(s) of the drug administration device 400 can include requiring a second update authorization to be confirmed on either the external device 410 or a computer system connected to the network 420 within a second predetermined time frame. This ensures that, during a remote update, a doctor, or another medical professional, can also confirm the drug treatment as a second stage of verification.

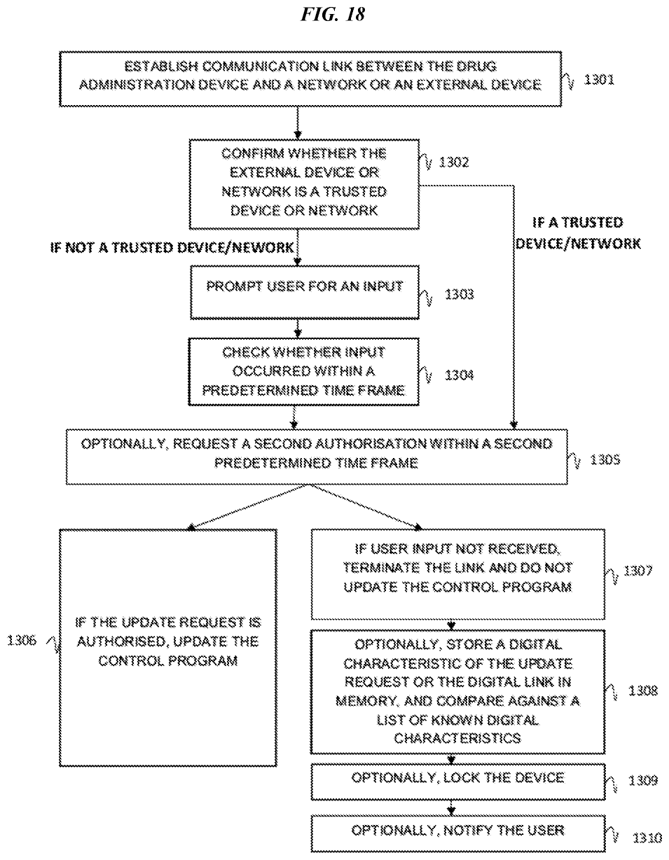

One embodiment of a method updating the of the drug administration device 400 having a security protocol is shown in FIG. 18. An update request is received 1301 by the drug administration device 400. The drug administration device 400 confirms 1302 whether the external device 410 or the network 420 that sent the update request is a trusted device or network and thereby confirming whether the update request is authorized. If the external device 410 or the network 420 that sent the update request is not a trusted device or network, the user is prompted 1303 for an input. Once the user is prompted 1303, the drug administration device 400 checks 1304 whether a user input occurred within a predetermined time frame. Optionally, the drug administration device 400 can request 1305 a second authorization within a second predetermined time frame. If the check 1304 determines that the input is received within the predetermined time frame (and if, requested 1305, that the second input is received within the second predetermined time frame), the update request is considered to be authorized and, the device 400 is updated 1306 in accordance with the update request. Alternatively, if the user input is not received (or if, requested 1305, that the second input is not received within the second predetermined time frame, the update request is considered to not be authorized, and the link 430 is terminated and the device 400 is not updated 1307 as requested in the update request. Optionally, in the event that the device 400 is not updated in accordance with the update request, a digital characteristic of the update request or the communication link is stored 1308 in the memory and compared against a list of known digital characteristics, the device 400 is locked 1309, and/or the user is notified 1310.

As an example, a user can initiate an update of the drug administration device 400 by providing an input to the device 400, such as by pressing an appropriate button on the drug administration device 400. Once the input is provided, the drug administration device 400 can initiate a wireless communication with the user's smartphone as the external device 410, which may query the network 420 for any available updates. If there are any updates available, the smartphone will download the updated parameter(s) and send an update request to the drug administration device 400. If the smartphone 410 has not previously been authorized by the drug administration device 400, the drug administration device 400 can indicate to the user that the device 400 is trying to update parameter(s), and the user can manually authorize directly on the device 400, e.g., via the user interface thereof, by pressing a button thereon, etc. Once the manual authorization is received, the processor 96 can parse out the parameter(s) in the received update request message and send data to the corresponding locations in memory 97 to override the previous parameter(s). Once finished, the processor 96 can then query the locations in the memory 97 to ensure the parameter(s) were properly updated and report this to the smartphone 410 to notify the user of a successful update.

### Intercommunication of Multiple Administration Devices

It is sometimes required to deliver more than one type of drug or medication to a patient. However, there are risks associated with delivering multiple drugs to a patient because of possible interactions between the drugs that may have adverse effects on the patient and/or result in less effective drug treatments. When a patient is treated in a hospital under doctor supervision, the medical professional can help to ensure that there are no adverse reactions between the different drugs and administer the different drugs appropriately. However, for drug administration devices that are operated either automatically or by the patient themselves, there is a higher risk of accidental drug interactions. Thus, extra care must be taken where multiple drugs are administered to a patient. That is, where multiple drugs are administered, adverse reactions may arise as a result of the combination between the drugs. These adverse reactions should be avoided.

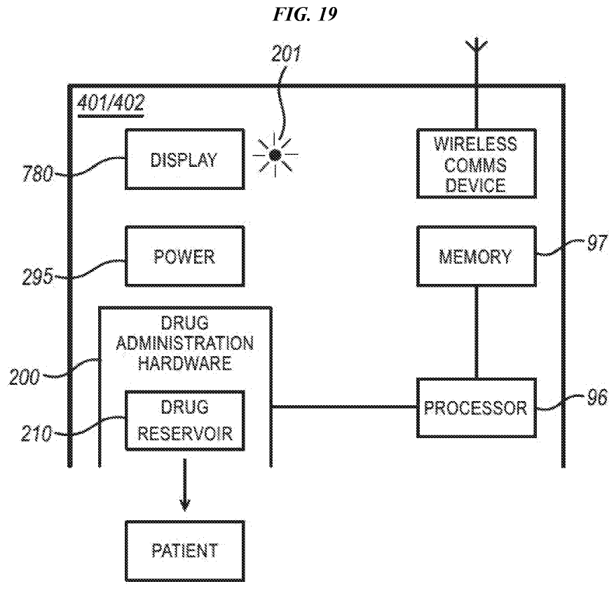

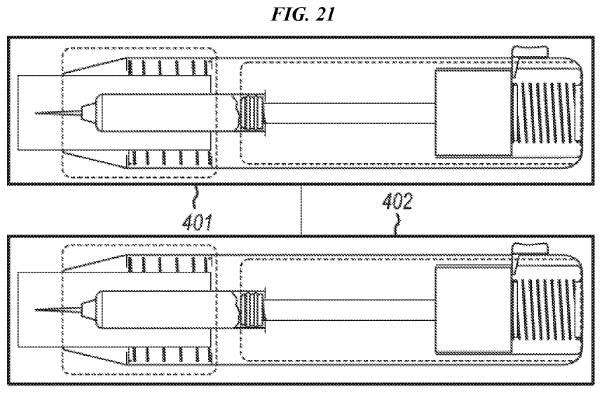

FIG. 19 illustrates an embodiment of a drug administration device 401 configured to communicate with another drug administration device 402. Such a drug administration device 401 is referred to herein as a “connected” drug administration device. Each of the drug administration devices 401, 402 can have any of the forms of drug administration devices disclosed herein, including an autoinjector (e.g., the autoinjector 100 of FIG. 1), an infusion pump (e.g., the infusion pump 200 of FIG. 2), an inhaler (e.g., the inhaler 300 of FIG. 3), or a nasal spray device. FIG. 5B illustrates an exemplary embodiment of the devices 401, 402 as drug administration device 500. FIG. 19 thus shows the drug administration devices 401, 402 as each including the processor 96 (FIG. 5B) and the memory 97 (FIG. 5B). The communications interface 99 is a wireless communications interface (comms device) in this illustrated embodiment. This wireless communication can be unidirectional (e.g., the second drug administration device 402 only broadcasts data to the first drug administration device 401 or vice versa) or bidirectional (e.g., both devices 401, 402 can send messages to each other and can respond/acknowledge to the messages). Alternatively to wireless communication, as shown in FIG. 21, the first and second drug administration devices 401, 402 can have a wired connection and be configured to communicate over the wired connection. In the embodiment of FIG. 21, there is no need for a wireless communications module, which may be preferable where security is a concern or where the two drug administration devices 401, 402 can be implemented within a single housing.

The drug administration devices 401, 402 in this illustrated embodiment of FIG. 19 also each include an indicator, such as an LED 201, which can be used to convey information to a user (e.g., power on, drug administration in process, etc.). The drug administration devices 401, 402 in this illustrated embodiment are each an infusion pump and thus are shown in FIG. 19 as including a power supply 295 (FIG. 2), a drug holder in the form of a drug reservoir 210 (FIG. 2), and the user interface 280 in the form of a display 780.

The first drug administration device 401 and the second drug administration device 402 are the same form of drug administration device in this illustrated embodiment, but the first drug administration device 401 and the second drug administration device 402 can be different forms of drug administration device.

The first drug administration device 401 is configured to administer a first drug to a patient, and the second drug administration device 402 is configured to administer a second drug to a patient. The first and second drugs can be different (e.g., for treating different illnesses), or instead, can be the same. When the first and second drugs are different, they may differ in drug type (e.g., active ingredient), concentration, and/or dosage form. The first and second drug administration devices 401, 402 can be configured to administer the same drug with different dosing parameters. For example, the first drug administration device 401 can be configured to administer the first drug as slow-release insulin, and the second drug administration device 402 can be configured to administer the second drug as a quick, larger dosing of insulin in the case of emergencies.

The processor 96 of the drug administration devices 401, 402 can each be configured to control operation of its associated drug administration device 401, 402 in accordance with a control program, which can be a dosing scheme as discussed above. By having separate control programs, each control program can be adjusted appropriately to ensure maximum flexibility in the drug administration treatment. The processor 96 can also be configured to delay operation of its associated drug administration device 401, 402 in accordance with the control program, as discussed further below.

The processor 96 of the first drug administration device 401 can be configured to avoid simultaneous operation of the first drug administration device 401 in accordance with the control program thereof (also referred to herein as a “first control program”) and operation of the second drug administration device 402 in accordance with a second control program thereof also referred to herein as a “second control program”).

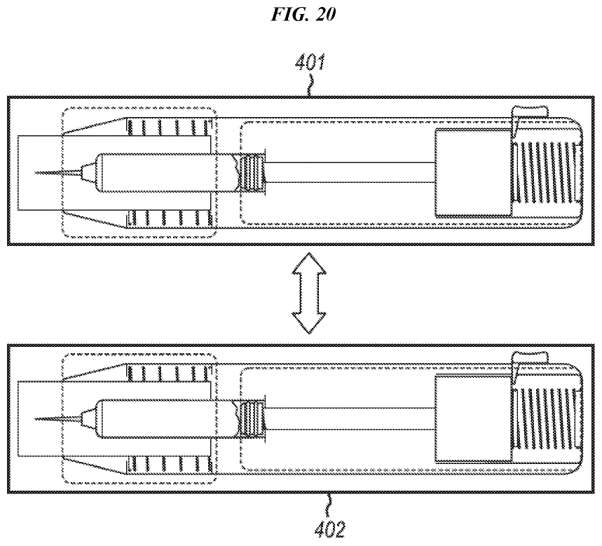

FIG. 20 illustrates an embodiment of a drug administration system in which the first drug administration device 401 is configured to communicate wirelessly with the second drug administration device 402. Wireless communication may help to prevent unnecessary physical wires extending between the devices 401, 402, which may be especially beneficial when the drug administration devices 401, 402 are located at different points on the patient's body and the drug administration devices 401, 402 need to be able to communicate to each other without interfering with patient movement. Furthermore, wireless communication may enable transmission of data to an external device which may improve the user interface and the data collection in the system as discussed herein.

The drug administration system is configured to enable adaptive, interactive dosing of the first and second drugs in a drug delivery treatment for the patient. That is, communication between the first drug administration device 401 and the second drug delivery device 402 can help to prevent errors due to interactions between the first and second devices 401, 402 or between the first and second drugs. In other words, because the processor 96 of the first drug administration device 401 is configured to adjust dosing parameter(s) of its control program (dosing scheme) in response to a communication from the second drug administration device 402, the first drug administration device 401 can adaptively deliver the first drug and thereby reducing risk of an unintended drug interaction between the first and second drugs 401, 402.

Beyond preventing adverse drug reactions, the coordination of the administration of the two drugs from the first and second devices 401, 402 may help optimize the patient therapy. As an example, the first drug may be most effective when it is administered either two hours after the second drug or at a certain time (e.g., immediately prior to the patient's sleep cycle). The intercommunication of multiple drug delivery devices 401, 402 can help ensure the optimum approach is adopted. Two drug administration devices 401, 402 are shown in FIGS. 19 and 20 (and in FIGS. 21, 24, and 25), but more than two drug administration devices each for delivering a drug to the same patient can be used similar to the two drug administration devices 401, 402 discussed herein.

The second drug administration device 402 can be configured to communicate data with details about its own administration (e.g., time initiated, duration, etc.) in the form of one or more dosing parameters of the second drug administration device's control program to the first drug administration device 401. At least one dosing parameter that is stored in the memory 97 of the first drug administration device 401 can be adjusted, e.g., by the first drug administration device's processor 96, in response to the communication received from the second drug administration device 402. In this way, the administration of the first drug from the first drug administration device 401 can be adjusted to account for information relating to the second drug administration device 402. For example, a dosing parameter relating to a timing of the next administration of the first drug can be adjusted based on a communication from the second drug administration device 402.

The processor 96 of the first drug administration device 401 can be configured to receive this data from the second device 402 and compare the received parameter(s) against data stored in a look-up table in the memory 97 of the first device 401. For example, if the second drug administration device 402 sends data indicating that its administration finished at a certain time, the processor 96 of the first drug administration device 401 (also referred to herein as a “first processor”) can be configured to determine how much time has elapsed since that certain time and compare the elapsed against a parameter that defines a predetermined acceptable elapsed time (e.g., ten minutes, fifteen minutes, thirty minutes, etc.). It may be, for example, only be acceptable for the first drug administration device 401 to administer a drug if the predetermined acceptable elapsed time has passed since the second drug administration device 402 finishes a drug administration sequence.

After comparing the communicated data to values stored in the memory 97, the first processor 96 can adjust the control program of the first drug administration device 401. That is, the first processor 96 can be configured to lower the dosage of the first drug, delay the administration of the first drug, etc.

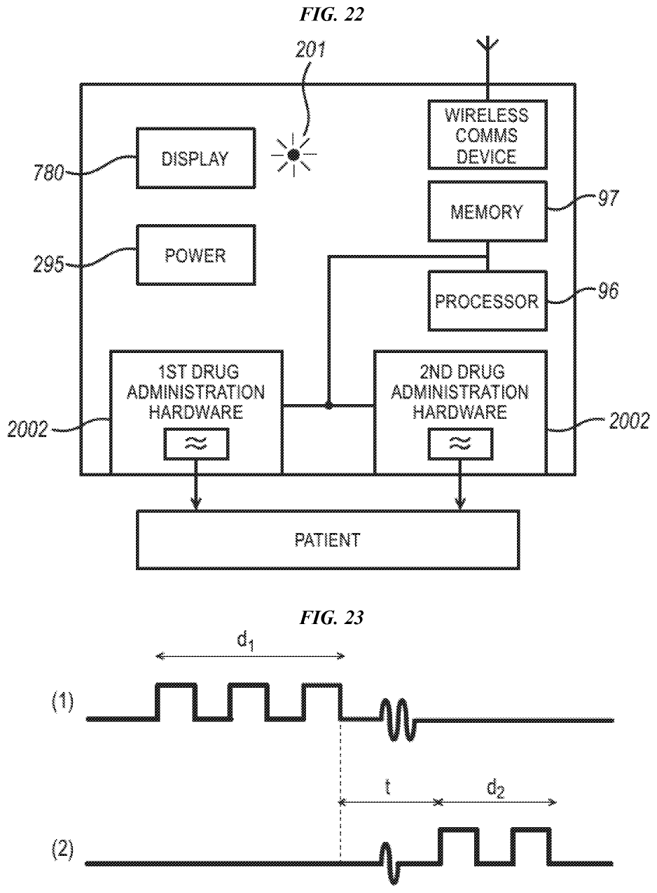

FIG. 22 illustrates an embodiment in which the first and second drug administration devices 401, 402 are contained within a single housing. In other words, both drug administration devices 401, 402 are encased together within a single mechanical mold, which may be beneficial to reduce an amount of required hardware and thereby reduce cost and size, increase the convenience of handling the two devices 401, 402 and/or minimize the number of medical devices the patient needs to manage. In this illustrated embodiment, the first and second drug administration devices 401, 402 share resources contained at least partially within the housing, including the processor 96, the memory 97, the LED 201, the wireless communications device, the display 708, and the power supply 295. In other embodiments in which the first and second drug administration devices 401, 402 are contained in the same housing, each device 401, 402 can have its components within the housing, such as its own power supply 295 and/or its own processor 96 and can be configured to communicate with the other and, possibly, other remote devices, either wirelessly or through a wired connection.

The first drug administration device 401 and the second drug administration device 402 contained within the single housing can share a component for drug delivery, e.g., use a same needle to deliver the first and second drugs, use the same spring and/or motor for injection, etc.

FIG. 23 illustrates an embodiment of a timing diagram of two sample control programs for the first and second drug administration devices 401, 402 of any of FIGS. 19-22. Each drug administration device 401, 402 can be configured to administer a drug according to its own control program, as discussed above. Each respective control program defines a method of how the drug is administered using dosing parameters such as an amount of the drug delivered, a frequency of the drug administration, and a duration of the drug administration. With these dosing parameters, the treatment plan can be fully adjusted for the specific patient, based on known best practices for drug administration. As shown in FIG. 23, the control program of the first drug administration device 401 can control administration of the first drug in a series of consecutive administrations in a first control program sequence d₁. Alternatively, the control program of the first drug administration device 401 can control administration of the first drug during a single interval. As also shown in FIG. 23, initiation of the control program of the second drug administration device 402 in a series of consecutive administrations in a second control program sequence d₂ can be delayed until a certain time t has elapsed after the end of the first control program sequence d₁. The control program of the second drug administration device 402 can instead control administration of the second drug during a single interval.

As discussed herein, the dosing parameters of the first and second drug administration device's control programs can be updated by the processor 96 as discussed herein, which may optimize the patient's drug treatment and/or avoid any unnecessary drug interactions between the first and second drugs. the memory 97 for the devices 401, 402 can store default parameters for the first and second drug administration device's control programs, which can be set either locally on the device or remotely by a secure server. The default parameters can be default parameters which, in the event of a communication failure, each drug administration device 401, 402 would be configured to use. This use of default parameters may help ensure safety of the drug administration system in event of communication error. For example, if communication fails between any part of the drug administration system, e.g., between the first and second drug administration devices 401, 402, between either the first drug administration device 401 or the second drug administration device 402 and an external device, or between a physiological sensor and a drug administration device 401, 402 or an external device, the drug administration system can be configured such that the control program of at least one of the drug administration devices 401, 402 is reset to default parameters.

One or more of the dosing parameters of the first drug administration device 401 can be adjusted in response to a communication from the second drug administration device 402. Likewise, one or more dosing parameters of the second drug administration device 402 can be adjusted in response to a communication from the first drug administration device 401. The communication from the first drug administration device 401 or second drug administration device 402 can include a message including a confirmation of administration of a drug. This confirmation can include a time stamp.

A drug administration system including the first and second drug administration devices 401, 402 of any of FIGS. 19-22 can also include an external device configured to communicate with both the first drug administration device 401 and the second drug administration device 402. The system including an external device in addition to the first and second drug administration devices 401, 402 may be beneficial because an external device may have improved means for tracking and/or displaying any data regarding drug administration, and any hardware already present in the external device (e.g., a clock, a processor, a user interface, an alarm system, etc.) can be utilized without requiring more hardware to be introduced into either or both drug administration devices 401, 402.

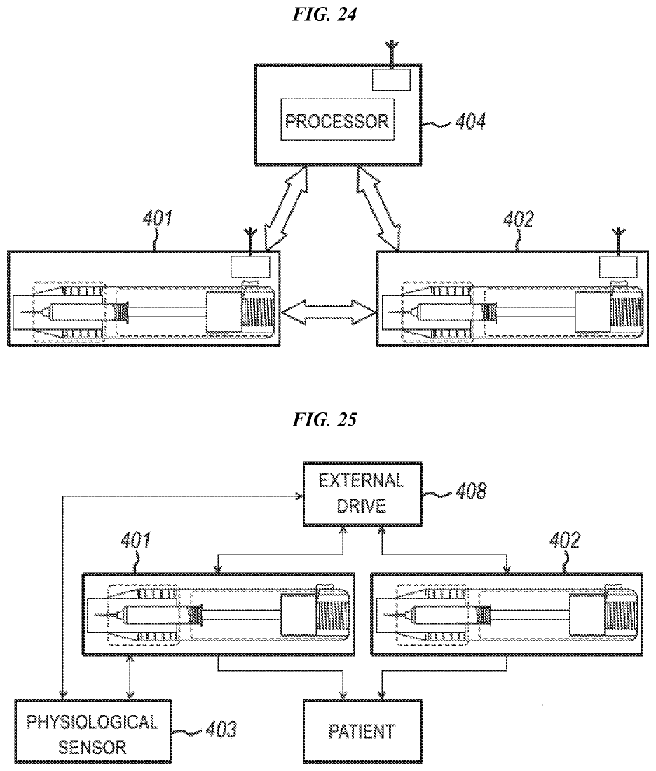

FIG. 24 illustrates an embodiment of a drug administration system in which the first and second drug administration devices 401, 402 are each configured to communicate with an external device 404 configured to communicate with the first and second drug administration devices 401, 402. The external device 404 is external to the drug administration devices 401, 402 and is similar to the external device 900 discussed above, e.g., can include a computer system with wireless communication capabilities and including a processor, a user interface, a display, etc. The display of the external device 404 can be configured to graphically show any of the parameters of the dosing schemes of the first and second devices 401, 402, such as when, and how often, each of the first and second drugs are administered on a day.

The external device 404 can be configured to provide coordination of drug administration between the first and second drug administration devices 401, 402. In other words, the external device 404 can be configured to initiate operation of each of the drug administration devices 401, 402, in accordance with their respective control programs that can be stored in the memory 97 of the associated drug administration device or in the memory of the external device 404. The external device 404 can also be configured to confirm whether the patient is in the correct state for administering the first drug from the first drug administration device 401 by receiving a communication from the second drug administration device 402 or vice versa for the second drug. One embodiment of the external device 404 configured to provide coordination of drug administration between the first and second drug administration devices 401, 402 is the smartphone 500 of FIG. 10A, which can be configured to wirelessly transmit first and second administration signals to the first and second drug administration devices 401, 402, respectively, to cause drug administration to begin in response to receipt of the administration signal.

One of the first drug administration device 401 and the external device 404 can act as a primary controller configured to operate the first drug administration device 401 in accordance with the first control program and operate the second drug administration device 402 in accordance with the second control program. In other words, the primary controller acts as a single controller in communication with both drug administration devices 401, 402. The primary controller may minimize an amount of computing hardware required within each drug administration device 401, 402 and thus reduce cost and/or size.

The primary controller is configured to coordinate, or control, timing of drug administration from the first and second drug administration devices 401, 402, order of drug administration from the first and second drug administration devices 401, 402, and how the first and second drugs are delivered to the patient from the first and second drug administration devices 401, 402.

The primary controller can include a user interface, which may allow a user to manually change one or more dosing parameters of the first and/or second control program. Allowing manual change may be beneficial because such a drug administration system can be manually set to specific parameters at different times to update or change the drug treatment. For example, a medical professional may wish to manually override the drug dosage to taper off painkiller medication near the end of a drug treatment or to manually increase the allowable number of dose administrations from the drug administration device to account for a prior mis-administration. This manual change can be used to set up default parameters or to set up timing administration of the first and second drugs. The manual change can allow the user to override drug administration of one or both of the drug administration devices 401, 402 by providing feedback to the primary controller that the primary controller implements as needed by adjusting the first and/or second control program in accordance with the manual input.

The external device 404 can store parameters of the control programs (dosing schemes) of the first and second drug administration devices 401, 402 in its own electronic memory, e.g., dosing parameters of the first control program associated with the first drug administration device 401 and dosing parameters of the second control program associated with the second drug administration device 402.

The external device 404 can be configured to communicate with a network and thus a computer system configured to communicate over the network as discussed above. In this way, the external device 404 can be configured to report data related to the drug treatment using the first and second drug administration devices 401, 402, over a wireless connection, to the computer system. The external device 404 can also be configured to report communication faults to the first and second drug administration devices 401, 402, which may be in the form of an alert to the user via a device indicator, such as the LED 201, audible indication, or a message shown on the display 780. Communication faults can also be reported to the computer system. A communication fault, such as a failure to communicate between the drug administration devices 401, 402 can be configured to trigger a phone call to a medical professional or to an information technology (IT) service to assist the patient.

In other embodiments, as discussed above, the first drug administration device 401 can be configured to receive communications directly from the second drug administration device 402 without the external device 404 acting as an intermediary. It may be beneficial for drug administration devices 401, 402 to communicate directly, without any intermediary, as such a drug administration system may be less prone to communication failures and may have fewer vulnerabilities to communication failures or to hacking attempts, as such drug administration device 401, 402 may not be responsive to signals external to the system, e.g., to signals from the external device 404. It may, however, be beneficial to use the external device 404 as an intermediary for data communication between the first and second drug administration devices 401, 402 because such a system may improve flexibility because the drug administration system can rely on the hardware already present in the external device 404 to accomplish the processing features and/or because the external device 404 can act as a hub for receiving information from sources in addition to the first and second drug administration devices 401, 402. Further, an external device 404 in the drug administration system may improve the ability to notify the patient of any irregularities, display information about the drug treatment plan, and communicate that information to another external network. In this way, each drug administration device 401, 402 can be simplified, and the external device 404 can have improved reporting, analysis, and remote operation capabilities. For example, the external device 404 can be the patient's smartphone, and the patient can utilize their smartphone as both a remote control for the drug administration devices 401, 402 and a data collector for graphically displaying features of the drug treatment.

The first drug administration device 401 can be configured to receive communication directly from the second drug administration device 402 (e.g., with no external device 404 acting as an intermediary) and can also be configured to receive a communication indirectly from the second drug administration device 402 via the external device 404. In other words, the second drug administration device 402 may be configured to communicate data to the external device 404, which is then configured to reroute the communication, or send a new, corresponding message, to the first drug administration device 401.

Interaction parameters can be stored in the memory 97 of either one or both of the drug administration devices 401, 402 and/or in the external device 404 in embodiments including the external device. In other words, it may be known that certain drugs have adverse effects when administered simultaneously. Thus, the interaction parameters can define a look-up table including any number of dosing requirements as a result of the specific drugs being administered, e.g., the first and second drugs being administered from the first and second drug administration devices 401, 402. The interaction parameters are dosing parameters that define dosing requirements based on the combination of the first drug and the second drug. By storing and using interaction parameters, the drug administration system may more effectively ensure that severe drug interactions are avoided than if the interaction parameters were not used. As an example, if the second drug cannot be administered within a certain amount of time (e.g., ten minutes, one hour, etc.) of administration of the first drug, an interaction parameter can be stored in the memory 97 and accessed by the processor 96 indicating the time delay for the first drug. Thus, once the first drug is administered, the system can be configured to prevent the second drug from being administered for a time at least as long as specified by the interaction parameter indicating the time delay. In this way, the processor 96 can be configured to delay administration of the second drug and/or adjust a dosage of the second drug based on the administration of the first drug. This delay may ensure that administration of the first and second drugs does not occur simultaneously or that drug administration of the second drug does not happen within the predetermined time after administration of the first drug starts or ends as stored in the interaction parameter(s). The dosage adjustment may help reduce adverse drug interactions between the first and second drugs or help prevent the second drug administration device 402 from delivering any of the second drug until the first drug administration device 401 has completed delivering the first drug. Preventing the second drug from being delivered until after the first drug has been completely delivered may help reduce side effects that could be caused by interaction between the first drug and second drug and/or may help reduce chance of drug abuse by preventing the second drug from being delivered until the first drug has been delivered as prescribed.

Other examples of interaction parameters include information relating to adverse reactions between different drugs, e.g., severity of interaction, type of interaction, etc. As an example, there can be an interaction parameter stored in the memory which indicates that a heart medication and insulin should not be administered simultaneously or subsequent to one another within a certain time period.

Either one or both of the first or second drug administration devices 401, 402 (and/or the external device 404 in embodiments including the external device 404) can include a clock or a suitable timer. The clock or other suitable timer is configured to be in communication with the processor 96 of the respective drug administration device or external device 404 that includes the clock or other suitable timer. The clock or other suitable timer can help ensure that the first and second drugs are administered on time at specific times during the day as specified in the appropriate control program and/or that proper time delay is observed between deliveries of the first and second drugs.

The second drug administration device 402 (or the external device 404 acting as a primary controller) can be configured to transmit information to the first drug administration device 401 including, but not limited to, a time at which the second control program began, a length of the second control program, and a time at which the second control program finished. The information transmitted to the first drug administration device 401 can relate to the dosing parameters associated with the second drug administration device 402. The information transmitted to the first drug administration device 401 can include an indication of enacted dosing parameters for the second drug administration device 402, e.g., an indication that a certain volume of dose of the second drug was administered at a certain time. Any combination of this data can be utilized by the processor 96 of the first drug administration device 401 (or the external device 404 acting as primary controller) to delay operation of the first drug administration device 401, e.g., delay execution of the first control program to avoid adverse drug interactions.

Similarly, the first drug administration device 401 (or the external device 404 acting as a primary controller) can be configured to transmit information to the second drug administration device 402, including, but not limited to, a time at which the first control program began, a length of the first control program, and a time at which the first control program finished. in this way, the second control program can be adjusted, e.g., by the processor 96 of the second drug administration device 402, based on administration of the first drug by the first drug administration device 401 in the same way that the first control program can be adjusted based on administration of the second drug by the second drug administration device 402.

The primary controller in the system is configured to gather data from both the first and second drug administration devices 401, 402 and adjust both the first and the second control programs to optimize the patient's treatment. As discussed above, either one of the drug administration devices 401, 402 (or the external device 404 if present) can be configured to operate as the primary controller in the system.

After administering a drug, the corresponding drug administration device 401, 402 can be configured to report whether the administration of the drug was successful to the primary controller. Any faults detected by the drug administration device 401, 402 as part of the drug administration, e.g., power supply issues, incomplete drug administration, etc., can be communicated to the primary controller and can be utilized by the primary controller to prevent further drug administration or notify a user or a medical professional (via any of the first device 401, second device 402, external device, or other device) to take any desired corrective action to address the detected fault(s).

The processor of the primary controller can be configured to prioritize administration from one of the drug administration devices 401, 402. This prioritization may be beneficial where of the first and second drugs is life-critical, but the other of the first and second drugs on the patient's treatment plan is non-essential (e.g., pain-killers, etc.). The processor of the primary controller can be configured to adaptively adjust the operation of the first and second drug administration devices 401, 402, e.g., by adjusting the first control program and/or second control program, such that the essential, life-critical drug is always administered, either always at the same time or at the expense of the administration of the non-essential drug. For another example, the non-essential drug can only be administered after the life-critical drug has been administered.

FIG. 25 illustrates an embodiment of a drug administration system including the first and second drug administration devices 401, 402, an external device 408 (also referred to herein as an “external drive”), and a physiological sensor 403 configured to sense a user physiological parameter of a patient. The external device 408 is external to the drug administration devices 401, 402 and the sensor 403 and is similar to the external device 900 discussed above.

The physiological sensor 403 can be wired or wireless and can be connected (wired or wirelessly) to either or both of the first and second drug administration devices 401, 402 and/or to the external device 408. Using the physiological sensor 403 in the drug administration system may help to optimize the administration of multiple drug administration devices 401, 402 by not relying on default time parameters alone. Thus, for especially safety critical applications, or where the known interactions between the first and second drugs is especially dangerous, it may be more suited to specifically monitor one or more physiological parameters of the patient to ensure that a certain parameter (e.g., hormone level, pH, blood pressure or other blood characteristic, drug concentration, pupil dilation, etc.) is acceptable before administering the first drug and/or the second drug. For example, the physiological sensor 403 can be configured to measure a concentration of the first drug in the body of the patient, and report this information back to the primary controller (either one of the drug administration devices 401, 402 or the external device 408), which can instruct the second drug administration device 402 to prevent delivery of the second drug until the concentration of the first drug falls below an acceptable level as sensed by the physiological parameter and verified by the primary controller to be below a predetermined acceptable threshold value or noted as acceptable in a look-up table of parameter values.

At least one dosing parameter of the first control program and/or second control program can be adjusted, e.g., by the processor of the primary controller, in response to the sensed body parameter, which may allow a variety of biometrics to be monitored to optimize drug treatment. Taking the physiological parameter into account may be more effective than solely relying on time elapsed between administrations of the first and second drugs by ensuring a safe condition for drug administration based on the physiology of the patient. Further, interactions between medications can also occur between the first drug and/or the second drug and other drugs that patient may receive, such as ibuprofen and paracetamol. To avoid interaction with other drugs not administered by one of the drug administration devices 401, 402, the physiological sensors 403 may be utilized to prevent interaction similar to that discussed above with respect to the first and second drugs.

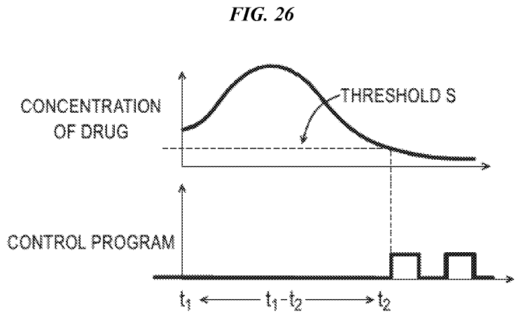

FIG. 26 illustrates an embodiment of a physiological parameter measured, drug concentration in this illustrated embodiment, as an indication that the first drug administered by the first drug administration device 401 should be not delivered given the measured parameter value, or that the delivery thereof should be adjusted given the measured parameter value. If the first drug administration device 401 attempts to administer the first drug at time t₁ but the measured physiological parameter is above a threshold S, operation of the first drug administration device can be delayed (e.g., by the primary controller changing the first control program, namely a dosing parameter thereof for delivery time or frequency) by a time t₂−t₁ until the measured physiological parameter is below a predetermined threshold S, at which point it is deemed safe to administer a drug to the patient. As shown in FIG. 26, the first control program starts at time t₂, which corresponds to the time at which the measured physiological parameter is below the threshold S. The predetermined threshold S corresponds to a condition in which administration of the first drug is assumed to be safe, with respect to the drug having its concentration measured, without any negative interaction with either the patient or the previously administered drug. The second drug administration device 402 can have its control program similarly adjusted by the primary controller based on the measured physiological parameter.

When the drug administration system includes the physiological sensor 403, the drug administration system can be configured to adjust dosing parameters in response to both interaction parameters as discussed above and physiological parameters as discussed above. In this way, the drug administration system can be configured such that the first drug only be delivered after a predetermined number of minutes after administration of the second drug (or vice versa) and/or when a measured physiological parameter is above/below a certain predetermined threshold.

The physiological sensor 403 is configured to sense one or more physiological parameters. The physiological sensor 403 can be a single sensor or can be a plurality of sensors. Each of the plurality of sensors can be configured to measure a different parameter, which all may be relevant to the determination of optimizing drug treatments.

All of the devices and systems disclosed herein can be designed to be disposed of after a single use, or they can be designed to be used multiple times. In either case, however, the devices can be reconditioned for reuse after at least one use. Reconditioning can include any combination of the steps of disassembly of the devices, followed by cleaning or replacement of particular pieces, and subsequent reassembly. In particular, the devices can be disassembled, and any number of the particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

It can be preferred that devices disclosed herein be sterilized before use. This can be done by any number of ways known to those skilled in the art including beta or gamma radiation, ethylene oxide, steam, and a liquid bath (e.g., cold soak). An exemplary embodiment of sterilizing a device including internal circuitry is described in more detail in U.S. Pat. Pub. No. 2009/0202387 published Aug. 13, 2009 and entitled “System And Method Of Sterilizing An Implantable Medical Device.” It is preferred that device, if implanted, is hermetically sealed. This can be done by any number of ways known to those skilled in the art.

The present disclosure has been described above by way of example only within the context of the overall disclosure provided herein. It will be appreciated that modifications within the spirit and scope of the claims may be made without departing from the overall scope of the present disclosure.

What is claimed is:

1. A computer-implemented method of generating a data packet comprising drug administration data and ancillary data, wherein the drug administration data has a first data format and relates to parameters of a drug administration device, the method comprising:
establishing communication between the drug administration device and at least one of a network and an external device;
receiving the ancillary data from the at least one of the network and the external device at the drug administration device;
generating the data packet at the drug administration device by:
filtering the ancillary data such that ancillary data relating to a user parameter remains;
converting at least the ancillary data relating to the user parameter into the first data format; and
combining the drug administration data and the ancillary data relating to the user parameter.

2. The method of claim 1, further comprising the drug administration device adjusting at least one operational parameter of the drug administration device based on the data packet.

3. The method of claim 2, wherein adjusting the at least one operational parameter comprises:
comparing a data packet value to a reference data packet value; and
adjusting the at least one operational parameter of the drug administration device based on the comparison.

4. The method of claim 2, wherein adjusting the at least one operational parameter comprises:
comparing a plurality of data packet values to a plurality of reference data packet values; and
adjusting the at least one operational parameter of the drug administration device based on the comparisons.

5. The method of claim 3 or claim 4, wherein the reference data packet value relates to at least one of:
a physiological condition of a user;
a psychological condition of a user; and
an environmental conditional of a user.

6. The method of any one of claims 2 to 5, wherein the at least one operational parameter relates to at least one of:
a dosage amount;
a dosage frequency;
a maximum number of doses deliverable from the drug administration device; and
a dosage time.

7. The method of any preceding claim, further comprising communicating the data packet to a central computer system connected to the network.

8. The method of claim 7, further comprising communicating an administration signal from the central computer system to the drug administration device upon receipt of the data packet, the drug administration device configured to permit administration of a drug only upon receipt of the administration signal.

9. The method of claim 7 or claim 8, further comprising the central computer system determining, based on the data packet, that at least one of a drug refill, emergency services, a user reminder prompt, and a variation in at least one of the parameters of the drug administration device is required.

10. The method of any preceding claim, further comprising communicating the data packet to a display device connected to the network.

11. The method of any preceding claim, further comprising the drug administration device determining, based on the data packet, that a drug refill or emergency services are required and communicating a status signal of the drug administration device to the central computer system.

12. The method of any preceding claim, wherein establishing communication comprises the drug administration device scanning for available networks and external devices and establishing communication with the available networks and/or external devices.

13. The method of any preceding claim, wherein establishing communication comprises establishing wireless communication between the drug administration device and the at least one of the network and the external device.

14. The method of any preceding claim, wherein the ancillary data relating to the user parameter comprises at least one of:
a location of a user;
an environmental condition of a user;
a psychological condition of a user and
a physiological condition of a user.

15. The method of claim 14, wherein the location of the user, the physiological condition of the user, and/or the environmental condition of the user are measured using at least one sensor of the external device.

16. The method of claim 14 or claim 15, wherein the location of the user, the physiological condition of the user, the psychological condition of the user, and/or the environmental condition of the user are measured continuously, at regular intervals, when requested by a user, or when requested by the drug administration device.

17. The method of any preceding claim, wherein the parameters of the drug administration device comprise at least one of:
drug administration history;
an amount of drug remaining in the drug administration device;
drug composition data; and
power consumption data.

18. The method of any preceding claim, wherein the drug administration data and the ancillary data each comprise associated time values.

19. The method of any preceding claim, wherein the external device is configured to record and/or influence a physiological condition of a user, and the external device comprises one or more of: a user stress testing device; a user exercise device; a fitness wearable; a CPAP machine; a blood glucose monitor; and a heart rate monitor.

20. The method of any preceding claim, wherein the drug administration device is configured to deliver a drug, and the drug comprises at least one of infliximab, golimumab, ustekinumab, daratumumab, guselkumab, epoetin alfa, risperidone, esketamine, ketamine, and paliperidone palmitate.

21. A drug administration system, comprising:
at least one of a network and an external device;
a drug administration device comprising:
a housing that accommodates a drug holder and a dispensing mechanism;
a communications interface configured to:
establish communication between the drug administration device and the at least one of the network and the external device;
receive ancillary data from the at least one of the network and the external device; and
a processor configured to:
receive drug administration data having a first data format and relating to parameters of the drug administration device;
filter the ancillary data such that ancillary data relating to a user parameter remains;
convert at least the ancillary data relating to the user parameter into the first data format; and
combine the drug administration data and the ancillary data relating to the user parameter to thereby generate a data packet.

22. The system of claim 21, wherein the processor is further configured to adjust at least one operational parameter of the drug administration device based on the data packet.

23. The system of claim 22, wherein the processor is further configured to compare a data packet value to a reference data packet value and adjust the at least one operational parameter of the drug administration device based on the comparison to thereby adjust the at least one operation parameter.

24. The system of claim 22, wherein the processor is further configured to compare a plurality of data packet values to a plurality of reference data packet values and adjust the at least one operational parameter of the drug administration device based on the comparisons to thereby adjust the at least one operation parameter.

25. The system of any one of claims 21 to 24, wherein the communications interface is further configured to communicate the data packet to a central computer system connected to the network.

26. The system of claim 25, wherein the central computer system is configured to communicate an administration signal to the drug administration device upon receipt of the data packet, and wherein the processor is configured to permit administration of a drug only upon receipt of the administration signal at the communications interface.

27. The system of claim 25 or claim 26, wherein the central computer system is configured to determine, based on the data packet, that at least one of a drug refill, emergency services, a user reminder prompt, and a variation in at least one of the parameters of the drug administration device is required.

28. The system of any one of claims 21 to 27, wherein the communications interface is further configured to communicate the data packet to a display device connected to the network.

29. The system of any one of claims 21 to 28, wherein the processor is configured to determine, based on the data packet, that a drug refill or emergency services are required and to communicate a status signal of the drug administration device to a central computer system connected to the network.

30. The system of any one of claims 21 to 29, wherein the communications interface is configured to scan for available networks and external devices and to establish communication with the available networks and/or external devices.

31. The system of any one of claims 21 to 30, wherein the communications interface is configured to establish wireless communication between the drug administration device and the at least one of a network and the external device.

32. The system of any one of claims 21 to 31, wherein the external device comprises a sensor configured to measure a location of the user, a physiological condition of the user, and/or an environmental condition of the user.

33. The system of claim 32, wherein the sensor is configured to measure the location of the user, the physiological condition of the user, and/or the environmental condition of the user continuously, at regular intervals, when requested by a user, or when requested by the drug administration device.

34. The system of any one of claims 21 to 33, wherein the drug administration data and the ancillary data each comprise associated time values.

35. The system of any one of claims 21 to 34, wherein the external device is configured to record and/or influence a physiological condition of a user, and the external device comprises one or more of: a user stress testing device; a user exercise device; a fitness wearable; a CPAP machine; a blood glucose monitor; and a heart rate monitor.

36. The system of any preceding claim, wherein the drug holder is configured to hold a drug, the dispensing mechanism is configured to dispense the drug, and the drug comprises at least one of infliximab, golimumab, ustekinumab, daratumumab, guselkumab, epoetin alfa, risperidone, esketamine, ketamine, and paliperidone palmitate.

37. A drug administration device comprising:
a housing that accommodates a drug holder and a dispensing mechanism;
a communications interface configured to:
establish communication between the drug administration device and at least one of a network and an external device; and
receive ancillary data from the at least one of the network and the external device; and
a processor configured to:
receive drug administration data having a first data format and relating to parameters of the drug administration device;
filter the ancillary data such that ancillary data relating to a user parameter remains;
convert at least the ancillary data relating to the user parameter into the first data format; and
combine the drug administration data and the ancillary data relating to the user parameter to thereby generate a data packet.

38. The device of claim 37, wherein the processor is further configured to adjust at least one operational parameter of the drug administration device based on the data packet.

39. The device of claim 38, wherein the processor is further configured to compare a data packet value to a reference data packet value and adjust the at least one operational parameter of the drug administration device based on the comparison to thereby adjust the at least one operation parameter.

40. The device of claim 38, wherein the processor is further configured to compare a plurality of data packet values to a plurality of reference data packet values and adjust the at least one operational parameter of the drug administration device based on the comparisons to thereby adjust the at least one operation parameter.

41. The device of any one of claims 37 to 40, wherein the communications interface is further configured to communicate the data packet to a central computer system connected to the network.

42. The device of claim 41, wherein the processor is configured to permit administration of a drug only upon receipt of an administration signal at the communications interface from the central computer system.

43. The device of any one of claims 37 to 41, wherein the communications interface is further configured to communicate the data packet to a display device connected to the network.

44. The device of any one of claims 37 to 43, wherein the processor is configured to determine, based on the data packet, that a drug refill or emergency services are required and to communicate a status signal of the drug administration device to a central computer system connected to the network.

45. The device of any one of claims 37 to 44, wherein the communications interface is configured to establish wireless communication between the drug administration device and the at least one of a network and the external device.

46. The device of any one of claims 37 to 45, wherein the drug administration data and the ancillary data each comprise associated time values.

47. The device of any preceding claim, wherein the drug holder is configured to hold a drug, the dispensing mechanism is configured to dispense the drug, and the drug comprises at least one of infliximab, golimumab, ustekinumab, daratumumab, guselkumab, epoetin alfa, risperidone, esketamine, ketamine, and paliperidone palmitate.

48. A method of adjusting at least one operational parameter of a drug administration device, the method comprising:
scanning for at least one external device with the drug administration device;
establishing communication between the drug administration device and the at least one external device;
receiving user physiological data from the at least one external device at the drug administration device; and
adjusting at least one operational parameter of the drug administration device based on the user physiological data.

49. The method of claim 48, wherein the user physiological data comprises a first user physiological parameter and a second user physiological parameter different from the first user physiological parameter, and wherein adjusting the at least one operational parameter comprises:
adjusting the at least one operational parameter based on the first user physiological parameter; or
adjusting the at least one operational parameter based on the second user physiological parameter.

50. The method of claim 49, further comprising adjusting the at least one operational parameter based on the first user physiological parameter if the second user physiological parameter is within a first range, and adjusting the at least one operational parameter based on the second user physiological parameter if the second user physiological parameter is within a second range.

51. The method of claim 50, wherein a limit of the first range is equal to a threshold value, and the threshold value is a boundary of the second range.

52. The method of claim 50 or claim 51, when the second user physiological parameter is within the second range, further comprising:
determining an acceptable range of adjustment values of the at least one operational parameter based on the first user physiological parameter;
determining a first adjustment value of the at least one operational parameter based on the second user physiological parameter; and
adjusting the at least one operational parameter to the first adjustment value if the first adjustment value is within the acceptable range of adjustment values, and adjusting the at least one operational parameter based on the first user physiological parameter if the first adjustment value is outside the acceptable range of adjustment values.

53. The method of claim 52, wherein adjusting the at least one operational parameter when the first adjustment value is outside the acceptable range of adjustment values further comprises adjusting the at least one operational parameter based on the first user physiological parameter and the second user physiological parameter.

54. The method of any one of claims 49 to 51, wherein adjusting the at least one operational parameter further comprises adjusting the at least one operational parameter based on the first user physiological parameter and the second user physiological parameter.

55. The method of any one of claims 49 to 54, wherein the first user physiological parameter and the second user physiological parameters are received from the same external device or from separate external devices.

56. The method of any one of claims 48 to 55, further comprising identifying a user physiological parameter that the user physiological data relates to.

57. The method of claim 56, further comprising prompting a user to confirm that the at least one operation parameter should be adjusted based on the identified user physiological parameter.

58. The method of any one of claims 48 to 57, wherein communication is established manually or automatically.

59. The method of any one of claims 48 to 58, wherein the at least one external device comprises a plurality of external devices.

60. The method of any one of claims 48 to 59, wherein the at least one operational parameter relates to at least one of:
a dosage amount;
a dosage frequency; and
a dosage time.

61. The method of any one of claims 49 to 60, wherein the user physiological data relates to at least one of:
blood concentration of dispensed drug;
blood glucose level; and
heart rate.

62. The method of any one of claims 48 to 61, wherein the at least one external device is an implanted device.

63. The method of any one of claims 48 to 56, wherein the drug administration device is configured to deliver a drug, and the drug comprises at least one of infliximab, golimumab, ustekinumab, daratumumab, guselkumab, epoetin alfa, risperidone, esketamine, ketamine, and paliperidone palmitate.

64. A drug administration system, comprising:
at least one external device comprising a sensor configured to measure user physiological data:
a drug administration device comprising:
a housing that accommodates a drug holder and a dispensing mechanism;
a communications interface configured to:
scan for the at least one external device with which to establish communication;
establish communication between the drug administration device and the at least one external device; and
receive the user physiological data from the at least one external device; and
a processor in communication with the communications interface, the processor configured to adjust at least one operational parameter of the drug administration device based on the user physiological data.

65. The system of claim 64, wherein the user physiological data comprises a first user physiological parameter and a second user physiological parameter different from the first user physiological parameter, and wherein the processor is further configured to:
adjust the at least one operational parameter based on the first user physiological parameter; and
adjust the at least one operational parameter based on the second user physiological parameter.

66. The system of claim 65, wherein the processor is further configured to adjust the at least one operational parameter based on the first user physiological parameter if the second user physiological parameter is within a first range, and to adjust the at least one operational parameter based on the second user physiological parameter if the second user physiological parameter is within a second range.

67. The system of claim 66, wherein a limit of the first range is equal to a threshold value, and the threshold value is a boundary of the second range.

68. The system of claim 66 or claim 67, wherein when the second user physiological parameter is within the second range the processor is further configured to:
determine an acceptable range of adjustment values of the at least one operational parameter based on the first user physiological parameter;
determine a first adjustment value of the at least one operational parameter based on the second user physiological parameter; and
adjust the at least one operational parameter to the first adjustment value if the first adjustment value is within the acceptable range of adjustment values, and adjust the at least one operational parameter based on the first user physiological parameter if the first adjustment value is outside the acceptable range of adjustment values.

69. The system of claim 68, wherein the processor is further configured to adjust the at least one operational parameter based on the first user physiological parameter and the second user physiological parameter when the first adjustment value is outside the acceptable range of adjustment values.

70. The system of any one of claims 65 to 67, wherein the processor is further configured to adjust the at least one operational parameter based on the first user physiological parameter and the second user physiological parameter.

71. The system of any one of claims 65 to 70, wherein a first sensor is configured to measure the first user physiological parameter and a second sensor is configured to measure the second user physiological parameter, and wherein either:
the at least one external device comprises a first external device comprising the first sensor and the second sensor; or
the at least one external device comprises a first external device comprising the first sensor, and a second external device comprising the second sensor.

72. The system of any one of claims 64 to 71, wherein the processor is further configured to identify a user physiological parameter that the user physiological data relates to.

73. The system of claim 72, wherein the processor is further configured to send a prompt to confirm that the at least one operation parameter should be adjusted based on the identified user physiological parameter to a display.

74. The system of any one of claims 64 to 73, wherein the communications interface is configured to establish communication manually or automatically.

75. The system of any one of claims 64 to 74, wherein the at least one external device comprises a plurality of external devices.

76. The system of any one of claims 64 to 75, wherein the at least one operational parameter relates to at least one of:
a dosage amount;
a dosage frequency; and
a dosage time.

77. The system of any one of claims 64 to 76, wherein the user physiological data relates to at least one of:
blood concentration of dispensed drug;
blood glucose level; and
heart rate.

78. The system of any one of claims 64 to 77, wherein the at least one external device is an implanted device.

79. The system of any one of claims 64 to 78, wherein the drug holder is configured to hold a drug, the dispensing mechanism is configured to dispense the drug, and the drug comprises at least one of infliximab, golimumab, ustekinumab, daratumumab, guselkumab, epoetin alfa, risperidone, esketamine, ketamine, and paliperidone palmitate.

80. A drug administration device, comprising:
a housing that accommodates a drug holder and a dispensing mechanism;
a communications interface configured to:
scan for at least one external device with which to establish communication;
establish communication between the drug administration device and the at least one external device; and
receive user physiological data from the at least one external device; and
a processor in communication with the communications interface, the processor configured to adjust at least one operational parameter of the drug administration device based on the user physiological data.

81. The device of claim 80, wherein the user physiological data comprises a first user physiological parameter and a second user physiological parameter different from the first user physiological parameter, and wherein the processor is further configured to:
adjust the at least one operational parameter based on the first user physiological parameter; and
adjust the at least one operational parameter based on the second user physiological parameter.

82. The device of claim 81, wherein the processor is further configured to adjust the at least one operational parameter based on the first user physiological parameter if the second user physiological parameter is within a first range, and to adjust the at least one operational parameter based on the second user physiological parameter if the second user physiological parameter is within a second range.

83. The device of claim 82, wherein a limit of the first range is equal to a threshold value, and the threshold value is a boundary of the second range.

84. The device of claim 82 or claim 83, wherein when the second user physiological parameter is within the second range the processor is further configured to:
determine an acceptable range of adjustment values of the at least one operational parameter based on the first user physiological parameter;
determine a first adjustment value of the at least one operational parameter based on the second user physiological parameter; and
adjust the at least one operational parameter to the first adjustment value if the first adjustment value is within the acceptable range of adjustment values, and adjust the at least one operational parameter based on the first user physiological parameter, and optionally the second user physiological parameter, if the first adjustment value is outside the acceptable range of adjustment values.

85. The device of claim 84, wherein the processor is further configured to adjust the at least one operational parameter based on the first user physiological parameter and the second user physiological parameter when the first adjustment value is outside the acceptable range of adjustment values.

86. The device of any one of claims 81 to 83, wherein the processor is further configured to adjust the at least one operational parameter based on the first user physiological parameter and the second user physiological parameter.

87. The device of any one of claims 81 to 86, wherein a first sensor is configured to measure the first user physiological parameter and a second sensor is configured to measure the second user physiological parameter, and wherein either:
the at least one external device comprises a first external device comprising the first sensor and the second sensor; or
the at least one external device comprises a first external device comprising the first sensor, and a second external device comprising the second sensor.

88. The device of any one of claims 80 to 87, wherein the processor is further configured to identify a user physiological parameter that the user physiological data relates to.

89. The device of claim 88, wherein the processor is further configured to send a prompt to confirm that the at least one operation parameter should be adjusted based on the identified user physiological parameter to a display.

90. The device of any one of claims 80 to 89, wherein the communications interface is configured to establish communication manually or automatically.

91. The device of any one of claims 80 to 90, wherein the at least one external device comprises a plurality of external devices.

92. The device of any one of claims 80 to 91, wherein the at least one operational parameter relates to at least one of:
a dosage amount;
a dosage frequency; and
a dosage time.

93. The device of any one of claims 80 to 92, wherein the user physiological data relates to at least one of:
blood concentration of dispensed drug;
blood glucose level; and
heart rate.

94. The device of any one of claims 80 to 93, wherein the drug holder is configured to hold a drug, the dispensing mechanism is configured to dispense the drug, and the drug comprises at least one of infliximab, golimumab, ustekinumab, daratumumab, guselkumab, epoetin alfa, risperidone, esketamine, ketamine, and paliperidone palmitate.

95. A method of assessing whether to update a control program of a drug administration device, the method comprising:
establishing a communication link between the drug administration device and a network or an external device;
sending an update request from the network or the external device to the drug administration device;
confirming, on the drug administration device, whether the update request is authorized; and
if the update request is authorized within a predetermined time frame, updating one or more parameters of the control program of the drug administration device, or
if the update request is not authorized within the predetermined time frame preventing the drug administration device from updating any parameters of the control program.

96. The method of claim 95, wherein confirming whether an update request is authorized comprises confirming whether an input from a user has occurred on the drug administration device.

97. The method of claim 96, further comprising prompting the user to provide an input prior to confirming whether the input from the user has occurred on the drug administration device.

98. The method of any one of claims 95 to 97, wherein confirming whether an update request is authorized comprises confirming whether the external device or network is an acceptable external device or network.

99. The method of any one of claims 95 to 98, wherein establishing the communication link comprises:
communicating a digital signature from the network or the external device to the drug administration device; and
confirming an identity of the digital signature is valid on the drug administration device.

100. The method of any one of claims 95 to 99, further comprising storing a digital characteristic of the communication link or the update request if the update request is not authorized within the predetermined time frame.

101. The method of any one of claims 99 to 100, further comprising comparing the stored digital characteristic against a pre-defined list of known digital characteristics stored in the drug administration device.

102. The method of any one of claims 95 to 101, further comprising implementing a first security protocol if the update request is not authorized within the predetermined time frame.

103. The method of claim 102, when dependent on claim 101, wherein the first security protocol comprises implementing a counter-measure if the stored digital characteristic of the communication link or the update request matches a known digital characteristic in the pre-defined list.

104. The method of any one of claims 102 to 103, wherein the first security protocol comprises notifying an external system.

105. The method of any one of claims 102 to 104, wherein the first security protocol comprises, after a predetermined number of failed update authorizations, locking the drug administration device into a locked state, wherein the drug administration device in the locked state is prevented from communicating remotely.

106. The method of claim 105, further comprising requiring an input from a user to be received on the drug administration device to resume remote communication when the drug administration device is in the locked state.

107. The method of any one of claims 95 to 106, further comprising requiring a second update authorization to be confirmed on either the external device or a control device connected to the network within a second predetermined time frame.

108. The method of any one of claims 95 to 107, wherein the one or more parameters of the control program of the drug administration device are stored in an electronic memory.

109. The method of any one of claims 95 to 108, wherein the one or more parameters of the control program comprise one of the following: drug dosage, frequency of drug administration, and duration of drug administration.

110. The method of any one of claims 95 to 109, wherein the external device is an internet-connected device.

111. The method of any one of claims 95 to 110, wherein the drug administration device is configured to deliver a drug, and the drug comprises at least one of infliximab, golimumab, ustekinumab, daratumumab, guselkumab, epoetin alfa, risperidone, esketamine, ketamine, and paliperidone palmitate.

112. A drug administration device comprising a control program for administering a drug, wherein the drug administration device is configured to update the control program in response to an update request sent from a network or an external device;
wherein the drug administration device comprises a processor and a memory in communication with the processor;
wherein the drug administration device is configured to require a local authorization to authorize the update request; and
wherein the processor is configured to update the control program based on data from the network or the external device if the update request is authorized on the drug administration device, and wherein the processor is configured to prevent the control program from updating if the update request is not authorized on the drug administration device.

113. The drug administration device of claim 112, wherein the drug administration device comprises a user interface device.

114. The drug administration device of claim 113, wherein the local authorization is received via the user interface device.

115. The drug administration device of any one of claims 112 to 114, wherein the drug administration device is configured to confirm the identity of a digital signature received from the network or the external device.

116. The drug administration device of any one of claims 112 to 115, wherein the processor of the drug administration device is configured to store a digital characteristic of the communication link or the update request in the memory of the drug administration device if the update request is not authorized within the predetermined time frame.

117. The drug administration device of any one of claims 112 to 116, wherein the processor is configured to place the drug administration device into a locked state in response to a security protocol, wherein the drug administration device is prevented from any wireless communication in the locked state.

118. The drug administration device of any one of claims 112 to 117, wherein, in response to a security protocol, the drug administration device is configured to communicate an error message to an indicator or to the user interface device, to notify a user.

119. The device of any one of claims 112 to 118, wherein the drug administration device is configured to deliver a drug, and the drug comprises at least one of infliximab, golimumab, ustekinumab, daratumumab, guselkumab, epoetin alfa, risperidone, esketamine, ketamine, and paliperidone palmitate.

120. A drug administration system, comprising
a first drug administration device configured to administer a first drug and configured to operate in accordance with a first control program; and
a second drug administration device configured to administer a second drug;
wherein the first drug administration device is configured to communicate with the second drug administration device; and
wherein the first drug administration device comprises
a processor; and
a memory configured to communicate with the processor, wherein the memory is configured to store at least one dosing parameter of the first control program; and
wherein the processor is configured to adjust the at least one dosing parameter in response to a communication received from the second drug administration device.

121. A drug administration system, comprising
a first drug administration device configured to administer a first drug and configured to operate in accordance with a first control program;
a second drug administration device configured to administer a second drug; and
an external device;
wherein the first drug administration device is configured to communicate with the second drug administration device; and
wherein the external device comprises
a processor; and
a memory configured to communicate with the processor, wherein the memory is configured to store at least one dosing parameter of the first control program; and
wherein the processor is configured to adjust the at least one dosing parameter in response to a communication received from the second drug administration device.

122. The drug administration system of any one of claims 120 to 121, wherein the second drug administration device is configured to operate in accordance with a second control program.

123. The drug administration system of claim 121, or claim 122, when dependent on claim 121, wherein the external device is configured to communicate with the first drug administration device and the second drug administration device.

124. The drug administration system of any one of claims 122 to 123, wherein one of the first drug administration device or the external device is a primary controller, wherein the primary controller is configured to operate the first drug administration device in accordance with the first control program and operate the second drug administration device in accordance with the second control program.

125. The drug administration system of claim 120, or any one of claims 122 to 124, when dependent on claim 120, wherein the first drug administration device is configured to receive the communication from the second drug administration device directly from the second drug administration device.

126. The drug administration system of any one of claim 121, or any one of claims 122 to 124, when dependent on claim 121, the first drug administration device is configured to receive the communication from the second drug administration device indirectly from the second drug administration device via the external device.

127. The drug administration system of any one of claims 120 to 126, further comprising a set of interaction parameters stored in the memory, the interaction parameters comprising dosing requirements based on the combination of the first drug and the second drug.

128. The drug administration system of any one of claims 122 to 127, wherein the processor is configured to delay operation of the first drug administration device in accordance with the first control program based on the operation of the second drug administration device in accordance with the second control program.

129. The drug administration system of any one of claims 122 to 127, wherein the processor is configured to avoid simultaneous operation of the first drug administration device in accordance with the first control program and operation of the second drug administration device in accordance with the second control program.

130. The drug administration system of claim 129, wherein the processor is configured to:
measure an elapsed time after an event associated with a control program,
compare the elapsed time against an interaction parameter, and
delay operation of the first drug administration device in accordance with the first control program if sufficient time has not passed.

131. The drug administration system of any one of claims 120 to 130, wherein the first drug administration device and the second drug administration device share a housing.

132. The drug administration system of any one of claims 120 to 131, wherein the first drug administration device and the second drug administration device share a component for drug delivery or communication.

133. The drug administration system of any one of claims 120 to 132, wherein the first drug administration device and the second drug administration device are configured to communicate wirelessly.

134. The drug administration system of claim any one of claims 120 to 133, further comprising a physiological sensor configured to sense a body parameter.

135. The drug administration system of claim 134, wherein the physiological sensor is configured to communicate the sensed body parameter to the first drug administration device, the second drug administration device, or the external device.

136. The drug administration system of claim 135, wherein the at least one dosing parameter of the first control program is adjusted in response to the sensed body parameter.

137. The drug administration system of claim 135, wherein the processor is configured to delay operation of the first drug administration device in accordance with the first control program in response to the sensed body parameter.

138. The drug administration system of any one of claims 120 to 137, wherein the at least one dosing parameter of the first control program is configured to be updated in response to a manual input.

139. The drug administration system of any one of claims 123 to 138, wherein the first drug administration device is configured to notify a user when the first drug administration device fails to receive a communication from the second drug administration device or the external device.

140. The drug administration system of any one of claims 120 to 139, wherein the dosing parameters of the first control program are configured to be reset to default dosing parameters when the first drug administration device fails to receive a communication from the second drug administration device or the external device.

141. The drug administration system of any one of claims 120 to 140, wherein the at least one dosing parameter of the first control program comprises any of the following: duration of drug administration, dosage of medication, frequency of drug administration, and time of administration.

142. The drug administration system of any preceding claim, wherein the first drug and the second drug each comprise at least one of infliximab, golimumab, ustekinumab, daratumumab, guselkumab, epoetin alfa, risperidone, esketamine, ketamine, and paliperidone palmitate.

143. A drug administration device configured to operate in accordance with a control program, the drug administration device comprising a processor and a memory configured to communicate with the processor;
wherein the memory is configured to store at least one dosing parameter of the control program; and
wherein the drug administration device is configured to communicate with a second drug administration device, and the drug administration device is configured to adjust at least one dosing parameter of the control program in response to a communication received from the second drug administration device.

144. The drug administration device of claim 143, wherein the drug administration device is configured to communicate with an external device.

145. The drug administration device of claim 143 or claim 144, wherein the drug administration device is configured to receive the communication from the second drug administration device indirectly via the external device.

146. The drug administration device of any one of claims 143 to 145, wherein the processor is configured to delay operation of the drug administration device in accordance with the control program.

147. The drug administration device of any one of claims 143 to 146, wherein the processor is configured to avoid simultaneous operation of the drug administration device in accordance with the control program and operation of the second drug administration device in accordance with a second control program.

148. The drug administration device of any one of claims 143 to 147, wherein the drug administration device is configured to communicate wirelessly to the second drug administration device.

149. The drug administration device of any one of claims 143 to 148, wherein the drug administration device is configured to deliver a drug, and the drug comprises at least one of infliximab, golimumab, ustekinumab, daratumumab, guselkumab, epoetin alfa, risperidone, esketamine, ketamine, and paliperidone palmitate.

150. A method of administering a drug with a drug administration device, comprising:
receiving a communication on the drug administration device from a second drug administration device;
determining whether to adjust a dosing parameter of a first control program on the drug administration device as a result of the communication;
adjusting the dosing parameter of the first control program if it is determined to adjust the dosing parameter; and
administering the drug by the drug administration device in accordance with the first control program.

151. The method of claim 150, wherein the drug administration device receives the communication from the second drug administration device via an external device.

152. The method of any one of claim 150 or 151, wherein the determination of whether to adjust the dosing parameter is based on an interaction parameter, stored in the dug administration device, and an elapsed time.

153. The method of any one of claims 150 to 152, wherein the drug administration device receives the communication from the second drug administration device wirelessly.

154. The method of any one of claims 150 to 153, further comprising adjusting one or more dosing parameters of the first control program, wherein the one or more dosing parameters are adjusted based on a sensed body parameter sensed by a physiological sensor.

155. The method of claim 154, wherein the dosing parameters are adjusted when the sensed body parameter exceeds a predetermined threshold.

156. The method of any one of claims 150 to 155, wherein the drug administration device is configured to deliver a drug, and the drug comprises at least one of infliximab, golimumab, ustekinumab, daratumumab, guselkumab, epoetin alfa, risperidone, esketamine, ketamine, and paliperidone palmitate.
